(19) United States
(12) Patent Application Publication
(10) Pub. No.: US 2016/0304466 A1
(43) Pub. Date: Oct. 20, 2016

(54) NOVEL COMPOUNDS AS JNK KINASE INHIBITORS

(71) Applicant: THE SCRIPPS RESEARCH INSTITUTE, La Jolla, CA (US)

(72) Inventors: Yangbo Feng, Palm Beach Gardens, FL (US); Philip Lograsso, Jupiter, FL (US); Ke Zheng, Palm Beach Gardens, FL (US); Chul Min Park, Junggu (KR)

(21) Appl. No.: 15/101,738

(22) PCT Filed: Dec. 3, 2014
(86) PCT No.: PCT/US14/68333

§ 371 Date: Jun. 3, 2016

Related U.S. Application Data
(60) Provisional application No. 61/911,741, filed on Dec. 4, 2013.
(60) Provisional application No. 62/001,872, filed on May 22, 2014.

(51) Int. Cl. C07D 231/40; C07D 401/14; C07D 409/14; C07D 403/12; C07D 239/47; C07D 405/12; C07D 417/12; C07D 417/14; C07D 471/04; C07D 487/04; C07D 401/12; C07D 403/14

(52) U.S. Cl. CPC C07D 231/40; C07D 401/12; C07D 401/14; C07D 409/14; C07D 403/12; C07D 403/14; C07D 405/12; C07D 417/12; C07D 417/14; C07D 471/04; C07D 487/04; C07D 239/47

(57) ABSTRACT

The present invention is directed to modulators, such as inhibitors, of JNK isoform 2 (JNK2) or isoform 3 (JNK3) comprising compounds of formula (I) or formula (II) as described herein. Compounds of the invention can be used for treatment of a medical disorder in a patient wherein modulation of JNK3 is medically indicated, such as when the disorder is Parkinson disease (PD) Alzheimer's disease (AD), Huntington's disease (HD), amyotrophic lateral sclerosis (ALS), multiple sclerosis (MS), myocardial infarction (MI), glaucoma, obesity, diabetes, cancer, rheumatoid arthritis, fibrotic disease, pulmonary fibrosis, kidney disease, liver inflammation, Crohns disease, hearing loss, Prader Willi syndrome, or a condition where modification of feeding behavior is medically indicated.

### CROSS-REFERENCE TO RELATED APPLICATIONS

This application claims the priority of U.S. Ser. No. 61/911,741, filed Dec. 4, 2013, and 62/001,872, filed May 22, 2014, the disclosures of which are incorporated herein by reference in their entireties.

### STATEMENT OF GOVERNMENT SUPPORT

This invention was made with government support under W81XWH-12-1-0431 and AL11003 awarded by the Department of Defense. The U.S. government has certain rights in the invention.

### BACKGROUND

The mitogen activated protein (MAP) kinase family member c-jun-N-terminal kinase (JNK) has been shown to be a compelling therapeutic target for a variety of diseases including neurodegeneration, metabolic disorders, inflammation, cardiovascular disease, and cancer. Validation for JNK as a therapeutic target has come from studies employing knock out (KO) mice, peptide inhibitors of JNK and small molecule ATP competitive inhibitors of JNK. JNK is known to exist in various isoforms, such as isoform 1 (JNK1), isoform 2 (JNK2), and isoform 3 (JNK3).

The design and identification of potent and highly selective JNK inhibitors, has been pursued in the past few years due to potential wide spread therapeutic applications. In particular, development of brain penetrant small molecule inhibitors for JNK has been a major focus in order to develop efficacious therapeutics for Parkinson's disease (PD) and other neurodegenerative diseases, such as Alzheimer's disease (AD), Huntington's disease (HD), amyotrophic lateral sclerosis (ALS) and multiple sclerosis (MS). Additionally, inhibition of JNK is believed to be an effective approach for development of therapeutic compounds for treatment of myocardial infarction (MI), obesity, diabetes, glaucoma, cancer, rheumatoid arthritis, fibrotic disease, pulmonary fibrosis, kidney disease, liver inflammation, Crohn's disease, and hearing loss (see, for example: Eshraghi A A, et al., Blocking c-Jun-N-terminal kinase signaling can prevent hearing loss induced by both electrode insertion trauma and neomycin ototoxicity, Hear Res. 2007 April; 226(1-2):168-77; J. Wang, et al., A Peptide Inhibitor of c-Jun N-Terminal Kinase Protects against Both Aminoglycoside and Acoustic Trauma-Induced Auditory Hair Cell Death and Hearing Loss, The Journal of Neuroscience, Sep. 17, 2003, 23(24):8596-8607).

### SUMMARY

The invention is directed in various embodiments to potent modulators of JNK, in particular modulators of JNK isoform 2 (JNK2) or of JNK isoform 3 (JNK3), or of both, relative to, e.g., JNK1; and to methods of treatment of medical conditions wherein selective inhibition of JNK, e.g., of JNK isoform 2 or 3, is medically indicated. Medical conditions that can be treated including for treatment of myocardial infarction (MI), obesity, diabetes, Parkinson's disease, Alzheimer's disease, ALS, glaucoma, cancer, rheumatoid arthritis, fibrotic disease, pulmonary fibrosis, kidney disease, liver inflammation, Crohn's disease, hearing loss, or Prader Willi syndrome, or a condition where modification of feeding behavior is medically indicated.

In various embodiments, the invention provides a JNK isoform 2 or 3 modulator of formula (I)

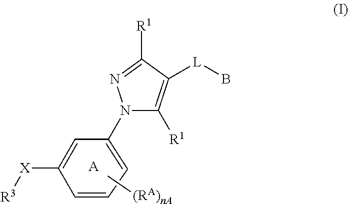

wherein

R¹ is independently at each occurrence H, CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkoxy, alkyl or cycloalkyl of R¹ is substituted with 0-3 (C₁-C₆)alkyl, (C₁-C₆)alkoxy, CN, CF₃, or halo;

ring A comprises 0-2 nitrogen atoms therein, provided that R³—X, the pyrazole bearing R¹, and any R^{A}, is bonded to a carbon atom of ring A; wherein R^{A} is independently at each occurrence CN, CF₃, halo, (C₁-C₆)alkyl, (C₁-C₆)alkoxy, (C₃-C₉)cycloalkyl, or (C₃-C₉)cycloalkoxy, wherein there are 0, 1, or 2 replacements of a respective methylene carbon atom of the alkyl, alkoxy, cycloalkyl, or cycloalkoxy by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkyl, alkoxy, cycloalkyl, or cycloalkoxy of R^{A} is substituted with 0-3 (C₁-C₆)alkoxy, CN, CF₃, or halo; and nA is 0, 1, 2, or 3, provided that nA is not greater than the number of carbon atoms in ring A minus two;

linker L is a bond, (CR′₂)ₙO(CR′₂)ₙ, (CR′₂)ₙ(N(R²))ₘ(CR′₂)ₙ, (CR′₂)ₙC(═O)(N(R²))ₘ(CR′₂)ₙ, (CR′₂)ₙ(N(R²))ₘC(═O)(N(R²))ₘ((CR′₂)ₙ, (CR′₂)ₙ(N(R′))ₘC(═S)(N(R′))ₘ(CR′₂)ₙ, (CR′₂)ₙOC(═O)(N(R²))ₘ(CR′₂)ₙ, (CR′₂)ₙSO₂(N(R²))ₘ(CR′₂)ₙ, or (CR′₂)ₙS(O)_{q}(CR′₂)ₙ wherein m is independently at each occurrence 1 or 2, n independently at each occurrence is 0, 1, 2, or 3, and q=0, 1, or 2;

R′ is independently at each occurrence selected from the group consisting of H, (C₁-C₆)alkyl, and (C₁-C₆)acyl, wherein any alkyl or acyl of R′ is substituted with 0, 1, or 2 independently selected R₂N or OR groups;

R is H or (C₁-C₆)alkyl, wherein alkyl is substituted with 0-3 (C₁-C₆)alkyl, (C₁-C₆)alkoxy, hydroxyl, NH₂, mono- or dialkylamino, CN, CF₃, or halo

R² is independently at each occurrence H, (C₁-C₆)alkyl, (C₁-C₆)acyl, or (C₃-C₉)cycloalkyl wherein there are 0, 1, or 2 replacements of a respective methylene carbon atom of the alkyl, acyl, or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkyl or cycloalkyl of R² is substituted with 0-3 (C₁-C₆)alkoxy, OH, R₂N, CN, CF₃, or halo; and,

B is (C₁-C₆)alkyl, (C₂-C₆)alkenyl, (C₂-C₆)alkynyl, mono- or bicyclic (C₆-C₁₀)aryl, mono- or bicyclic (C₆-C₁₀)aryloxy, mono- or bicyclic (C₆-C₁₀)aryl(C₁-C₆)alkyl, mono- or bicyclic (C₆-C₁₀) aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of B is substituted with 0-3 R^{B};

wherein R^{B} is independently at each occurrence CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkoxy, alkyl or cycloalkyl of R^{B} is substituted with 0-3 (C₁-C₆)alkoxy, CN, CF₃, or halo; or,

B and R², together with the nitrogen atom to which they are bonded, together form a 3-10 membered mono- or bicyclic heterocyclyl or heteroaryl, substituted with 0-3 (C₁-C₆)alkoxy, CN, CF₃, or halo;

X is a bond, (CR′₂)ₙO(CR′₂)ₙ, (CR′₂)ₙ(N(R′))ₘ(CR′₂)ₙ, (CR′₂)ₙC(═O)(N(R′))ₘ(CR′₂)ₙ, (CR′₂)ₙ(N(R′))ₘC(═O)(N(R′))m(CR′₂)ₙ, (CR′₂)ₙ(N(R′))ₘC(═S)(N(R′))ₘ(CR′₂)ₙ, (CR′₂)ₙOC(═O)(N(R′))ₘ(CR′₂)ₙ, (CR′2)nSO₂(N(R′))ₘ(CR′₂)ₙ, or (CR′₂)ₙS(O)_{q}(CR′₂)ₙ wherein m is independently at each occurrence 1 or 2, n is independently at each occurrence is 0, 1, 2, or 3, and q=0, 1, or 2;

R³ is H, (C₁-C₆)alkyl or (C₃-C₉)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′), wherein any alkyl or cycloalkyl is substituted with 0-3 (C₁-C₆)alkoxy, OH, R₂N, CN, CF₃, or halo; or R³ is (C₆-C₁₀) mono- or bicyclic aryl, or 3-10 membered mono- or bicyclic heteroaryl, wherein any aryl or heteroaryl of R³ is substituted with 0-3 R⁴; provided that if X is a bond, R³ is not H;

R⁴ is OH, R₂N, CN, CF₃, halo, (C₁-C₆)alkoxy, (C₁-C₆)alkyl or (C₃-C₉)cycloalkyl wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkoxy, alkyl or cycloalkyl of R⁴ is substituted with 0-3 (C₁-C₆)alkoxy, OH, R₂N, CN, CF₃, or halo, or R⁴ is mono- or bicyclic (C₆-C₁₀)aryl, mono- or bicyclic (C₆-C₁₀)aryloxy, mono- or bicyclic (C₆-C₁₀)aryl(C₁-C₆)alkyl, mono- or bicyclic (C₆-C₁₀) aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of R⁴ is substituted with 0-3 (C₁-C₆)alkoxy, (C₁-C₆)alkyl, CN, CF₃, or halo;

or a salt thereof.

Numerous specific examples are provided, including compounds of formula (IA), as described below.

In various embodiments, the invention provides a compound of formula

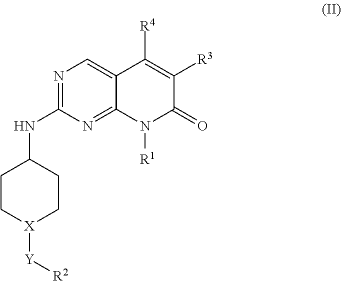

wherein

X is N or CH; when X is N, Y is absent; when X is CH, Y is NR′ or is O;

R¹ is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, 0, C(═S), C(═O), C(═O)O, C(═O)C(═O), C(═O)NR′, NR′C(═O), NR′C(═O)O, OC(═O)NR′, SO₂NR′, NR′SO₂, NR′SO₂NR′, C(═O)NR′NR′, or NR′C(═O)NR′;

R² is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, 0, C(═S), C(═O), C(═O)O, C(═O)C(═O), C(═O)NR′, NR′C(═O), NR′C(═O)O, OC(═O)NR′, SO₂NR′, NR′SO₂, NR′SO₂NR′, C(═O)NR′NR′, or NR′C(═O)NR′; or R² is (C₆-C₁₀) aryl, (C₆-C₁₀)aryl(C₁-C₆)alkyl, a 5-10 membered heteroaryl, or a 5-10 membered heteroaryl-(C₁-C₆)alkyl, wherein any aryl or heteroaryl is unsubstituted or is substituted with 1, 2, or 3 J groups; or R² is C(═O)OR, C(═O)R, or C(═O)NR₂;

R and R′ are independently at each occurrence H or (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, 0, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;

R³ and R⁴ are each independently H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, 0, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;

or a pharmaceutically acceptable salt thereof.

In various embodiments, the invention provides a use or a method of treatment with a compound of formula (I) or formula (II) for a medical disorder wherein modulation of JNK, such as JNK isoform 2 or 3, is medically indicated. The disorder can be Parkinson's disease (PD) Alzheimer's (AD), Huntington's disease (HD), amyotrophic lateral sclerosis (ALS) multiple sclerosis (MS), myocardial infarction (MI), obesity, diabetes, Alzheimer's disease, ALS, Crohn's disease, hearing loss, Prader Willi syndrome, or a condition where modification of feeding behavior is medically indicated.

### DETAILED DESCRIPTION

### Overview

We have chosen to develop small molecule JNK inhibitors, e.g., JNK2 inhibitors, JNK3 inhibitors, or both, as therapeutic agents to treat disorders such as Parkinson's disease (PD) Alzheimer's (AD), Huntington's disease (HD), amyotrophic lateral sclerosis (ALS) multiple sclerosis (MS), myocardial infarction (MI), obesity, diabetes, Alzheimer's disease, ALS, cancer, rheumatoid arthritis, fibrotic disease, pulmonary fibrosis, kidney disease, liver inflammation, Crohn's disease, hearing loss, Prader Willi syndrome, or a condition where modification of feeding behavior is medically indicated. The development of inhibitors having a high degree of selectivity is expected to afford lower toxicity risk for development candidates for treatment of the conditions associated with JNK. In addition, by targeting the substrate site in JNK, and potentially blocking JNK mitochondrial translocation, we may be able to provide an inhibition mechanism that prevents mitochondrial dysfunction and cardiomyocyte cell death. Indeed, mitochondrial function specific assays enable us to monitor several measures of mitochondrial function that contribute to cell death. For example, mitochondrial functional assays measuring ROS and mitochondrial membrane potential (MMP) have not been reported for cardiomyocytes. The robust, high-throughput nature of all these assays can support detailed medicinal chemistry efforts for discovery of novel structural classes and mechanisms of inhibition for JNK. Finally, small molecule inhibitors that do not behave as covalent modifiers and non-covalently bind in the ATP and substrate pockets of JNK have not been reported, and novel structures associated with this approach have been developed by the inventors herein.

### DEFINITIONS

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein JNK plays a role in the biochemical mechanisms involved in the disease or malcondition or symptom(s) thereof such that a therapeutically beneficial effect can be achieved by acting on a kinase, specifically by acting to inhibit the bioactivity of an isoform of JNK such as JNK1, 2, or 3. “Acting on” JNK, or “modulating” JNK, can include binding to JNK and/or inhibiting the bioactivity of JNK and/or allosterically regulating the bioactivity of JNK in vivo. “Selectively” modulating or inhibiting JNK3, i.e., JNK isoform 3, refers to modulation or inhibition of JNK3 relative to another JNK isoform such as JNK1. Likewise, “selectively” modulating or inhibiting JNK2, i.e., KNK isoform 2, refers to modulation or inhibition of JNK2 relative to another JNK isoform such as JNK1. A selective JNK isoform 2 or isoform 3 modulator can modulate either or both of these isoforms relative to JNK1.

The expression “effective amount”, when used to describe therapy to an individual suffering from a disorder, refers to the amount of a compound of the invention that is effective to inhibit or otherwise act on JNK2 or JNK3 in the individual's tissues wherein JNK2 or JNK3, respectively, involved in the disorder is active, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

All chiral, diastereomeric, racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

C(O) and S(O)₂ groups can also be bound to one or two heteroatoms, such as nitrogen or oxygen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a “urea.” When a C(O) is bonded to one oxygen and one nitrogen atom, the resulting group is termed a “carbamate” or “urethane.” When a S(O)₂ group is bound to one carbon and one nitrogen atom, the resulting unit is termed a “sulfonamide.” When a S(O)₂ group is bound to two nitrogen atoms, the resulting unit is termed a “sulfamide.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a hetero atom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art. As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups.

For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, thiadiazolyl, pyridinyl, pyrimidinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The term “cycloalkoxy” as used herein refers to a cycloalkyl group attached to oxygen (cycloalkyl-O—). Examples of cyclic alkoxy include but are not limited to cyclopropyloxy, cyclobutyloxy, cyclopentyloxy, cyclohexyloxy, and the like. Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, and the like.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “halo acyl” group. An example is a trifluoroacetyl group.

The term “amine” includes primary, secondary, and tertiary amines having, e.g., the formula N(group)₃ wherein each group can independently be H or non-H, such as alkyl, aryl, and the like Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” group is a group of the formula C(O)NR₂, wherein R can be H, alkyl, aryl, etc.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention. “Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

### Isomerism and Tautomerism in Compounds of the Invention

### Tautomerism

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

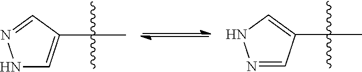 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

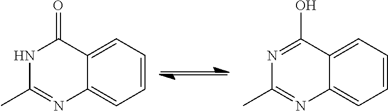

is an example of tautomerism.

Accordingly, a structure depicted herein as one tautomer is intended to also include the other tautomer.

### Optical Isomerism

It will be understood that when compounds of the present invention contain one or more chiral centers, the compounds may exist in, and may be isolated as single and substantially pure enantiomeric or diastereomeric forms or as racemic mixtures. The present invention therefore includes any possible enantiomers, diastereomers, racemates or mixtures thereof of the compounds of the invention.

The compounds of the invention, or compounds used in practicing methods of the invention, may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present invention encompasses various stereoisomers of these compounds and mixtures thereof.

Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the invention, or compounds used in practicing methods of the invention, may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of contemplated compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking Once the priority ranking of the four groups is determined, the molecule is oriented so that the lowest ranking group is pointed away from the viewer. Then, if the descending rank order of the other groups proceeds clockwise, the molecule is designated as having an (R) absolute configuration, and if the descending rank of the other groups proceeds counterclockwise, the molecule is designated as having an (S) absolute configuration. In the example in the Scheme below, the Cahn-Ingold-Prelog ranking is A>B>C>D. The lowest ranking atom, D is oriented away from the viewer. The solid wedge indicates that the atom bonded thereby projects toward the viewer out of the plane of the paper, and a dashed wedge indicates that the atom bonded thereby projects away from the viewer out of the plan of the paper, i.e., the plane “of the paper” being defined by atoms A, C, and the chiral carbon atom for the (R) configuration shown below.

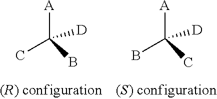

A carbon atom bearing the A-D atoms as shown above is known as a “chiral” carbon atom, and the position of such a carbon atom in a molecule is termed a “chiral center.” Compounds of the invention may contain more than one chiral center, and the configuration at each chiral center is described in the same fashion.

There are various conventions for depicting chiral structures using solid and dashed wedges. For example, for the (R) configuration shown above, the following two depictions are equivalent:

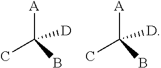

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” or “isolated enantiomer” means a compound which has been substantially purified from the corresponding optical isomer(s) of the same formula. Preferably, the isolated isomer is at least about 80%, more preferably at least 90% enantiomerically pure, even more preferably at least 98% enantiomerically pure, most preferably at least about 99% enantiomerically pure, by weight. By “enantiomeric purity” is meant the percent of the predominant enantiomer in an enantiomeric mixture of optical isomers of a compound. A pure single enantiomer has an enantiomeric purity of 100%.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

Another well-known method of obtaining separate and substantially pure optical isomers is classic resolution, whereby a chiral racemic compound containing an ionized functional group, such as a protonated amine or carboxylate group, forms diastereomeric salts with an oppositely ionized chiral nonracemic additive. The resultant diastereomeric salt forms can then be separated by standard physical means, such as differential solubility, and then the chiral nonracemic additive may be either removed or exchanged with an alternate counter ion by standard chemical means, or alternatively the diastereomeric salt form may retained as a salt to be used as a therapeutic agent or as a precursor to a therapeutic agent.

Accordingly, in various embodiments, the invention can provide a compound of formula (I)

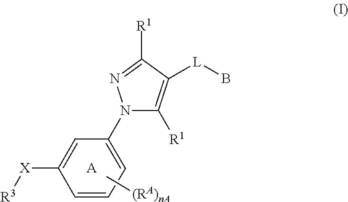

wherein

R¹ is independently at each occurrence H, CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkoxy, alkyl or cycloalkyl of R¹ is substituted with 0-3 (C₁-C₆)alkyl, (C₁-C₆)alkoxy, CN, CF₃, or halo;

ring A comprises 0-2 nitrogen atoms therein, provided that R³—X, the pyrazole bearing R¹, and any R^{A}, is bonded to a carbon atom of ring A; wherein R^{A} is independently at each occurrence CN, CF₃, halo, (C₁-C₆)alkyl, (C₁-C₆)alkoxy, (C₃-C₉)cycloalkyl, or (C₃-C₉)cycloalkoxy, wherein there are 0, 1, or 2 replacements of a respective methylene carbon atom of the alkyl, alkoxy, cycloalkyl, or cycloalkoxy by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkyl, alkoxy, cycloalkyl, or cycloalkoxy of R^{A} is substituted with 0-3 (C₁-C₆)alkoxy, CN, CF₃, or halo; and nA is 0, 1, 2, or 3, provided that nA is not greater than the number of carbon atoms in ring A minus two;

linker L is a bond, (CR′₂)ₙO(CR′₂)ₙ, (CR′₂)ₙ(N(R²))ₘ(CR′₂)ₙ, (CR′₂)ₙC(═O)(N(R²))ₘ(CR′₂)ₙ, (CR′₂)ₙ(N(R²))ₘC(═O)(N(R²))ₘ(CR′₂)ₙ, (CR′2)ₙ(N(R′))ₘC(═S)(N(R′))ₘ(CR′₂)ₙ, (CR′₂)ₙOC(═O)(N(R²))ₘ(CR′₂)ₙ, (CR′₂)ₙSO₂(N(R²))ₘ(CR′₂)ₙ, or (CR′₂)ₙS(O)_{q}(CR′₂)ₙ wherein m is independently at each occurrence 1 or 2, n independently at each occurrence is 0, 1, 2, or 3, and q=0, 1, or 2;

R′ is independently at each occurrence selected from the group consisting of H, (C₁-C₆)alkyl, and (C₁-C₆)acyl, wherein any alkyl or acyl of R′ is substituted with 0, 1, or 2 independently selected R₂N or OR groups;

R is H or (C₁-C₆)alkyl, wherein alkyl is substituted with 0-3 (C₁-C₆)alkyl, (C₁-C₆)alkoxy, hydroxyl, NH₂, mono- or dialkylamino, CN, CF₃, or halo

R² is independently at each occurrence H, (C₁-C₆)alkyl, (C₁-C₆)acyl, or (C₃-C₉)cycloalkyl wherein there are 0, 1, or 2 replacements of a respective methylene carbon atom of the alkyl, acyl, or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkyl or cycloalkyl of R² is substituted with 0-3 (C₁-C₆)alkoxy, OH, R₂N, CN, CF₃, or halo; and,

B is (C₁-C₆)alkyl, (C₂-C₆)alkenyl, (C₂-C₆)alkynyl, mono- or bicyclic (C₆-C₁₀)aryl, mono- or bicyclic (C₆-C₁₀)aryloxy, mono- or bicyclic (C₆-C₁₀)aryl(C₁-C₆)alkyl, mono- or bicyclic (C₆-C₁₀) aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of B is substituted with 0-3 R^{B};

wherein R^{B} is independently at each occurrence CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkoxy, alkyl or cycloalkyl of R^{B} is substituted with 0-3 (C₁-C₆)alkoxy, CN, CF₃, or halo; or,

B and R², together with the nitrogen atom to which they are bonded, together form a 3-10 membered mono- or bicyclic heterocyclyl or heteroaryl, substituted with 0-3 (C₁-C₆)alkoxy, CN, CF₃, or halo;

X is a bond, (CR′₂)ₙO(CR′₂)ₙ, (CR′₂)ₙ(N(R′))ₘ(CR′₂)ₙ, (CR′₂)ₙC(═O)(N(R′))ₘ(CR′₂)ₙ, (CR′₂)ₙ(N(R′))ₘC(═O)(N(R′))m(CR′₂)ₙ, (CR′₂)ₙ(N(R′))ₘC(═S)(N(R′))ₘ(CR′₂)ₙ, (CR′₂)ₙOC(═O)(N(R′))ₘ(CR′₂)ₙ, (CR′2)nSO₂(N(R′))ₘ(CR′₂)ₙ, or (CR′₂)ₙS(O)_{q}(CR′₂)ₙ wherein m is independently at each occurrence 1 or 2, n is independently at each occurrence is 0, 1, 2, or 3, and q=0, 1, or 2;

R³ is H, (C₁-C₆)alkyl or (C₃-C₉)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′), wherein any alkyl or cycloalkyl is substituted with 0-3 (C₁-C₆)alkoxy, OH, R₂N, CN, CF₃, or halo; or R³ is (C₆-C₁₀) mono- or bicyclic aryl, or 3-10 membered mono- or bicyclic heteroaryl, wherein any aryl or heteroaryl of R³ is substituted with 0-3 R⁴; provided that if X is a bond, R³ is not H;

R⁴ is OH, R₂N, CN, CF₃, halo, (C₁-C₆)alkoxy, (C₁-C₆)alkyl or (C₃-C₉)cycloalkyl wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkoxy, alkyl or cycloalkyl of R⁴ is substituted with 0-3 (C₁-C₆)alkoxy, OH, R₂N, CN, CF₃, or halo, or R⁴ is mono- or bicyclic (C₆-C₁₀) aryl, mono- or bicyclic (C₆-C₁₀)aryloxy, mono- or bicyclic (C₆-C₁₀)aryl(C₁-C₆)alkyl, mono- or bicyclic (C₆-C₁₀)aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of R⁴ is substituted with 0-3 (C₁-C₆)alkoxy, (C₁-C₆)alkyl, CN, CF₃, or halo;

or a salt thereof.

In various embodiments, a compound of the invention of formula (I) can be of formula (IA)

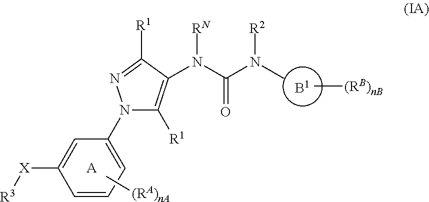

wherein ring A, R, R′, R¹, R³, R⁴, R^{A}, nA, X, and R³ are as defined in for formula (I), and wherein:

R² is H, (C1-C6)alkyl or (C3-C9)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkyl or cycloalkyl of R² is substituted with 0-3 (C1-C6)alkoxy, OH, R₂N, CN, CF₃, or halo;

R^{N} is H, or is (C1-C6)alkyl or (C3-C9)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′); wherein any alkyl or cycloalkyl of R² is substituted with 0-3 (C1-C6)alkoxy, OH, R₂N, CN, CF₃, or halo;

B¹ is mono- or bicyclic (C6-C10)aryl, mono- or bicyclic (C6-C10)aryloxy, mono- or bicyclic (C6-C10)aryl(C1-C6)alkyl, mono- or bicyclic (C6-C10)aryl(C1-C6)alkoxy, mono- or bicyclic 3-10 membered heteroaryl, mono- or bicyclic 3-10 membered heteroaryloxy, mono- or bicyclic 3-10 membered heteroaryl(C1-C6)alkyl, mono- or bicyclic 3-10 membered heteroaryl(C1-C6)alkoxy, mono- or bicyclic 3-10 membered heterocyclyl, mono- or bicyclic 3-10 membered heterocycloxy, mono- or bicyclic 3-10 membered heterocyclyl(C1-C6)alkyl, or mono- or bicyclic 3-10 membered heterocyclyl(C1-C6)alkoxy, wherein any aryl, aryloxy, heteroaryl, heteroaryloxy, heterocyclyl, or heterocycloxy, is substituted with nB R^{B} groups; nB is 0, 1, 2, or 3, and R^{B} is independently at each occurrence as defined in claim 1; or,

B¹ and R², together with the nitrogen atom to which they are bonded, together form a 3-10 membered mono- or bicyclic heterocyclyl or heteroaryl, substituted with 0-3 (C1-C6)alkoxy, CN, CF₃, or halo;

or a salt thereof.

More specifically, group X of formula (I) or of formula (IA) can be C(═O)NR′, wherein R′ is as defined herein.

More specifically, group B of formula (I) can be substituted phenyl, or group B¹ of formula (IA) can be substituted phenyl.

In various embodiments, group R³ can be substituted or unsubstituted heteroaryl or heterocyclyl.

In various embodiments, group R⁴ can be heterocyclyl or heterocyclylalkyl.

In various embodiments, a compound of the invention can be a compound of formula (IA) wherein B¹ and R², together with the nitrogen atom to which they are bonded, together form a 3-10 membered mono- or bicyclic heterocyclyl or heteroaryl, substituted with 0-3 (C1-C6)alkoxy, CN, CF₃, or halo.

More specifically, ring A can comprise 0 nitrogen atoms.

More specifically, a compound of the invention can be any one of the Examples shown below for compounds of formula (I).

In various embodiments, the invention provides a compound of formula (II)

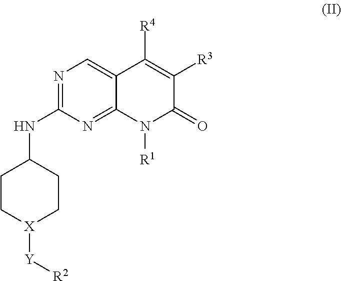

wherein

X is N or CH; when X is N, Y is absent; when X is CH, Y is NR′ or is O;

R¹ is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, 0, C(═S), C(═O), C(═O)O, C(═O)C(═O), C(═O)NR′, NR′C(═O), NR′C(═O)O, OC(═O)NR′, SO₂NR′, NR′ SO₂, NR′SO₂NR′, C(═O)NR′NR′, or NR′C(═O)NR′;

R² is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, O, C(═S), C(═O), C(═O)O, C(═O)C(═O), C(═O)NR′, NR′C(═O), NR′C(═O)O, OC(═O)NR′, SO₂NR′, NR′SO₂, NR′SO₂NR′, C(═O)NR′NR′, or NR′C(═O)NR′; or R² is (C₆-C₁₀) aryl, (C₆-C₁₀)aryl(C₁-C₆)alkyl, a 5-10 membered heteroaryl, or a 5-10 membered heteroaryl-(C₁-C₆)alkyl, wherein any aryl or heteroaryl is unsubstituted or is substituted with 1, 2, or 3 J groups; or R² is C(═O)OR, C(═O)R, or C(═O)NR₂;

R and R′ are independently at each occurrence H or (C1-C8)alkyl, (C3-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, O, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;

R³ and R⁴ are each independently H, CF₃, (C1-C8)alkyl, (C3-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} wherein q is 0, 1, or 2, O, C(═S), C(═O), C(═O)O, C(═O)C(═O), C(═O)NR′, NR′C(═O), NR′C(═O)O, OC(═O)NR′, SO₂NR′, NR′SO₂, NR′SO₂NR′, C(═O)NR′NR′, or NR′C(═O)NR′;

or a pharmaceutically acceptable salt thereof.

For example, for a compound of formula (II), X can be N and Y can be absent. Or, X can be CH and Y can be NR′. Alternatively, X can be CH and Y can be O.

For example, for a compound of formula (II), R³ and R⁴ can each be H.

For example, the compounds can be any of the Examples shown below for formula (II).

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The compounds described herein can be prepared in a number of ways based on the teachings contained herein, as described below in the Examples, and using synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. All commercially available chemicals were obtained from Aldrich, Alfa Aesare, Wako, Acros, Fisher, Fluka, Maybridge or the like and were used without further purification, except where noted. Dry solvents are obtained, for example, by passing these through activated alumina columns.

The present invention further embraces isolated compounds of the invention. The expression “isolated compound” refers to a preparation of a compound of the invention, or a mixture of compounds the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

In various embodiments, the compound or set of compounds, such as are among the inventive compounds or are used in the inventive methods, can be any one of any of the combinations and/or sub-combinations of the above-listed embodiments.

### Pharmaceutical Compositions and Methods of Treatment

In various embodiments, the invention provides pharmaceutical compositions comprising a compound of the invention and a pharmaceutically acceptable excipient.

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

In various embodiments, the invention provides the use of a compound of the invention or of a pharmaceutical composition of the invention for treatment of a disorder for which inhibition of a kinase is medically indicated. For example, the kinase can be a JNK isoform such as JNK3. For example, the disorder can be Parkinson's disease (PD) Alzheimer's (AD), Huntington's disease (HD), amyotrophic lateral sclerosis (ALS) multiple sclerosis (MS), myocardial infarction (MI), obesity, diabetes, Alzheimer's disease, ALS, Crohn's disease, hearing loss, Prader Willi syndrome, or a condition where modification of feeding behavior is medically indicated.

In various embodiments, the invention provides a method of treatment of a disorder in a patient wherein inhibition of a kinase is medically indicated, comprising administration of an effective dose of a compound of the invention or of the pharmaceutical composition of the invention. For example, the kinase can be a JNK isoform such as JNK3. For example, the disorder can be Parkinson's disease (PD) Alzheimer's disease (AD), Huntington's disease (HD), amyotrophic lateral sclerosis (ALS), multiple sclerosis (MS), myocardial infarction (MI), glaucoma, obesity, diabetes, cancer, rheumatoid arthritis, fibrotic disease, pulmonary fibrosis, kidney disease, liver inflammation, Crohn's disease, hearing loss, Prader Willi syndrome, or a condition where modification of feeding behavior is medically indicated.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

Table 1, below, provides biochemical activity data for modulators of JNK1, JNK2, and JNK3 of formula (I).

TABLE 1
Biochemical activity data compounds of formula (I)

| | JNK3/SAPK1b, | | JNK2α2/SAPK1c, | | JNK1α1/SAP1α, | |
|---|---|---|---|---|---|---|
| | Active | | active | | Active | |
| COMPOUND | Mean | | Mean | | Mean | |
| ID | EC₅₀ (nM) | n= | EC₅₀ (nM) | n= | EC₅₀ (nM) | n= |
| SR-3306 | ** | n = 4 | ** | n = 2 | ** | n = 4 |
| Example I-9 | ** | n = 7 | ** | n = 2 | *** | n = 6 |
| Example I-10 | * | n = 9 | * | n = 2 | *** | n = 8 |
| Example I-11 | ** | n = 7 | ** | n = 1 | *** | n = 7 |
| Example I-22 | * | n = 6 | ** | n = 2 | *** | n = 6 |
| Example I-32 | * | n = 4 | * | n = 1 | | |
| Example I-86 | * | n = 5 | ** | n = 2 | *** | n = 5 |
| Example I-89 | * | n = 5 | * | n = 1 | *** | n = 5 |
| Example I-92 | * | n = 3 | * | n = 1 | ** | n = 3 |
| Example I-98 | * | n = 4 | * | n = 2 | ** | n = 3 |
| Example I-101 | * | n = 4 | * | n = 2 | *** | n = 4 |
| Example 1-51 | * | n = 4 | * | n = 1 | ** | n = 4 |
| Example I-104 | * | n = 2 | * | n = 1 | *** | n = 1 |

Biochemical EC₅₀s for inhibition of JNK1, JNK2 and JNK3 for JNK inhibitors.
SR-3306 and SP600125 are used as controls for the assay, n; number of experimental repeats. NI; no inhibition, SE; standard error
* <200 nM;
** 200-1000 nM;
*** >1000 nM

Table 2, below, provides biochemical activity data for modulators of JNK1, JNK2, and JNK3 of formula (II).

TABLE 2
JNK activity (IC₅₀) data for selected examples
of compounds of formula (II).

| | JNK3 (nM) | JNK1 (nM) |
|---|---|---|
| Example II-4 | * | * |
| Example II-5 | * | * |
| Example II-6 | *** | ** |
| Example II-8 | * | * |
| Example II-10 | * | * |
| Example II-11 | * | * |
| Example II-12 | * | * |
| Example II-14 | * | * |
| Example II-20 | * | * |
| Example II-21 | ** | ** |
| Example II-22 | * | * |
| Example II-23 | * | * |
| Example II-28 | ** | ** |
| Example II-29 | * | * |
| Example II-37 | ** | ** |
| Example II-40 | — | *** |
| Example II-42 | — | *** |
| Example II-49 | — | *** |

* IC₅₀ <200 nM;
** 200 nM < IC₅₀ <1000 nM;
*** IC₅₀ >1000 nM

### Evaluations

It is within ordinary skill to evaluate any compound disclosed and claimed herein for effectiveness in inhibition of JNK2 or JNK3 and in the various cellular assays using the procedures described in the Examples or found in the scientific literature. Accordingly, the person of ordinary skill can prepare and evaluate any of the claimed compounds without undue experimentation.

Any compound found to be an effective and selective inhibitor of JNK2, JNK3, or both, can likewise be tested in animal models and in human clinical studies using the skill and experience of the investigator to guide the selection of dosages and treatment regimens.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### EXAMPLES

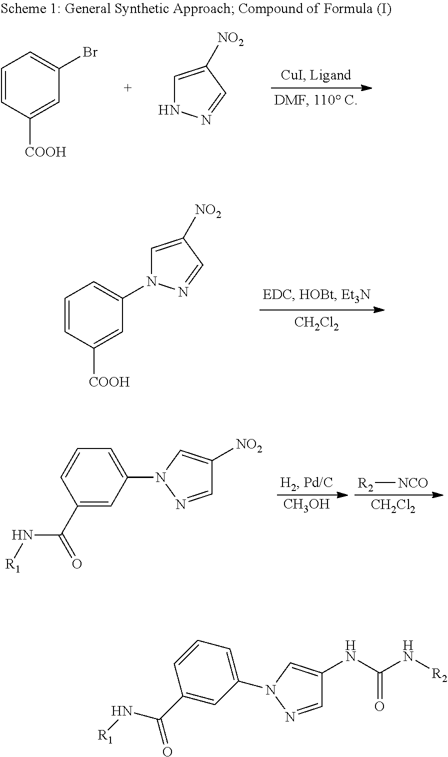

A mixture of 4-nitro-1H-pyrazole (10 mmol), 3-bromobenzoic acid (20 mmol), CuI (2.0 mmol), trans-N,N-dimethylcyclohexane-1,2-diamine (4.0 mmol) and Cs₂CO₃ (30 mmol) in DMF (20 mL) was purged with argon and stirred for 12 h at 100° C. in a sealed tube. The reaction mixture was cooled, and filtered through a pad of silica gel and rinsed with EtOAc. The resulting solution was concentrated in vacuo to yield a crude residue which was purified by chromatography on silica gel (EtOAc/hexane) to provide 3-(4-nitro-1H-pyrazol-1-yl)benzoic acid.

A solution of 3-(4-nitro-1H-pyrazol-1-yl)benzoic acid (5.0 mmol) in CH₂Cl₂ (20 mL) was added EDC (10 mmol), HOBt (10 mmol) and diisopropyl ethyl amine (15 mmol) and stirred for 30 min. Then the 6-methylpyridin-3-amine (5.5 mmol) was added and the resulting mixture was stirred over night. Water (50 ml) was added to the reaction mixture and extracted with EtOAc (2×100 mL). The resulting solution was concentrated in vacuo to yield a crude product.

This intermediate was hydrogenated in anhydrous methanol (100 mL) in the presence of 5% Pt—C (1.0 g) under a balloon of hydrogen for 3 hour. The mixture was filtered through a Celite pad and evaporated. The residue was purified by chromatography on silica gel (dichloromethane/methanol) to give the 3-(4-amino-1H-pyrazol-1-yl)-N-(6-methylpyridin-3-yl)benzamide.

1-chloro-2-isocyanatobenzene (0.12 mmol) was added to 3-(4-amino-1H-pyrazol-1-yl)-N-(6-methylpyridin-3-yl)benzamide (0.1 mmol) in CH₂Cl₂ (1.0 mL) at RT and stirred for 1 h. The solvent was evaporated and the residue was purified by reverse-phase preparative HPLC to give 3-(4-(3-(2-chlorophenyl) ureido)-1H-pyrazol-1-yl)-N-(6-methylpyridin-3-yl) benzamide. LC-MS: 447 (M+H). ¹H NMR (400 MHz, DMSO) δ 10.88 (s, 1H), 9.45 (s, 1H), 9.04 (s, 1H), 8.62 (s, 1H), 8.40 (s, 3H), 8.20 (dd, J=8.3, 1.5 Hz, 1H), 8.09 (d, J=8.2 Hz, 1H), 7.94-7.81 (m, 2H), 7.69 (t, J=8.0 Hz, 1H), 7.63 (s, 1H), 7.47 (dd, J=8.0, 1.4 Hz, 1H), 7.38-7.25 (m, 1H), 7.10-6.96 (m, 1H), 2.59 (s, 3H).

### Example I-1

### N-(4-chloro-3-fluorophenyl)-3-(4-(3-(2-chlorophenyl)ureido)-1H-pyrazol-1-yl)benzamide

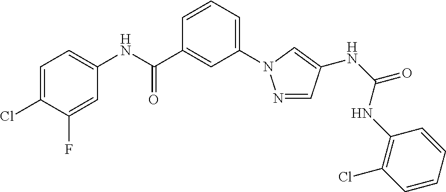 [SMILES: O=C(Nc1cnn(-c2cccc(C(=O)Nc3ccc(Cl)c(F)c3)c2)c1)Nc1ccccc1Cl]

Procedures in Scheme 1 were utilized to synthesize this compound. ¹H NMR (400 MHz, DMSO) δ 10.65 (s, 1H), 9.36 (s, 1H), 8.54 (s, 1H), 8.29 (dd, J=9.3, 7.4 Hz, 2H), 8.14 (dd, J=8.3, 1.5 Hz, 1H), 7.98 (d, J=1.4 Hz, 1H), 7.91 (dd, J=12.0, 2.1 Hz, 1H), 7.78 (dd, J=6.8, 4.3 Hz, 2H), 7.63-7.49 (m, 3H), 7.40 (dd, J=8.0, 1.5 Hz, 1H), 7.24 (s, 1H), 6.98 (dd, J=7.7, 1.3 Hz, 1H). LC-MS: 484 (M+H).

### Example I-2

### 3-(4-(3-(2-chlorophenyl)ureido)-1H-pyrazol-1-yl)-N-(3-methoxybenzyl)benzamide

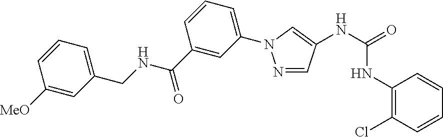 [SMILES: COc1cccc(CNC(=O)c2cccc(-n3cc(NC(=O)Nc4ccccc4Cl)cn3)c2)c1]

Procedures in Scheme 1 were utilized to synthesize this compound. ¹H NMR (400 MHz, DMSO) δ 9.41 (s, 1H), 9.24 (t, J=6.1 Hz, 1H), 8.58 (s, 1H), 8.38 (s, 1H), 8.30 (t, J=1.8 Hz, 1H), 8.20 (dd, J=8.3, 1.5 Hz, 1H), 8.02-7.95 (m, 1H), 7.81 (dd, J=6.6, 4.3 Hz, 2H), 7.60 (t, J=7.9 Hz, 1H), 7.47 (dd, J=8.0, 1.5 Hz, 1H), 7.33-7.22 (m, 2H), 7.08-6.99 (m, 1H), 6.92 (d, J=7.8 Hz, 2H), 6.86-6.79 (m, 1H), 4.50 (d, J=5.9 Hz, 2H), 3.74 (d, J=2.7 Hz, 3H). LC-MS: 476 (M+H).

### Example I-3

### 3-(4-(3-(2-chlorophenyl)ureido)-1H-pyrazol-1-yl)-N-(3-fluoro-4-methoxybenzyl)benzamide

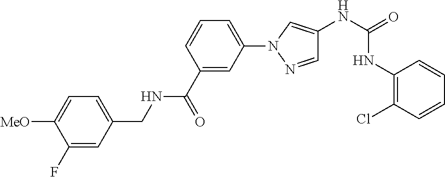 [SMILES: COc1ccc(CNC(=O)c2cccc(-n3cc(NC(=O)Nc4ccccc4Cl)cn3)c2)cc1F]

Procedures in Scheme 1 were utilized to synthesize this compound. ¹H NMR (400 MHz, DMSO) δ 9.41 (s, 1H), 9.22 (t, J=6.1 Hz, 1H), 8.58 (s, 1H), 8.37 (s, 1H), 8.31-8.26 (m, 1H), 8.20 (dd, J=8.3, 1.5 Hz, 1H), 7.99 (dd, J=8.1, 1.3 Hz, 1H), 7.82 (d, J=0.5 Hz, 1H), 7.79 (d, J=8.1 Hz, 1H), 7.59 (t, J=7.9 Hz, 1H), 7.47 (dd, J=8.0, 1.4 Hz, 1H), 7.34-7.27 (m, 1H), 7.22-7.15 (m, 1H), 7.13 (t, J=3.4 Hz, 2H), 7.07-7.00 (m, 1H), 4.45 (d, J=5.9 Hz, 2H), 3.82 (s, 3H). LC-MS: 494 (M+H).

### Example I-4

### N-benzyl-3-(4-(3-(2-chlorophenyl)ureido)-1H-pyrazol-1-yl)-N-methylbenzamide

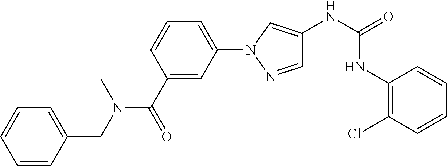 [SMILES: CN(Cc1ccccc1)C(=O)c1cccc(-n2cc(NC(=O)Nc3ccccc3Cl)cn2)c1]

Procedures in Scheme 1 were utilized to synthesize this compound. ¹H NMR (400 MHz, DMSO) δ 9.38 (s, 1H), 8.56 (s, 1H), 8.37 (s, 1H), 8.21 (dd, J=8.3, 1.4 Hz, 1H), 7.87 (d, J=18.5 Hz, 3H), 7.57 (s, 1H), 7.47 (dd, J=8.0, 1.4 Hz, 1H), 7.39 (s, 4H), 7.34-7.26 (m, 2H), 7.22 (s, 1H), 7.04 (td, J=7.7, 1.5 Hz, 1H), 4.71 (s, 1H), 4.52 (s, 1H), 2.89 (d, J=23.4 Hz, 3H). LC-MS: 460 (M+H).

### Example I-5

### N-benzyl-3-(4-(3-(2-chlorophenyl)ureido)-1H-pyrazol-1-yl)-N-(2-(dimethylamino)ethyl)benzamide

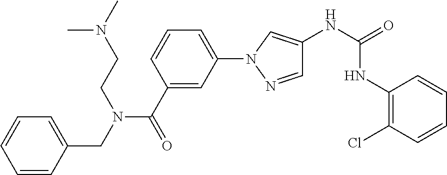 [SMILES: CN(C)CCN(Cc1ccccc1)C(=O)c1cccc(-n2cc(NC(=O)Nc3ccccc3Cl)cn2)c1]

Procedures in Scheme 1 were utilized to synthesize this compound. LC-MS: 517 (M+H).

### Example I-6

### N-benzyl-3-(4-(3-(2-chlorophenyl)ureido)-1H-pyrazol-1-yl)-N-(2-hydroxyethyl)benzamide

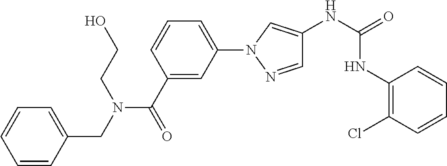 [SMILES: O=C(Nc1cnn(-c2cccc(C(=O)N(CCO)Cc3ccccc3)c2)c1)Nc1ccccc1Cl]

Procedures in Scheme 1 were utilized to synthesize this compound. LC-MS: 490 (M+H).

### Example I-7

### 3-(4-(3-(2-chlorophenyl)ureido)-1H-pyrazol-1-yl)-N-(3,5-difluorophenyl)benzamide

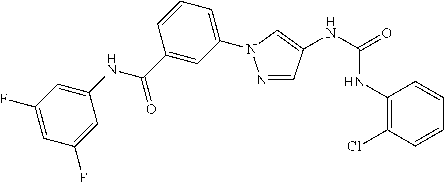 [SMILES: O=C(Nc1cnn(-c2cccc(C(=O)Nc3cc(F)cc(F)c3)c2)c1)Nc1ccccc1Cl]

Procedures in Scheme 1 were utilized to synthesize this compound. LC-MS: 468 (M+H).

### Example I-8

### 2-fluoro-5-(4-(3-phenylureido)-1H-pyrazol-1-yl)-N-(3,4,5-trimethoxyphenyl)benzamide

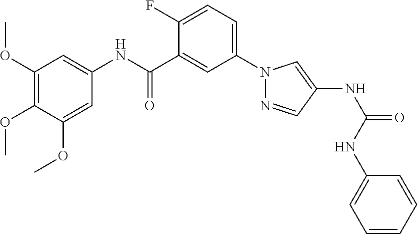 [SMILES: COc1cc(NC(=O)c2cc(-n3cc(NC(=O)Nc4ccccc4)cn3)ccc2F)cc(OC)c1OC]

Procedures in Scheme 1 were utilized to synthesize this compound. ¹H NMR (400 MHz, DMSO) δ 10.47 (s, 1H), 9.40 (s, 1H), 8.55 (s, 1H), 8.37 (s, 1H), 8.20 (d, J=6.8 Hz, 1H), 8.07 (d, J=5.8 Hz, 1H), 8.02 (s, 1H), 7.85 (s, 1H), 7.52-7.44 (m, 2H), 7.30 (t, J=8.6 Hz, 1H), 7.16 (s, 2H), 7.07-6.99 (m, 1H), 3.78 (s, 6H), 3.65 (s, 3H). LC-MS: 506 (M+H).

### Example I-9

### 3-(4-(3-(2-chlorophenyl)ureido)-1H-pyrazol-1-yl)-N-((6-methylpyridin-3-yl)methyl)benzamide

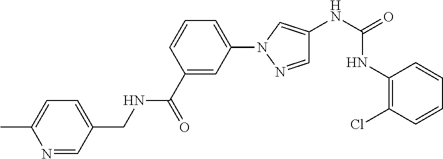 [SMILES: Cc1ccc(CNC(=O)c2cccc(-n3cc(NC(=O)Nc4ccccc4Cl)cn3)c2)cn1]

Procedures in Scheme 1 were utilized to synthesize this compound. LC-MS: 461 (M+H).

### Example I-10

### 3-(4-(3-(2-chlorophenyl)ureido)-1H-pyrazol-1-yl)-N-(2-methylpyridin-4-yl)benzamide

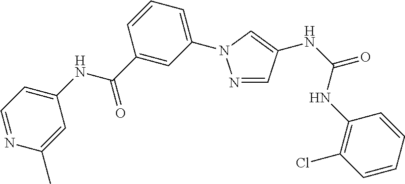 [SMILES: Cc1cc(NC(=O)c2cccc(-n3cc(NC(=O)Nc4ccccc4Cl)cn3)c2)ccn1]

Procedures in Scheme 1 were utilized to synthesize this compound. LC-MS: 447 (M+H).

### Example I-11

### 3-(4-(3-(2-chlorophenyl)ureido)-1H-pyrazol-1-yl)-N-((6-methylpyridin-2-yl)methyl)benzamide

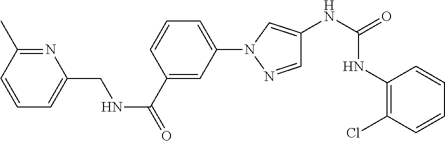 [SMILES: Cc1cccc(CNC(=O)c2cccc(-n3cc(NC(=O)Nc4ccccc4Cl)cn3)c2)n1]

Procedures in Scheme 1 were utilized to synthesize this compound. ¹H NMR (400 MHz, DMSO) δ 9.46 (d, J=8.4 Hz, 2H), 8.60 (s, 1H), 8.41 (s, 1H), 8.34 (s, 1H), 8.19 (dd, J=8.3, 1.4 Hz, 1H), 8.02 (dt, J=7.1, 3.5 Hz, 2H), 7.82 (d, J=5.8 Hz, 2H), 7.62 (t, J=7.9 Hz, 1H), 7.50-7.41 (m, 3H), 7.34-7.25 (m, 1H), 7.07-6.99 (m, 1H), 4.68 (d, J=5.6 Hz, 2H), 2.61 (s, 3H). LC-MS: 461 (M+H).

### Example I-12

### 3-(4-(3-(2-chlorophenyl)ureido)-1H-pyrazol-1-yl)-N-(6-methylpyridin-3-yl)benzamide

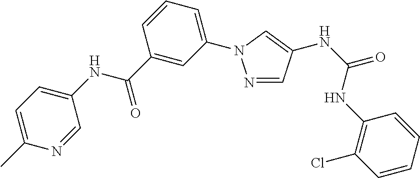 [SMILES: Cc1ccc(NC(=O)c2cccc(-n3cc(NC(=O)Nc4ccccc4Cl)cn3)c2)cn1]

Procedures in Scheme 1 were utilized to synthesize this compound. ¹H NMR (400 MHz, DMSO) δ 10.88 (s, 1H), 9.45 (s, 1H), 9.04 (s, 1H), 8.62 (s, 1H), 8.40 (s, 3H), 8.20 (dd, J=8.3, 1.5 Hz, 1H), 8.09 (d, J=8.2 Hz, 1H), 7.94-7.81 (m, 2H), 7.69 (t, J=8.0 Hz, 1H), 7.63 (s, 1H), 7.47 (dd, J=8.0, 1.4 Hz, 1H), 7.38-7.25 (m, 1H), 7.10-6.96 (m, 1H), 2.59 (s, 3H). LC-MS: 447 (M+H).

### Example I-13

### 5-(4-(3-(2-chlorophenyl)ureido)-1H-pyrazol-1-yl)-2-fluoro-N-(5-methylpyridin-3-yl)benzamide

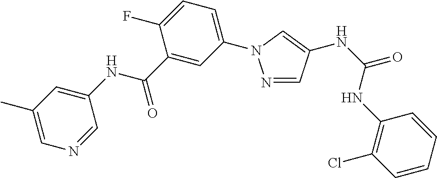 [SMILES: Cc1cncc(NC(=O)c2cc(-n3cc(NC(=O)Nc4ccccc4Cl)cn3)ccc2F)c1]

Procedures in Scheme 1 were utilized to synthesize this compound. ¹H NMR (400 MHz, DMSO) δ 10.93 (s, 1H), 9.41 (s, 1H), 8.85 (s, 1H), 8.57 (s, 1H), 8.38 (s, 1H), 8.34 (s, 1H), 8.16 (ddd, J=8.7, 7.1, 2.2 Hz, 3H), 8.05 (d, J=9.1 Hz, 1H), 7.85 (s, 1H), 7.53 (t, J=9.3 Hz, 1H), 7.47 (dd, J=8.0, 1.4 Hz, 1H), 7.30 (t, J=7.1 Hz, 1H), 7.07-6.98 (m, 1H), 2.40 (s, 3H). LC-MS: 466 (M+H).

### Example I-14

### 3-(4-(3-(2-chlorophenyl)ureido)-1H-pyrazol-1-yl)-N-(1-methyl-1H-indazol-5-yl)benzamide

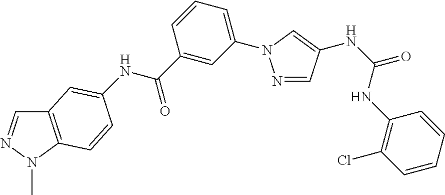 [SMILES: Cn1ncc2cc(NC(=O)c3cccc(-n4cc(NC(=O)Nc5ccccc5Cl)cn4)c3)ccc21]

Procedures in Scheme 1 were utilized to synthesize this compound. LC-MS: 487 (M+H).

### Example I-15

### 5-(4-(3-(2-chlorophenyl)ureido)-1H-pyrazol-1-yl)-2-fluoro-N-(1-methyl-1H-indazol-5-yl)benzamide

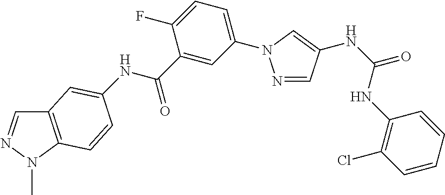 [SMILES: Cn1ncc2cc(NC(=O)c3cc(-n4cc(NC(=O)Nc5ccccc5Cl)cn4)ccc3F)ccc21]

Procedures in Scheme 1 were utilized to synthesize this compound. LC-MS: 505 (M+H).

### Example I-16

### 5-(4-(3-(2-chlorophenyl)ureido)-1H-pyrazol-1-yl)-2-fluoro-N-(2-methylpyridin-4-yl)benzamide

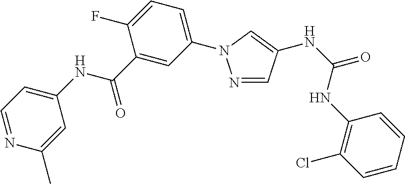 [SMILES: Cc1cc(NC(=O)c2cc(-n3cc(NC(=O)Nc4ccccc4Cl)cn3)ccc2F)ccn1]

Procedures in Scheme 1 were utilized to synthesize this compound. ¹H NMR (400 MHz, DMSO) δ 11.66 (s, 1H), 9.44 (s, 1H), 8.64 (d, J=6.5 Hz, 1H), 8.58 (s, 1H), 8.39 (s, 1H), 8.19 (dd, J=6.5, 1.9 Hz, 2H), 8.15-8.06 (m, 1H), 8.04 (s, 1H), 7.94 (d, J=6.3 Hz, 1H), 7.85 (s, 1H), 7.57 (t, J=9.3 Hz, 1H), 7.47 (dd, J=8.0, 1.5 Hz, 1H), 7.32-7.24 (m, 1H), 7.09-6.97 (m, 1H), 2.67 (s, 3H). LC-MS: 466 (M+H).

### Example I-17

### 5-(4-(3-(2-chlorophenyl)ureido)-1H-pyrazol-1-yl)-2-fluoro-N-(6-methylpyridin-3-yl)benzamide

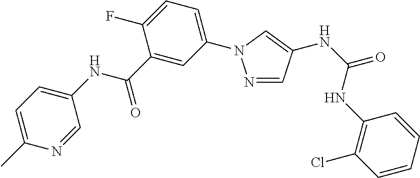 [SMILES: Cc1ccc(NC(=O)c2cc(-n3cc(NC(=O)Nc4ccccc4Cl)cn3)ccc2F)cn1]

Procedures in Scheme 1 were utilized to synthesize this compound. ¹H NMR (400 MHz, DMSO) δ 10.90 (s, 1H), 9.41 (s, 1H), 8.91 (s, 1H), 8.57 (s, 1H), 8.38 (s, 1H), 8.19 (d, J=6.8 Hz, 2H), 8.14 (s, 1H), 8.04 (s, 1H), 7.85 (s, 1H), 7.56-7.42 (m, 3H), 7.30 (t, J=7.8 Hz, 1H), 7.10-6.97 (m, 1H), 2.54 (s, 3H). LC-MS: 466 (M+H).

### Example I-18

### 1-(2-chlorophenyl)-3-(1-(4-fluoro-3-(3H-imidazo[4,5-c]pyridin-2-yl)phenyl)-1 H-pyrazol-4-yl)urea

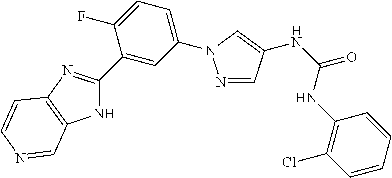 [SMILES: O=C(Nc1cnn(-c2ccc(F)c(-c3nc4ccncc4[nH]3)c2)c1)Nc1ccccc1Cl]

Procedures in Scheme 1 were utilized to synthesize this compound. LC-MS: 449 (M+H).

### Example I-19

### 1-(1-(3-(3H-imidazo[4,5-c]pyridin-2-yl)phenyl)-1H-pyrazol-4-yl)-3-(2-chlorophenyl)urea

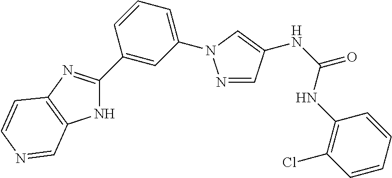 [SMILES: O=C(Nc1cnn(-c2cccc(-c3nc4ccncc4[nH]3)c2)c1)Nc1ccccc1Cl]

Procedures in Scheme 1 were utilized to synthesize this compound. LC-MS: 431 (M+H).

### Example I-20

### 5-(4-(3-(2-chlorophenyl)ureido)-1H-pyrazol-1-yl)-2-fluoro-N-(2-methylpyrimidin-5-yl)benzamide

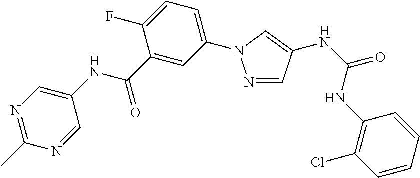 [SMILES: Cc1ncc(NC(=O)c2cc(-n3cc(NC(=O)Nc4ccccc4Cl)cn3)ccc2F)cn1]

Procedures in Scheme 1 were utilized to synthesize this compound. ¹H NMR (400 MHz, DMSO) δ 10.86 (s, 1H), 9.41 (s, 1H), 9.03 (s, 2H), 8.57 (s, 1H), 8.37 (s, 1H), 8.19 (d, J=8.3 Hz, 2H), 8.08-8.01 (m, 1H), 7.85 (s, 1H), 7.53 (s, 1H), 7.46 (d, J=8.1 Hz, 1H), 7.33-7.26 (m, 1H), 7.03 (s, 1H), 2.62 (s, 3H). LC-MS: 467 (M+H).

### Example I-21

### 3-(4-(3-(2-chlorophenyl)ureido)-1H-pyrazol-1-yl)-N-(2-methylpyrimidin-5-yl)benzamide

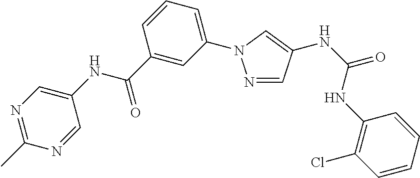 [SMILES: Cc1ncc(NC(=O)c2cccc(-n3cc(NC(=O)Nc4ccccc4Cl)cn3)c2)cn1]

Procedures in Scheme 1 were utilized to synthesize this compound. ¹H NMR (400 MHz, DMSO) δ 10.74 (s, 1H), 9.44 (s, 1H), 9.08 (s, 2H), 8.61 (s, 1H), 8.40 (d, J=5.1 Hz, 2H), 8.21 (d, J=8.2 Hz, 1H), 8.08 (d, J=7.7 Hz, 1H), 7.88 (d, J=12.3 Hz, 2H), 7.68 (t, J=7.9 Hz, 1H), 7.47 (d, J=7.8 Hz, 1H), 7.31 (t, J=7.5 Hz, 1H), 7.04 (t, J=7.3 Hz, 1H), 2.62 (s, 3H). LC-MS: 449 (M+H).

### Example I-22

### 3-(4-(3-(2-chlorophenyl)ureido)-1H-pyrazol-1-yl)-N-(2,6-dimethylpyridin-4-yl)benzamide

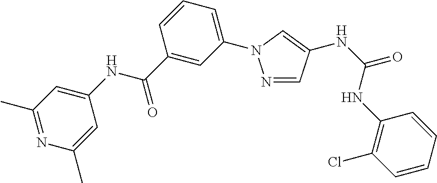 [SMILES: Cc1cc(NC(=O)c2cccc(-n3cc(NC(=O)Nc4ccccc4Cl)cn3)c2)cc(C)n1]

Procedures in Scheme 1 were utilized to synthesize this compound. LC-MS: 462 (M+H).

### Example I-23

### 5-(4-(3-(2-chlorophenyl)ureido)-1H-pyrazol-1-yl)-N-(2,6-dimethylpyridin-4-yl)-2-fluorobenzamide

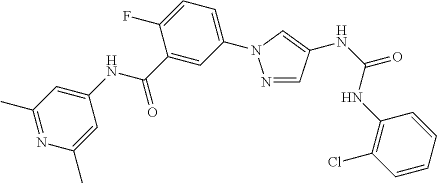 [SMILES: Cc1cc(NC(=O)c2cc(-n3cc(NC(=O)Nc4ccccc4Cl)cn3)ccc2F)cc(C)n1]

Procedures in Scheme 1 were utilized to synthesize this compound. LC-MS: 480 (M+H).

### Example I-24

### 5-(4-(3-(2-chlorophenyl)ureido)-1H-pyrazol-1-yl)-2-fluoro-N-((6-methylpyridin-3-yl)methyl)benzamide

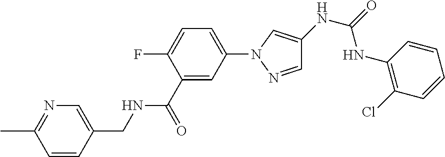 [SMILES: Cc1ccc(CNC(=O)c2cc(-n3cc(NC(=O)Nc4ccccc4Cl)cn3)ccc2F)cn1]

Procedures in Scheme 1 were utilized to synthesize this compound. LC-MS: 479 (M+H).

### Example I-25

### 3-(4-(3-(2-chlorophenyl)ureido)-1H-pyrazol-1-yl)-N-(6-methylpyridin-2-yl)benzamide

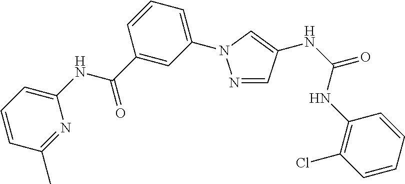 [SMILES: Cc1cccc(NC(=O)c2cccc(-n3cc(NC(=O)Nc4ccccc4Cl)cn3)c2)n1]

Procedures in Scheme 1 were utilized to synthesize this compound. LC-MS: 447 (M+H).

### Example I-26

### 3-(4-(3-(2-chlorophenyl)ureido)-1H-pyrazol-1-yl)-N-(2-methylpyridin-3-yl)benzamide

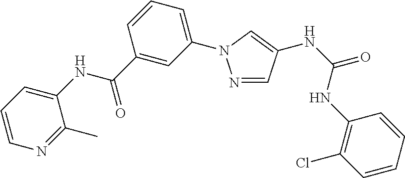 [SMILES: Cc1ncccc1NC(=O)c1cccc(-n2cc(NC(=O)Nc3ccccc3Cl)cn2)c1]

Procedures in Scheme 1 were utilized to synthesize this compound. LC-MS: 447 (M+H).

### Example I-27

### 3-(4-(3-(2-chlorophenyl)ureido)-1H-pyrazol-1-yl)-N-(5-methylpyridin-2-yl)benzamide

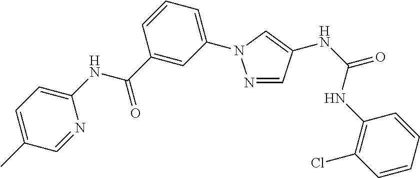 [SMILES: Cc1ccc(NC(=O)c2cccc(-n3cc(NC(=O)Nc4ccccc4Cl)cn3)c2)nc1]

Procedures in Scheme 1 were utilized to synthesize this compound. ¹H NMR (400 MHz, DMSO) δ 11.07 (s, 1H), 9.48 (s, 1H), 8.70 (s, 1H), 8.41 (s, 2H), 8.26 (s, 1H), 8.25-8.19 (m, 1H), 8.13 (d, J=8.3 Hz, 1H), 8.06 (d, J=8.3 Hz, 1H), 7.91 (d, J=7.6 Hz, 1H), 7.82 (s, 1H), 7.73 (d, J=7.7 Hz, 1H), 7.63 (t, J=7.9 Hz, 1H), 7.47 (dd, J=8.0, 1.4 Hz, 1H), 7.31 (t, J=8.5 Hz, 1H), 7.09-7.00 (m, 1H), 2.31 (s, 4H). LC-MS: 447 (M+H).

### Example I-28

### 3-(4-(3-(2-chlorophenyl)ureido)-1H-pyrazol-1-yl)-N-methyl-N-(6-methylpyridin-3-yl)benzamide

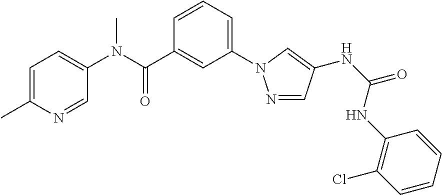 [SMILES: Cc1ccc(N(C)C(=O)c2cccc(-n3cc(NC(=O)Nc4ccccc4Cl)cn3)c2)cn1]

Procedures in Scheme 1 were utilized to synthesize this compound. LC-MS: 461 (M+H).

### Example I-29

### 3-(4-(3-(2-chlorophenyl)ureido)-1H-pyrazol-1-yl)-N-(2-methyl-2H-indazol-5-yl)benzamide

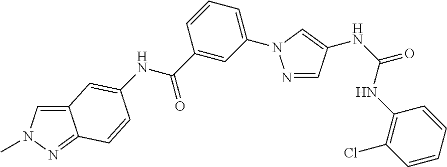 [SMILES: Cn1cc2cc(NC(=O)c3cccc(-n4cc(NC(=O)Nc5ccccc5Cl)cn4)c3)ccc2n1]

Procedures in Scheme 1 were utilized to synthesize this compound. LC-MS: 486 (M+H).

### Example I-30

### 3-(4-(3-(2-chlorophenyl)ureido)-1H-pyrazol-1-yl)-N-(2,6-dimethylpyridin-3-yl)benzamide

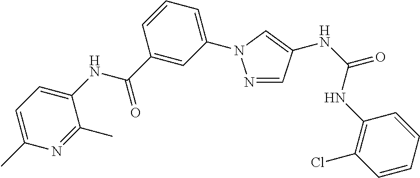 [SMILES: Cc1ccc(NC(=O)c2cccc(-n3cc(NC(=O)Nc4ccccc4Cl)cn3)c2)c(C)n1]

¹H NMR (400 MHz, DMSO) δ 10.56 (s, 1H), 9.46 (s, 1H), 8.62 (s, 1H), 8.46-8.33 (m, 2H), 8.19 (dd, J=8.3, 1.5 Hz, 2H), 8.09 (dd, J=8.1, 1.4 Hz, 1H), 7.89 (d, J=8.0 Hz, 1H), 7.85 (s, 1H), 7.68 (t, J=7.9 Hz, 1H), 7.57 (s, 1H), 7.47 (dd, J=8.0, 1.5 Hz, 1H), 7.34-7.27 (m, 1H), 7.09-7.00 (m, 1H), 2.63 (s, 3H), 2.57 (s, 3H).

Procedures in Scheme 1 were utilized to synthesize this compound. LC-MS: 462 (M+H).

### Example I-31

### 3-(4-(3-(2-chlorophenyl)ureido)-1H-pyrazol-1-yl)-N-(2-oxoindolin-5-yl)benzamide

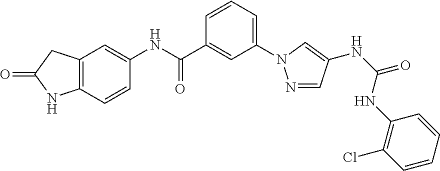 [SMILES: O=C1Cc2cc(NC(=O)c3cccc(-n4cc(NC(=O)Nc5ccccc5Cl)cn4)c3)ccc2N1]

Procedures in Scheme 1 were utilized to synthesize this compound. ¹H NMR (400 MHz, DMSO) δ 10.37 (s, 1H), 10.31 (s, 1H), 9.42 (s, 1H), 8.61 (s, 1H), 8.38 (s, 1H), 8.33 (t, J=1.8 Hz, 1H), 8.21 (dd, J=8.3, 1.5 Hz, 1H), 8.02 (dd, J=8.1, 1.3 Hz, 1H), 7.84 (d, J=8.9 Hz, 2H), 7.69 (s, 1H), 7.64 (t, J=7.9 Hz, 1H), 7.54 (dd, J=8.4, 2.0 Hz, 1H), 7.48-7.42 (m, 1H), 7.36-7.26 (m, 1H), 7.10-6.95 (m, 1H), 6.82 (d, J=8.3 Hz, 1H), 3.52 (s, 2H).

LC-MS: 487 (M+H).

### Example I-32

### 3-(4-(3-(2-chlorophenyl)ureido)-1H-pyrazol-1-yl)-4-fluoro-N-(6-methylpyridin-3-yl)benzamide

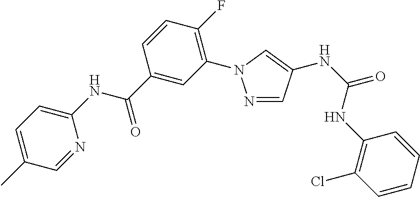 [SMILES: Cc1ccc(NC(=O)c2ccc(F)c(-n3cc(NC(=O)Nc4ccccc4Cl)cn3)c2)nc1]

Procedures in Scheme 1 were utilized to synthesize this compound. ¹H NMR (400 MHz, DMSO) δ 10.92 (s, 1H), 9.46 (s, 1H), 9.05 (s, 1H), 8.50 (dd, J=7.5, 2.3 Hz, 1H), 8.43-8.36 (m, 2H), 8.18 (dd, J=8.3, 1.4 Hz, 1H), 8.06-7.97 (m, 1H), 7.91 (s, 1H), 7.70 (dd, J=11.5, 8.7 Hz, 1H), 7.65 (s, 1H), 7.47 (dd, J=8.0, 1.4 Hz, 1H), 7.35-7.25 (m, 1H), 7.14-6.88 (m, 1H), 2.60 (s, 3H).

LC-MS: 464 (M+H).

### Example I-33

### 3-(4-(3-(2-chlorophenyl)ureido)-1H-pyrazol-1-yl)-N-(1,5-naphthyridin-4-yl)benzamide

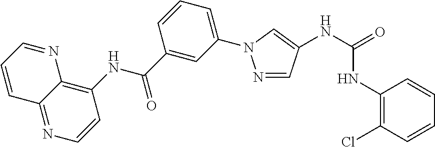 [SMILES: O=C(Nc1cnn(-c2cccc(C(=O)Nc3ccnc4cccnc34)c2)c1)Nc1ccccc1Cl]

Procedures in Scheme 1 were utilized to synthesize this compound. LC-MS: 484 (M+H).

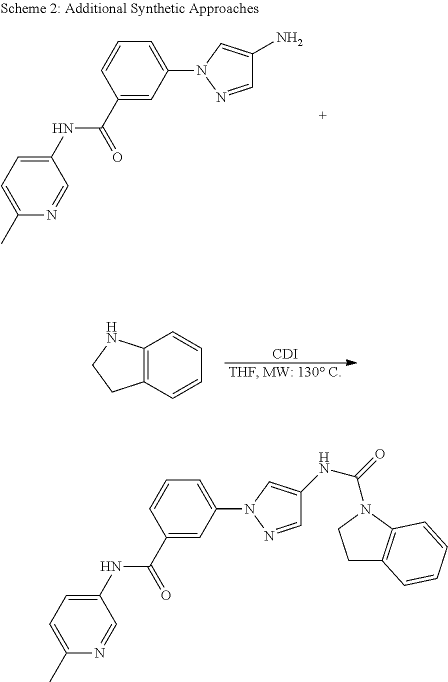 [SMILES: Cc1ccc(NC(=O)c2cccc(-n3cc(N)cn3)c2)cn1 ; c1ccc2c(c1)CCN2 ; "CDI" ; "THF, MW: 130° C." ; Cc1ccc(NC(=O)c2cccc(-n3cc(NC(=O)N4CCc5ccccc54)cn3)c2)cn1]

A mixture of 3-(4-amino-1H-pyrazol-1-yl)-N-(6-methylpyridin-3-yl)benzamide (0.1 mmol), Indoline (4 eq.) and CDI (4 eq.) in THF (1.0 mL) in a sealed tube was heated at 130° C. using microwave for 30 min. The solvent was evaporated and the residue was purified by reverse-phase preparative HPLC to give the product.

### Example I-34

### N-(1-(3-(pyridin-4-ylcarbamoyl)phenyl)-1H-pyrazol-4-yl)isoindoline-2-carboxamide

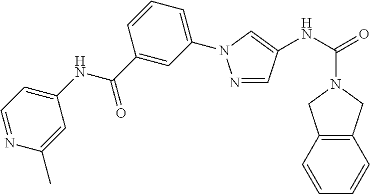 [SMILES: Cc1cc(NC(=O)c2cccc(-n3cc(NC(=O)N4Cc5ccccc5C4)cn3)c2)ccn1]

Procedures in Scheme 2 were utilized to synthesize this compound. LC-MS: 439 (M+H).

### Example I-35

### N-(1-(3-((6-methylpyridin-3-yl)carbamoyl)phenyl)-1H-pyrazol-4-yl)indoline-1-carboxamide

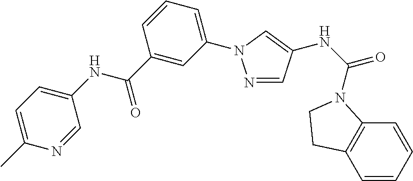 [SMILES: Cc1ccc(NC(=O)c2cccc(-n3cc(NC(=O)N4CCc5ccccc54)cn3)c2)cn1]

Procedures in Scheme 2 were utilized to synthesize this compound. LC-MS: 439 (M+H).

### Example I-36

### 3-(4-(3-(2-chlorophenyl)-3-methylureido)-1H-pyrazol-1-yl)-N-(6-methylpyridin-3-yl)benzamide

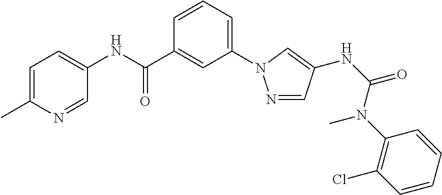 [SMILES: Cc1ccc(NC(=O)c2cccc(-n3cc(NC(=O)N(C)c4ccccc4Cl)cn3)c2)cn1]

Procedures in Scheme 2 were utilized to synthesize this compound. LC-MS: 461 (M+H).

### Example I-37

### 3-(4-(3-(2-chlorophenyl)ureido)-1H-pyrazol-1-yl)-2-fluoro-N-(6-methylpyridin-3-yl)benzamide

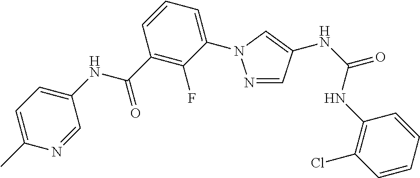 [SMILES: Cc1ccc(NC(=O)c2cccc(-n3cc(NC(=O)Nc4ccccc4Cl)cn3)c2F)cn1]

Procedures in Scheme 1 were utilized to synthesize this compound. LC-MS: 465 (M+H).

### Example I-38

### 3-(4-(3-(2-chlorophenyl)ureido)-1H-pyrazol-1-yl)-2-fluoro-N-(6-methylpyridin-3-yl)benzamide

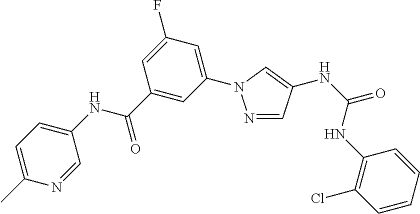 [SMILES: Cc1ccc(NC(=O)c2cc(F)cc(-n3cc(NC(=O)Nc4ccccc4Cl)cn3)c2)cn1]

Procedures in Scheme 1 were utilized to synthesize this compound. LC-MS: 465 (M+H).

### Example I-39

### 3-(4-(3-(2-chlorobenzyl)ureido)-1H-pyrazol-1-yl)-N-(6-methylpyridin-3-yl)benzamide

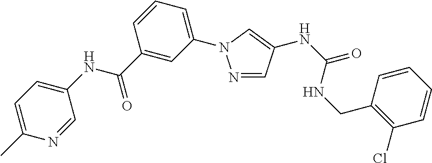 [SMILES: Cc1ccc(NC(=O)c2cccc(-n3cc(NC(=O)NCc4ccccc4Cl)cn3)c2)cn1]

Procedures in Scheme 2 were utilized to synthesize this compound. LC-MS: 461 (M+H).

### Example I-40

### 3-(4-(3-(2-chlorophenyl)-3-(2-hydroxyethyl)ureido)-1H-pyrazol-1-yl)-N-(6-methylpyridin-3-yl)benzamide

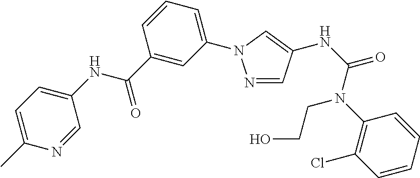 [SMILES: Cc1ccc(NC(=O)c2cccc(-n3cc(NC(=O)N(CCO)c4ccccc4Cl)cn3)c2)cn1]

Procedures in Scheme 2 were utilized to synthesize this compound. LC-MS: 491 (M+H).

### Example I-41

### N-(6-methylpyridin-3-yl)-3-(4-(3-(pyrrolidin-1-yl)ureido)-1H-pyrazol-1-yl)benzamide

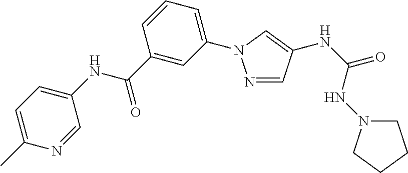 [SMILES: Cc1ccc(NC(=O)c2cccc(-n3cc(NC(=O)NN4CCCC4)cn3)c2)cn1]

Procedures in Scheme 2 were utilized to synthesize this compound. LC-MS: 406 (M+H).

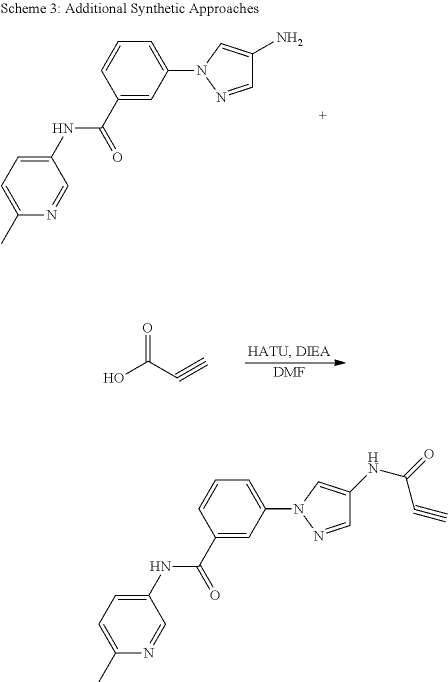

The acid (0.2 mmol) was added HATU (2 eq.) and diisopropyl ethyl amine (2 eq.) in DMF, stirred for 30 min. The 3-(4-amino-1H-pyrazol-1-yl)-N-(6-methylpyridin-3-yl)benzamide (0.1 mmol) was added to the mixture and the resulting mixture was stirred over night. The residue was purified by reverse-phase preparative HPLC to give the product.

### Example I-42

### 3-(4-(but-3-ynamido)-1H-pyrazol-1-yl)-N-(6-methylpyridin-3-yl)benzamide

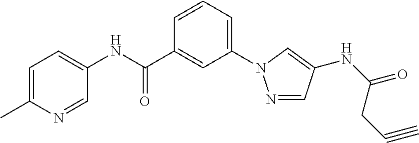 [SMILES: C#CCC(=O)Nc1cnn(-c2cccc(C(=O)Nc3ccc(C)nc3)c2)c1]

Procedures in Scheme 3 were utilized to synthesize this compound. LC-MS: 360 (M+H).

### Example I-43

### N-(6-methylpyridin-3-yl)-3-(4-(2-phenoxyacetamido)-1H-pyrazol-1-yl)benzamide

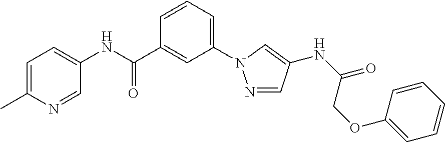 [SMILES: Cc1ccc(NC(=O)c2cccc(-n3cc(NC(=O)COc4ccccc4)cn3)c2)cn1]

Procedures in Scheme 3 were utilized to synthesize this compound. LC-MS: 428 (M+H).

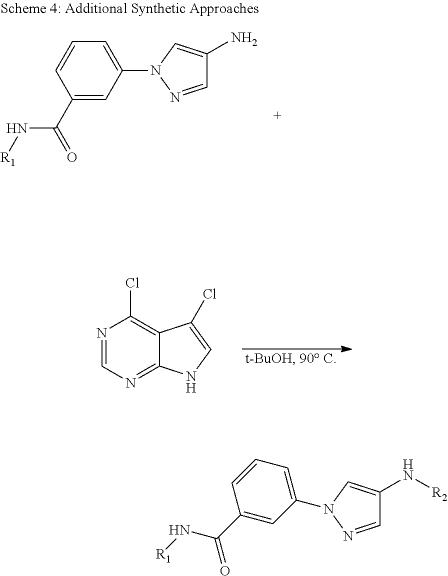

The 4,5-dichloro-7H-pyrrolo[2,3-d]pyrimidine (0.11 mmol) was added to 3-(4-amino-1H-pyrazol-1-yl)-N-(6-methylpyridin-3-yl)benzamide (0.1 mmol) in t-BuOH (1.0 mL) and stirred at 90° C. for 12 h. The solvent was evaporated and the residue was purified by reverse-phase preparative HPLC to give the product.

### Example I-44

### 3-(4-((5-chloro-7H-pyrrolo[2,3-d]pyrimidin-4-yl)amino)-1H-pyrazol-1-yl)-N-(6-methylpyridin-3-yl)benzamide

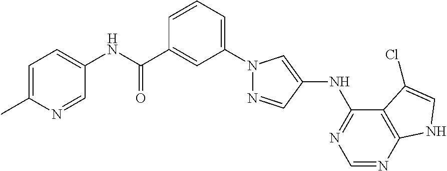 [SMILES: Cc1ccc(NC(=O)c2cccc(-n3cc(Nc4ncnc5[nH]cc(Cl)c45)cn3)c2)cn1]

Procedures in Scheme 4 were utilized to synthesize this compound. LC-MS: 445 (M+H).

### Example I-45

### 3-(4-(3-(2-chlorophenyl)ureido)-1H-pyrazol-1-yl)-N-(2-cyclopropyl-3-methyl-4-oxo-3,4-dihydroquinazolin-7-yl)benzamide

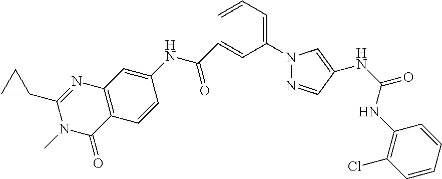 [SMILES: Cn1c(C2CC2)nc2cc(NC(=O)c3cccc(-n4cc(NC(=O)Nc5ccccc5Cl)cn4)c3)ccc2c1=O]

Procedures in Scheme 1 were utilized to synthesize this compound. LC-MS: 554 (M+H).

### Example I-46

### 3-(4-(3-(2-chlorophenyl)ureido)-1H-pyrazol-1-yl)-N-(3-methylcinnolin-6-yl)benzamide

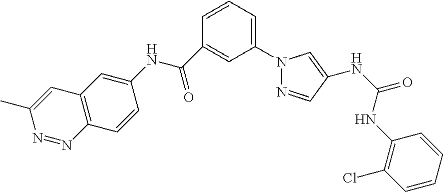 [SMILES: Cc1cc2cc(NC(=O)c3cccc(-n4cc(NC(=O)Nc5ccccc5Cl)cn4)c3)ccc2nn1]

Procedures in Scheme 1 were utilized to synthesize this compound. LC-MS: 498 (M+H).

### Example I-47

### 2-chlorobenzyl (1-(3-((6-methylpyridin-3-yl)carbamoyl)phenyl)-1H-pyrazol-4-yl)carbamate

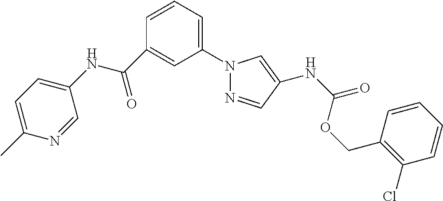 [SMILES: Cc1ccc(NC(=O)c2cccc(-n3cc(NC(=O)OCc4ccccc4Cl)cn3)c2)cn1]

Procedures in Scheme 1 were utilized to synthesize this compound. ¹H NMR (400 MHz, DMSO) δ 10.50 (s, 1H), 9.90 (s, 1H), 8.76 (d, J=2.4 Hz, 1H), 8.41 (s, 1H), 8.28 (s, 1H), 8.05 (dd, J=8.4, 2.6 Hz, 1H), 7.96 (d, J=7.4 Hz, 1H), 7.80 (d, J=8.2 Hz, 1H), 7.64 (s, 1H), 7.58 (t, J=8.0 Hz, 1H), 7.52-7.48 (m, 1H), 7.46 (dd, J=5.7, 3.6 Hz, 1H), 7.34 (dd, J=5.8, 3.5 Hz, 2H), 7.25 (d, J=8.4 Hz, 1H), 5.19 (s, 2H), 2.40 (s, 3H). LC-MS: 462 (M+H).

### Example I-48

### 2-chlorophenyl (1-(3-((6-methylpyridin-3-yl)carbamoyl)phenyl)-1H-pyrazol-4-yl)carbamate

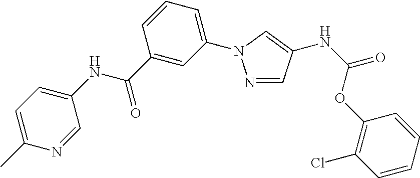 [SMILES: Cc1ccc(NC(=O)c2cccc(-n3cc(NC(=O)Oc4ccccc4Cl)cn3)c2)cn1]

Procedures in Scheme 1 were utilized to synthesize this compound. ¹H NMR (400 MHz, DMSO) δ 10.50 (d, J=5.5 Hz, 2H), 8.76 (d, J=2.4 Hz, 1H), 8.45 (s, 1H), 8.33-8.25 (m, 1H), 8.05 (dd, J=8.3, 2.5 Hz, 1H), 8.01-7.93 (m, 1H), 7.81 (d, J=8.2 Hz, 1H), 7.72 (s, 1H), 7.59 (t, J=7.9 Hz, 1H), 7.54 (d, J=7.8 Hz, 1H), 7.36 (dd, J=5.0, 1.1 Hz, 2H), 7.31-7.20 (m, 2H), 2.41 (d, J=3.1 Hz, 3H). LC-MS: 448 (M+H).

### Example I-49

### 2-methoxyphenyl(1-(3-((6-methylpyridin-3-yl)carbamoyl)phenyl)-1H-pyrazol-4-yl)carbamate

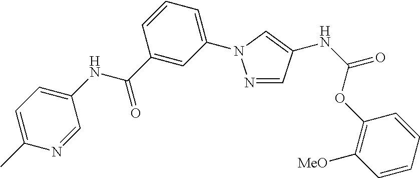 [SMILES: COc1ccccc1OC(=O)Nc1cnn(-c2cccc(C(=O)Nc3ccc(C)nc3)c2)c1]

Procedures in Scheme 1 were utilized to synthesize this compound. LC-MS: 444 (M+H).

### Example I-50

### 3-(4-(3-(2-fluorophenyl)ureido)-1H-pyrazol-1-yl)-N-(2-methylpyridin-4-yl)benzamide

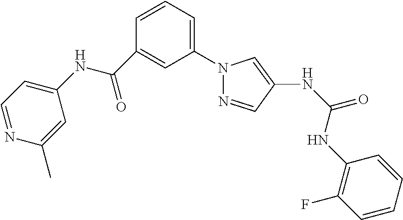 [SMILES: Cc1cc(NC(=O)c2cccc(-n3cc(NC(=O)Nc4ccccc4F)cn3)c2)ccn1]

Procedures in Scheme 1 were utilized to synthesize this compound. ¹H NMR (400 MHz, DMSO) δ 11.47 (s, 1H), 9.11 (s, 1H), 8.69 (s, 1H), 8.64 (d, J=6.9 Hz, 2H), 8.42 (s, 1H), 8.20-8.09 (m, 3H), 8.07 (d, J=6.5 Hz, 1H), 7.93-7.85 (m, 2H), 7.71 (t, J=8.0 Hz, 1H), 7.31-7.20 (m, 1H), 7.15 (t, J=7.7 Hz, 1H), 7.06-6.94 (m, 1H), 2.67 (s, 3H). LC-MS: 431 (M+H).

### Example I-51

### N-(2-methylpyridin-4-yl)-3-(4-(3-(o-tolyl)ureido)-1H-pyrazol-1-yl)benzamide

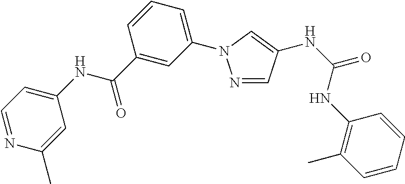 [SMILES: Cc1cc(NC(=O)c2cccc(-n3cc(NC(=O)Nc4ccccc4C)cn3)c2)ccn1]

Procedures in Scheme 1 were utilized to synthesize this compound. ¹H NMR (400 MHz, DMSO) δ 11.46 (s, 1H), 9.10 (s, 1H), 8.69-8.57 (m, 2H), 8.41 (s, 1H), 8.17-8.03 (m, 4H), 7.91-7.81 (m, 3H), 7.71 (t, J=8.0 Hz, 1H), 7.16 (dd, J=17.4, 7.7 Hz, 2H), 6.96 (t, J=6.9 Hz, 1H), 2.67 (s, 3H), 2.25 (s, 3H). LC-MS: 427 (M+H).

### Example I-52

### 3-(4-(3-(3-methoxyphenyl)ureido)-1H-pyrazol-1-yl)-N-(2-methylpyridin-4-yl)benzamide

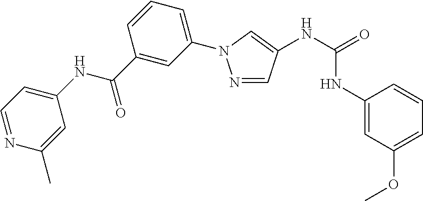 [SMILES: COc1cccc(NC(=O)Nc2cnn(-c3cccc(C(=O)Nc4ccnc(C)c4)c3)c2)c1]

Procedures in Scheme 1 were utilized to synthesize this compound. ¹H NMR (400 MHz, DMSO) δ 11.53 (s, 1H), 8.98 (s, 1H), 8.83 (s, 1H), 8.76-8.62 (m, 2H), 8.47 (t, J=1.9 Hz, 1H), 8.26-8.17 (m, 2H), 8.13 (d, J=6.7 Hz, 1H), 7.94 (d, J=8.3 Hz, 1H), 7.90 (s, 1H), 7.77 (t, J=8.0 Hz, 1H), 7.30 (t, J=2.2 Hz, 1H), 7.24 (t, J=8.1 Hz, 1H), 7.02 (dd, J=8.1, 1.1 Hz, 1H), 6.61 (dd, J=7.5, 2.5 Hz, 1H), 3.80 (s, 3H), 2.73 (s, 3H). LC-MS: 443 (M+H).

### Example I-53

### N-(2-methylpyridin-4-yl)-3-(4-(3-(m-tolyl)ureido)-1H-pyrazol-1-yl)benzamide

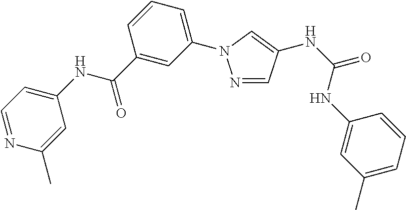 [SMILES: Cc1cccc(NC(=O)Nc2cnn(-c3cccc(C(=O)Nc4ccnc(C)c4)c3)c2)c1]

Procedures in Scheme 1 were utilized to synthesize this compound. LC-MS: 427 (M+H).

### Example I-54

### 3-(4-(3-(3-fluorophenyl)ureido)-1H-pyrazol-1-yl)-N-(2-methylpyridin-4-yl)benzamide

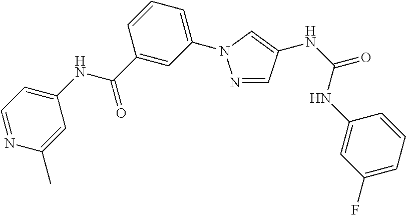 [SMILES: Cc1cc(NC(=O)c2cccc(-n3cc(NC(=O)Nc4cccc(F)c4)cn3)c2)ccn1]

Procedures in Scheme 1 were utilized to synthesize this compound. ¹H NMR (400 MHz, DMSO) δ 11.37 (s, 1H), 9.07 (s, 1H), 8.77 (s, 1H), 8.56 (d, J=5.7 Hz, 2H), 8.34 (s, 1H), 8.06 (d, J=5.9 Hz, 2H), 7.97 (s, 1H), 7.82 (d, J=8.0 Hz, 1H), 7.78 (s, 1H), 7.64 (t, J=8.0 Hz, 1H), 7.47 (dd, J=9.8, 2.2 Hz, 1H), 7.24 (dd, J=15.2, 8.2 Hz, 1H), 7.07 (d, J=9.3 Hz, 1H), 6.72 (d, J=2.3 Hz, 1H), 2.60 (s, 3H). LC-MS: 431 (M+H).

### Example I-55

### 3-(4-(3-(4-chlorophenyl)ureido)-1H-pyrazol-1-yl)-N-(2-methylpyridin-4-yl)benzamide

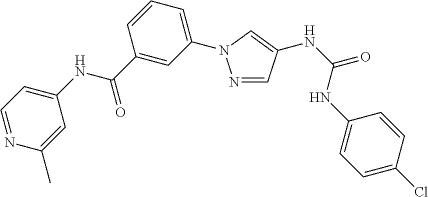 [SMILES: Cc1cc(NC(=O)c2cccc(-n3cc(NC(=O)Nc4ccc(Cl)cc4)cn3)c2)ccn1]

Procedures in Scheme 1 were utilized to synthesize this compound. LC-MS: 447 (M+H).

### Example I-56

### N-(2-methylpyridin-4-yl)-3-(4-(3-(p-tolyl)ureido)-1H-pyrazol-1-yl)benzamide

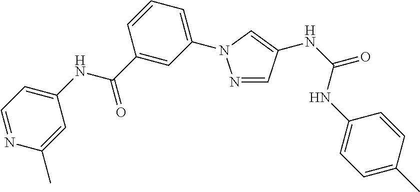 [SMILES: Cc1ccc(NC(=O)Nc2cnn(-c3cccc(C(=O)Nc4ccnc(C)c4)c3)c2)cc1]

Procedures in Scheme 1 were utilized to synthesize this compound. LC-MS: 427 (M+H).

### Example I-57

### N-(1-(3-((2-methylpyridin-4-yl)carbamoyl)phenyl)-1H-pyrazol-4-yl)-1H-1,2,4-triazole-3-carboxamide

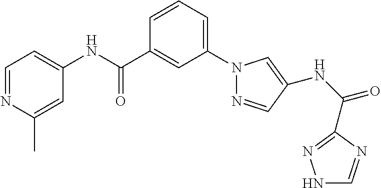 [SMILES: Cc1cc(NC(=O)c2cccc(-n3cc(NC(=O)c4nc[nH]n4)cn3)c2)ccn1]

Procedures in Scheme 3 were utilized to synthesize this compound. LC-MS: 389 (M+H).

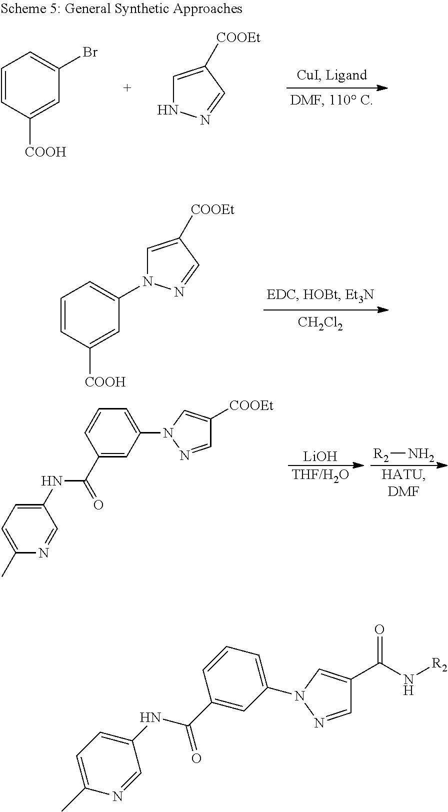

A mixture of ethyl 1H-pyrazole-4-carboxylate (10 mmol), 3-bromobenzoic acid (20 mmol), CuI (2.0 mmol), trans-N,N-dimethylcyclohexane-1,2-diamine (4.0 mmol) and Cs2CO3 (30 mmol) in DMF (20 mL) was purged with argon and stirred for 12 h at 100° C. in a sealed tube. The reaction mixture was cooled, and filtered through a pad of silica gel and rinsed with EtOAc. The resulting solution was concentrated in vacuo to yield a crude residue which was purified by chromatography on silica gel (EtOAc/hexane) to provide 3-(4-(ethoxycarbonyl)-1H-pyrazol-1-yl)benzoic acid (75% yield).

A solution of 3-(4-(ethoxycarbonyl)-1H-pyrazol-1-yl)benzoic acid (5.0 mmol) in CH₂Cl₂ (20 mL) was added EDC (10 mmol), HOBt (10 mmol) and diisopropyl ethyl amine (15 mmol) and stirred for 30 min. Then the 6-methylpyridin-3-amine (5.5 mmol) was added and the resulting mixture was stirred over night. Water (50 ml) was added to the reaction mixture and extracted with EtOAc (2×100 mL). The resulting solution was concentrated in vacuo to yield a crude product.

A mixture of ethyl 1-(3-((6-methylpyridin-3-yl)carbamoyl)phenyl)-1H-pyrazole-4-carboxylate (5 mmol) and LiOH (2.0 g) was stirred in THF (30 mL)/water (30 mL) at RT for 2-3 hours. The resulting homogenous reaction was acidified with 2M HCl (20 mL) and extracted with EtOAc (2×100 mL). The combined organic extracts were washed with water (100 mL), dried (Na₂SO4) and evaporated. The crude residue was dried under vacuum and used without further purification.

The 1-(3-((6-methylpyridin-3-yl)carbamoyl)phenyl)-1H-pyrazole-4-carboxylic acid (0.1 mmol) was added HATU (2 eq.) and diisopropyl ethyl amine (2 eq.) in DMF, stirred for 30 min. The amide (0.12 mmol) was added to the mixture and the resulting mixture was stirred over night. The residue was purified by reverse-phase preparative HPLC to give the product.

### Example I-58

### N-(2-chlorobenzyl)-1-(3-((6-methylpyridin-3-yl)carbamoyl)phenyl)-1H-pyrazole-4-carboxamide

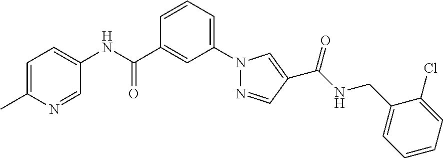 [SMILES: Cc1ccc(NC(=O)c2cccc(-n3cc(C(=O)NCc4ccccc4Cl)cn3)c2)cn1]

Procedures in Scheme 5 were utilized to synthesize this compound. ¹H NMR (400 MHz, DMSO) δ 9.12 (s, 1H), 9.08 (s, 1H), 8.88 (d, J=3.8 Hz, 1H), 8.83 (t, J=5.8 Hz, 1H), 8.48 (t, J=1.8 Hz, 1H), 8.42 (d, J=9.2 Hz, 1H), 8.30 (s, 1H), 8.15 (dd, J=8.1, 1.4 Hz, 1H), 7.94 (d, J=20.5 Hz, 1H), 7.74 (dd, J=15.3, 7.3 Hz, 2H), 7.48 (dd, J=7.5, 1.7 Hz, 1H), 7.42 (dd, J=7.4, 1.9 Hz, 1H), 7.38-7.27 (m, 2H), 4.55 (d, J=5.7 Hz, 2H), 2.61 (s, 3H). LC-MS: 446 (M+H).

### Example I-59

### 3-(4-(2-(2-chlorophenyl)acetamido)-1H-pyrazol-1-yl)-N-(6-methylpyridin-3-yl)benzamide

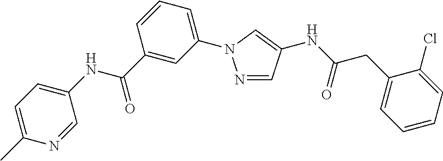 [SMILES: Cc1ccc(NC(=O)c2cccc(-n3cc(NC(=O)Cc4ccccc4Cl)cn3)c2)cn1]

Procedures in Scheme 3 were utilized to synthesize this compound. LC-MS: 446 (M+H).

### Example I-60

### N-(2-fluorobenzyl)-1-(3-((6-methylpyridin-3-yl)carbamoyl)phenyl)-1H-pyrazole-4-carboxamide

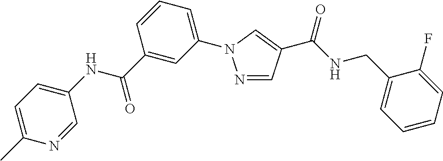 [SMILES: Cc1ccc(NC(=O)c2cccc(-n3cc(C(=O)NCc4ccccc4F)cn3)c2)cn1]

Procedures in Scheme 5 were utilized to synthesize this compound. LC-MS: 430 (M+H).

### Example I-61

### N-(3-methylbenzyl)-1-(3-((6-methylpyridin-3-yl)carbamoyl)phenyl)-1H-pyrazole-4-carboxamide

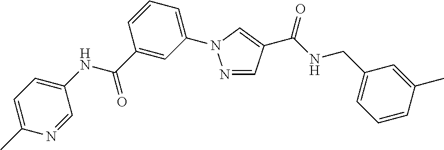

Procedures in Scheme 5 were utilized to synthesize this compound. LC-MS: 426 (M+H).

### Example I-70

### 3-(4-((1H-benzo[d]imidazol-2-yl)amino)-1H-pyrazol-1-yl)-N-(6-methylpyridin-3-yl)benzamide

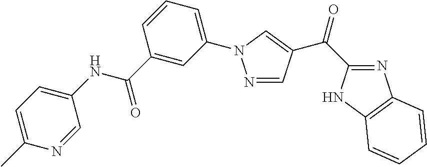 [SMILES: Cc1ccc(NC(=O)c2cccc(-n3cc(C(=O)c4nc5ccccc5[nH]4)cn3)c2)cn1]

Procedures in Scheme 4 were utilized to synthesize this compound. LC-MS: 410 (M+H).

### Example I-71

### 3-(4-((6-fluorobenzo[d]thiazol-2-yl)amino)-1H-pyrazol-1-yl)-N-(6-methylpyridin-3-yl)benzamide

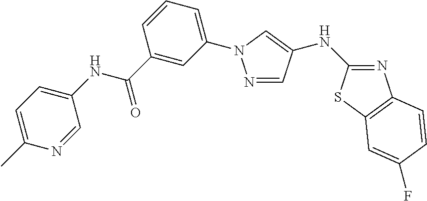 [SMILES: Cc1ccc(NC(=O)c2cccc(-n3cc(Nc4nc5ccc(F)cc5s4)cn3)c2)cn1]

Procedures in Scheme 4 were utilized to synthesize this compound. LC-MS: 445 (M+H).

### Example I-72

### 3-(4-(benzo[d]thiazol-2-ylamino)-1H-pyrazol-1-yl)-N-(6-methylpyridin-3-yl)benzamide

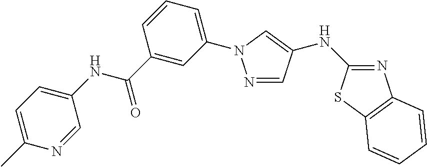 [SMILES: Cc1ccc(NC(=O)c2cccc(-n3cc(Nc4nc5ccccc5s4)cn3)c2)cn1]

Procedures in Scheme 4 were utilized to synthesize this compound. LC-MS: 427 (M+H).

### Example I-73

### N-(1-(3-((2-methylpyridin-4-yl)carbamoyl)phenyl)-1H-pyrazol-4-yl)-1H-indole-2-carboxamide

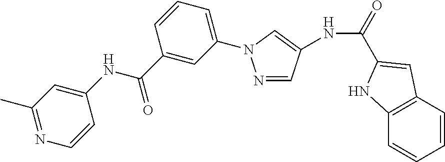 [SMILES: Cc1cc(NC(=O)c2cccc(-n3cc(NC(=O)c4cc5ccccc5[nH]4)cn3)c2)ccn1]

Procedures in Scheme 3 were utilized to synthesize this compound. LC-MS: 437 (M+H).

### Example I-74

### 7-chloro-N-(1-(3-((2-methylpyridin-4-yl)carbamoyl)phenyl)-1H-pyrazol-4-yl)-1H-indole-2-carboxamide

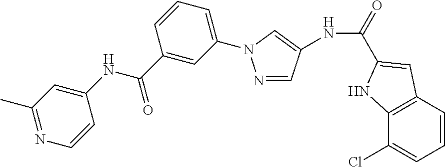 [SMILES: Cc1cc(NC(=O)c2cccc(-n3cc(NC(=O)c4cc5cccc(Cl)c5[nH]4)cn3)c2)ccn1]

Procedures in Scheme 3 were utilized to synthesize this compound. LC-MS: 471 (M+H).

### Example I-75

### 5-chloro-N-(1-(3-((2-methylpyridin-4-yl)carbamoyl)phenyl)-1H-pyrazol-4-yl)-1 H-indole-2-carboxamide

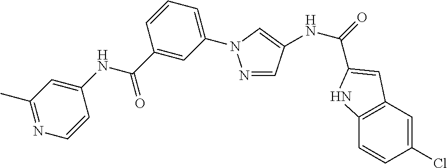 [SMILES: Cc1cc(NC(=O)c2cccc(-n3cc(NC(=O)c4cc5cc(Cl)ccc5[nH]4)cn3)c2)ccn1]

Procedures in Scheme 3 were utilized to synthesize this compound. LC-MS: 471 (M+H).

### Example I-76

### 6-fluoro-N-(1-(3-((6-methylpyridin-3-yl)carbamoyl)phenyl)-1H-pyrazol-4-yl)-1H-benzo[d]imidazole-2-carboxamide

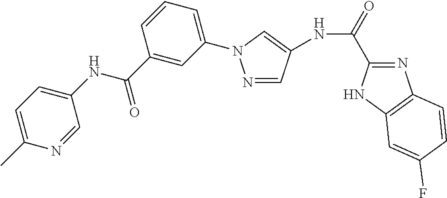 [SMILES: Cc1ccc(NC(=O)c2cccc(-n3cc(NC(=O)c4nc5ccc(F)cc5[nH]4)cn3)c2)cn1]

Procedures in Scheme 3 were utilized to synthesize this compound. LC-MS: 456 (M+H).

### Example I-77

### 5-bromo-N-(1-(3-((6-methylpyridin-3-yl)carbamoyl)phenyl)-1H-pyrazol-4-yl)-1 H-benzo[d]imidazole-2-carboxamide

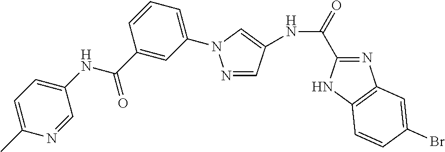 [SMILES: Cc1ccc(NC(=O)c2cccc(-n3cc(NC(=O)c4nc5cc(Br)ccc5[nH]4)cn3)c2)cn1]

Procedures in Scheme 3 were utilized to synthesize this compound. LC-MS: 516 (M+H).

### Example I-78

### N-(1-(3-((6-methylpyridin-3-yl)carbamoyl)phenyl)-1H-pyrazol-4-yl)-1H-indole-3-carboxamide

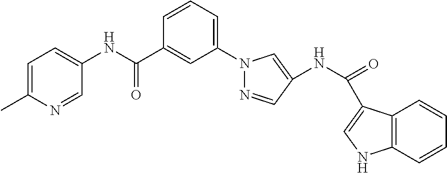 [SMILES: Cc1ccc(NC(=O)c2cccc(-n3cc(NC(=O)c4c[nH]c5ccccc45)cn3)c2)cn1]

Procedures in Scheme 3 were utilized to synthesize this compound. LC-MS: 437 (M+H).

### Example I-83

### N-(1-(3-((6-methylpyridin-3-yl)carbamoyl)phenyl)-1H-pyrazol-4-yl)-1,2,3,4-tetrahydroisoquinoline-3-carboxamide

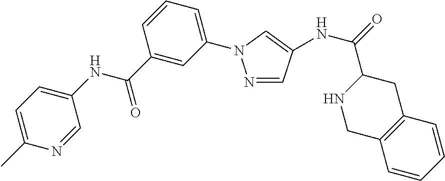 [SMILES: Cc1ccc(NC(=O)c2cccc(-n3cc(NC(=O)C4Cc5ccccc5CN4)cn3)c2)cn1]

Procedures in Scheme 3 were utilized to synthesize this compound. LC-MS: 453 (M+H).

### Example I-84

### 3-(4-(3-(4-fluorophenyl)ureido)-1H-pyrazol-1-yl)-N-(6-methylpyridin-3-yl)benzamide

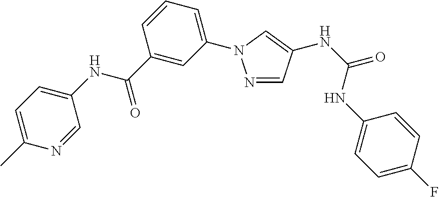 [SMILES: Cc1ccc(NC(=O)c2cccc(-n3cc(NC(=O)Nc4ccc(F)cc4)cn3)c2)cn1]

Procedures in Scheme 1 were utilized to synthesize this compound. LC-MS: 431 (M+H).

### Example I-85

### 3-(4-(3-(2-chlorophenyl)ureido)-1H-pyrazol-1-yl)-N-(1-methyl-1H-pyrazol-4-yl)benzamide

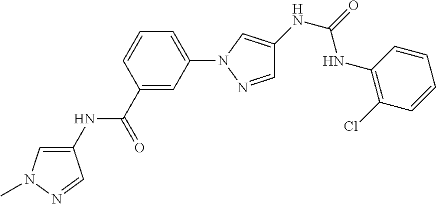 [SMILES: Cn1cc(NC(=O)c2cccc(-n3cc(NC(=O)Nc4ccccc4Cl)cn3)c2)cn1]

Procedures in Scheme 1 were utilized to synthesize this compound. ¹H NMR (400 MHz, DMSO) δ 10.61 (s, 1H), 9.43 (s, 1H), 8.60 (s, 1H), 8.39 (s, 1H), 8.35 (t, J=1.8 Hz, 1H), 8.21 (dd, J=8.3, 1.5 Hz, 1H), 8.07 (s, 1H), 8.04-7.97 (m, 1H), 7.90-7.81 (m, 2H), 7.63 (dd, J=13.0, 4.9 Hz, 2H), 7.47 (dd, J=8.0, 1.5 Hz, 1H), 7.34-7.26 (m, 1H), 7.11-6.99 (m, 1H), 3.35 (d, J=10.3 Hz, 3H). LC-MS: 436 (M+H).

### Example I-86

### 3-(4-(3-(2-chlorophenyl)ureido)-1H-pyrazol-1-yl)-N-(1-isopropyl-1H-pyrazol-4-yl)benzamide

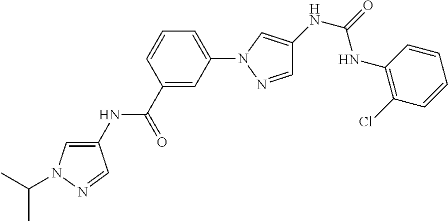 [SMILES: CC(C)n1cc(NC(=O)c2cccc(-n3cc(NC(=O)Nc4ccccc4Cl)cn3)c2)cn1]

Procedures in Scheme 1 were utilized to synthesize this compound. LC-MS: 464 (M+H).

### Example I-87

### 3-(4-(3-(2-chlorophenyl)ureido)-1H-pyrazol-1-yl)-N-(1-methyl-1H-pyrazol-4-yl)benzamide

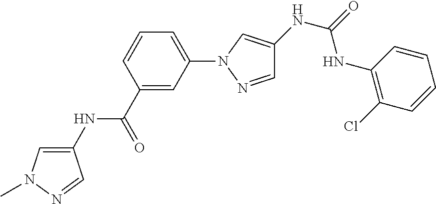 [SMILES: Cn1cc(NC(=O)c2cccc(-n3cc(NC(=O)Nc4ccccc4Cl)cn3)c2)cn1]

Procedures in Scheme 1 were utilized to synthesize this compound. LC-MS: 436 (M+H).

### Example I-88

### 3-(4-(3-(2-chlorophenyl)ureido)-1H-pyrazol-1-yl)-N-(1-methyl-1H-1,2,4-triazol-3-yl)benzamide

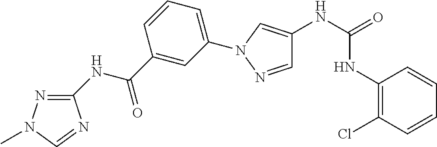 [SMILES: Cn1cnc(NC(=O)c2cccc(-n3cc(NC(=O)Nc4ccccc4Cl)cn3)c2)n1]

Procedures in Scheme 1 were utilized to synthesize this compound. LC-MS: 437 (M+H).

### Example I-89

### 3-(4-(3-(2-chlorophenyl)ureido)-1H-pyrazol-1-yl)-N-(3-cyclopropyl-1-methyl-1 H-pyrazol-5-yl)benzamide

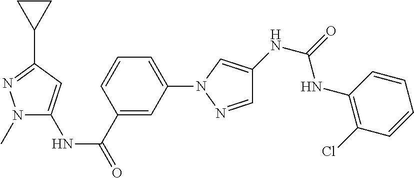 [SMILES: Cn1nc(C2CC2)cc1NC(=O)c1cccc(-n2cc(NC(=O)Nc3ccccc3Cl)cn2)c1]

Procedures in Scheme 1 were utilized to synthesize this compound. ¹H NMR (400 MHz, DMSO) δ 10.44 (s, 1H), 8.61 (s, 1H), 8.37 (d, J=7.2 Hz, 2H), 8.20 (dd, J=8.3, 1.5 Hz, 1H), 8.07 (d, J=9.4 Hz, 1H), 7.85 (d, J=5.4 Hz, 2H), 7.65 (t, J=7.9 Hz, 1H), 7.47 (dd, J=8.0, 1.5 Hz, 1H), 7.39-7.24 (m, 1H), 7.03 (dd, J=10.6, 4.7 Hz, 1H), 5.98 (s, 1H), 3.62 (s, 3H), 1.92-1.78 (m, 1H), 0.89-0.79 (m, 2H), 0.69-0.57 (m, 2H). LC-MS: 476 (M+H).

### Example I-90

### 3-(4-(3-(2-chlorophenyl)ureido)-1H-pyrazol-1-yl)-N-(4,5,6,7-tetrahydrobenzo[d]thiazol-2-yl)benzamide

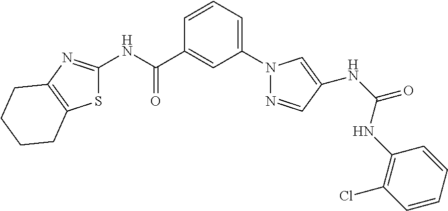 [SMILES: O=C(Nc1cnn(-c2cccc(C(=O)Nc3nc4c(s3)CCCC4)c2)c1)Nc1ccccc1Cl]

Procedures in Scheme 1 were utilized to synthesize this compound. LC-MS: 493 (M+H).

### Example I-91

### 3-(4-(3-(2-chlorophenyl)ureido)-1H-pyrazol-1-yl)-N-(4-(pyridin-4-yl)thiazol-2-yl)benzamide

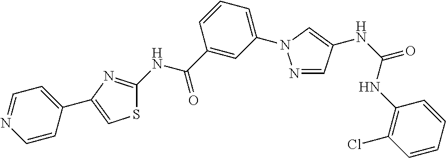 [SMILES: O=C(Nc1cnn(-c2cccc(C(=O)Nc3nc(-c4ccncc4)cs3)c2)c1)Nc1ccccc1Cl]

Procedures in Scheme 1 were utilized to synthesize this compound. LC-MS: 516 (M+H).

### Example I-92

### 3-(4-(3-(2-chlorophenyl)ureido)-1H-pyrazol-1-yl)-N-(1-(piperidin-4-yl)-1H-pyrazol-4-yl)benzamide

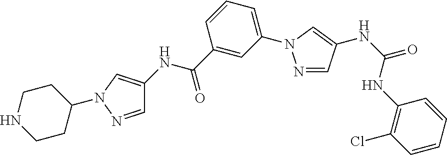 [SMILES: O=C(Nc1cnn(-c2cccc(C(=O)Nc3cnn(C4CCNCC4)c3)c2)c1)Nc1ccccc1Cl]

Procedures in Scheme 1 were utilized to synthesize this compound. ¹H NMR (400 MHz, MeOD) δ 8.50 (s, 1H), 8.33 (s, 1H), 8.25 (s, 1H), 8.15 (dd, J=8.3, 1.5 Hz, 1H), 7.99 (d, J=5.9 Hz, 1H), 7.87 (d, J=8.4 Hz, 1H), 7.76 (s, 1H), 7.73 (s, 1H), 7.66 (t, J=7.9 Hz, 1H), 7.46-7.39 (m, 1H), 7.30 (d, J=6.9 Hz, 1H), 7.11-7.01 (m, 1H), 3.59 (d, J=13.9 Hz, 2H), 3.51-3.46 (m, 1H), 3.29-3.19 (m, 2H), 2.40-2.27 (m, 4H). LC-MS: 505 (M+H).

### Example I-93

### 3-(4-(3-(2-chlorophenyl)ureido)-1H-pyrazol-1-yl)-N-(1,3-dimethyl-1H-pyrazol-4-yl)benzamide

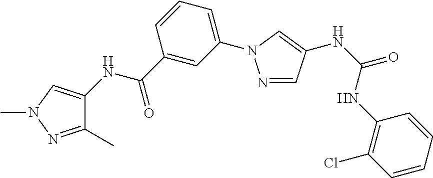 [SMILES: Cc1nn(C)cc1NC(=O)c1cccc(-n2cc(NC(=O)Nc3ccccc3Cl)cn2)c1]

Procedures in Scheme 1 were utilized to synthesize this compound. ¹H NMR (400 MHz, DMSO) δ 9.88 (s, 1H), 9.42 (s, 1H), 8.59 (s, 1H), 8.38 (s, 1H), 8.32 (s, 1H), 8.21 (dd, J=8.3, 1.5 Hz, 1H), 8.01 (d, J=9.5 Hz, 1H), 7.90 (s, 1H), 7.86-7.78 (m, 2H), 7.62 (t, J=7.9 Hz, 1H), 7.47 (dd, J=8.0, 1.4 Hz, 1H), 7.31 (t, J=7.1 Hz, 1H), 7.03 (dd, J=10.9, 4.5 Hz, 1H), 3.77 (s, 3H), 2.17 (s, 3H). LC-MS: 450 (M+H).

### Example I-94

### 3-(4-(3-(2-chlorophenyl)ureido)-1H-pyrazol-1-yl)-N-(1,3-dimethyl-1H-pyrazol-4-yl)benzamide

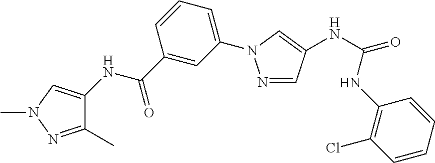 [SMILES: Cc1nn(C)cc1NC(=O)c1cccc(-n2cc(NC(=O)Nc3ccccc3Cl)cn2)c1]

Procedures in Scheme 1 were utilized to synthesize this compound. ¹H NMR (400 MHz, MeOD) δ 8.49 (s, 1H), 8.35-8.30 (m, 1H), 8.15 (dd, J=8.3, 1.5 Hz, 1H), 8.00-7.91 (m, 2H), 7.87 (d, J=8.3 Hz, 1H), 7.77 (s, 1H), 7.65 (t, J=8.0 Hz, 1H), 7.44 (dd, J=8.0, 1.4 Hz, 1H), 7.36-7.21 (m, 2H), 7.12-6.98 (m, 1H), 2.30 (s, 3H), 1.52 (d, J=6.7 Hz, 6H). LC-MS: 450 (M+H).

### Example I-95

### 3-(4-(3-(2-chlorophenyl)ureido)-1H-pyrazol-1-yl)-N-(1-(2-hydroxyethyl)-3-methyl-1H-pyrazol-4-yl)benzamide

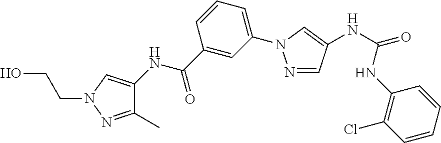 [SMILES: Cc1nn(CCO)cc1NC(=O)c1cccc(-n2cc(NC(=O)Nc3ccccc3Cl)cn2)c1]

Procedures in Scheme 1 were utilized to synthesize this compound. LC-MS: 480 (M+H).

### Example I-96

### Methyl-2-(3-(4-(3-(2-chlorophenyl)ureido)-1H-pyrazol-1-yl)benzamido)-4-methylthiazole-5-carboxylate

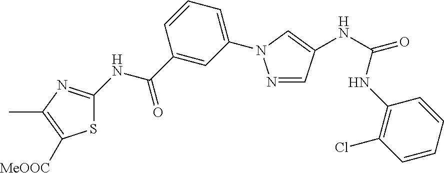 [SMILES: COC(=O)c1sc(NC(=O)c2cccc(-n3cc(NC(=O)Nc4ccccc4Cl)cn3)c2)nc1C]

Procedures in Scheme 1 were utilized to synthesize this compound. LC-MS: 511 (M+H).

### Example I-97

### 3-(4-(3-(2-chlorophenyl)ureido)-1H-pyrazol-1-yl)-N-(1-methyl-1H-pyrazol-5-yl)benzamide

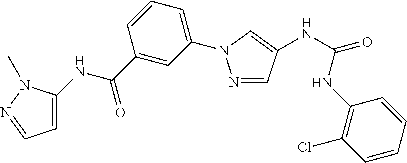 [SMILES: Cn1nccc1NC(=O)c1cccc(-n2cc(NC(=O)Nc3ccccc3Cl)cn2)c1]

Procedures in Scheme 1 were utilized to synthesize this compound. LC-MS: 436 (M+H).

### Example I-98

### 3-(4-(3-(2-chlorophenyl)ureido)-1H-pyrazol-1-yl)-N-(1-methyl-1H-pyrazol-3-yl)benzamide

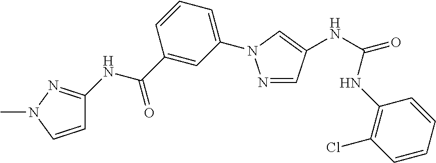 [SMILES: Cn1ccc(NC(=O)c2cccc(-n3cc(NC(=O)Nc4ccccc4Cl)cn3)c2)n1]

Procedures in Scheme 1 were utilized to synthesize this compound. ¹H NMR (400 MHz, DMSO) δ 11.07 (s, 1H), 9.42 (s, 1H), 8.66 (s, 1H), 8.38 (s, 2H), 8.23 (dd, J=8.3, 1.5 Hz, 1H), 8.02 (dd, J=8.1, 1.3 Hz, 1H), 7.88 (d, J=8.0 Hz, 1H), 7.82 (s, 1H), 7.67-7.57 (m, 2H), 7.47 (dd, J=8.0, 1.5 Hz, 1H), 7.38-7.26 (m, 1H), 7.08-6.97 (m, 1H), 6.63 (d, J=2.2 Hz, 1H), 3.81 (s, 3H). LC-MS: 436 (M+H).

### Example I-99

### 3-(4-(3-(2-chlorophenyl)ureido)-1H-pyrazol-1-yl)-N-(1-methyl-3-phenyl-1H-pyrazol-5-yl)benzamide

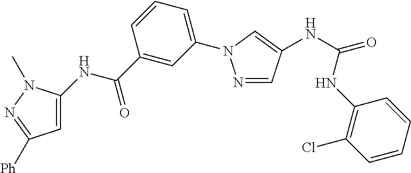 [SMILES: Cn1nc(-c2ccccc2)cc1NC(=O)c1cccc(-n2cc(NC(=O)Nc3ccccc3Cl)cn2)c1]

Procedures in Scheme 1 were utilized to synthesize this compound. LC-MS: 512 (M+H).

### Example I-100

### 3-(4-(3-(2-chlorophenyl)ureido)-1H-pyrazol-1-yl)-N-(1-(2-(dimethylamino)ethyl)-1H-pyrazol-4-yl)benzamide

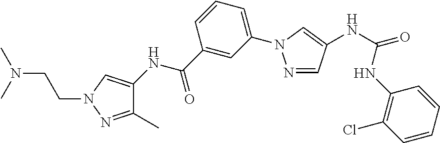 [SMILES: Cc1nn(CCN(C)C)cc1NC(=O)c1cccc(-n2cc(NC(=O)Nc3ccccc3Cl)cn2)c1]

Procedures in Scheme 1 were utilized to synthesize this compound. ¹H NMR (400 MHz, MeOD) δ 8.36 (s, 1H), 8.20 (t, J=1.8 Hz, 1H), 8.13 (s, 1H), 8.02 (dd, J=8.3, 1.5 Hz, 1H), 7.87-7.82 (m, 1H), 7.74 (d, J=8.2 Hz, 1H), 7.68 (s, 1H), 7.64 (s, 1H), 7.52 (t, J=8.0 Hz, 1H), 7.31 (dd, J=8.0, 1.4 Hz, 1H), 7.18 (d, J=1.3 Hz, 1H), 6.95 (dd, J=7.7, 1.3 Hz, 1H), 4.57-4.32 (m, 2H), 3.59 (t, J=5.7 Hz, 2H), 2.87 (s, 6H). LC-MS: 493 (M+H).

### Example I-101

### 3-(4-(3-(2-chlorophenyl)ureido)-1H-pyrazol-1-yl)-N-(1-(2-(pyrrolidin-1-yl)ethyl)-1H-pyrazol-4-yl)benzamide

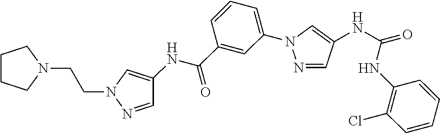 [SMILES: O=C(Nc1cnn(-c2cccc(C(=O)Nc3cnn(CCN4CCCC4)c3)c2)c1)Nc1ccccc1Cl]

Procedures in Scheme 1 were utilized to synthesize this compound. LC-MS: 519 (M+H).

### Example I-102

### N-(3-(tert-butyl)-1-methyl-1H-pyrazol-5-yl)-3-(4-(3-(2-chlorophenyl)ureido)-1H-pyrazol-1-yl)benzamide

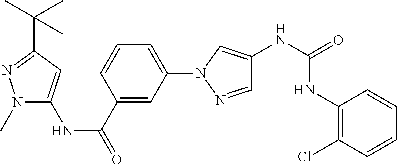 [SMILES: Cn1nc(C(C)(C)C)cc1NC(=O)c1cccc(-n2cc(NC(=O)Nc3ccccc3Cl)cn2)c1]

Procedures in Scheme 1 were utilized to synthesize this compound. LC-MS: 492 (M+H).

### Example I-103

### 3-(4-(3-(2-chlorophenyl)ureido)-1H-pyrazol-1-yl)-N-(1-methyl-3-(thiophen-2-yl)-1H-pyrazol-5-yl)benzamide

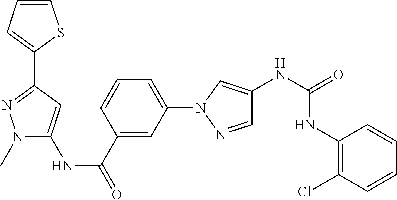 [SMILES: Cn1nc(-c2cccs2)cc1NC(=O)c1cccc(-n2cc(NC(=O)Nc3ccccc3Cl)cn2)c1]

Procedures in Scheme 1 were utilized to synthesize this compound. LC-MS: 518 (M+H).

### Example I-104

### 3-(4-(3-(2-chlorophenyl)ureido)-1H-pyrazol-1-yl)-N-(3-methyl-1-(2-(pyrrolidin-1-yl)ethyl)-1H-pyrazol-4-yl)benzamide

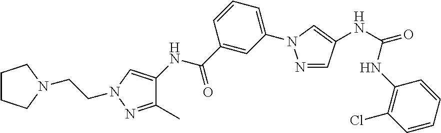 [SMILES: Cc1nn(CCN2CCCC2)cc1NC(=O)c1cccc(-n2cc(NC(=O)Nc3ccccc3Cl)cn2)c1]

Procedures in Scheme 1 were utilized to synthesize this compound. LC-MS: 533 (M+H).

### Example I-105

### 3-(4-(3-(2-chlorophenyl)ureido)-1H-pyrazol-1-yl)-N-(5-methyl-1-(2-(pyrrolidin-1-yl)ethyl)-1H-pyrazol-4-yl)benzamide

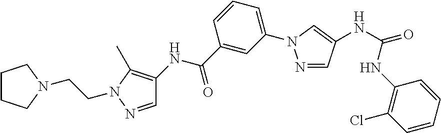 [SMILES: Cc1c(NC(=O)c2cccc(-n3cc(NC(=O)Nc4ccccc4Cl)cn3)c2)cnn1CCN1CCCC1]

Procedures in Scheme 1 were utilized to synthesize this compound. LC-MS: 533 (M+H).

### Example I-106

### 3-(4-(3-(2-chlorophenyl)ureido)-1H-pyrazol-1-yl)-N-(5-methyl-1-(2-(piperidin-1-yl)ethyl)-1H-pyrazol-4-yl)benzamide

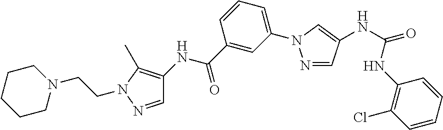 [SMILES: Cc1c(NC(=O)c2cccc(-n3cc(NC(=O)Nc4ccccc4Cl)cn3)c2)cnn1CCN1CCCCC1]

Procedures in Scheme 1 were utilized to synthesize this compound. LC-MS: 547 (M+H).

### Example I-107

### 3-(4-(3-(2-chlorophenyl)ureido)-1H-pyrazol-1-yl)-N-(3-methyl-1-(2-(piperidin-1-yl)ethyl)-1H-pyrazol-4-yl)benzamide

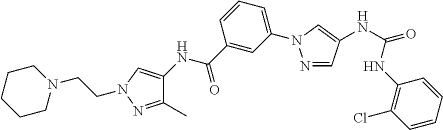 [SMILES: Cc1nn(CCN2CCCCC2)cc1NC(=O)c1cccc(-n2cc(NC(=O)Nc3ccccc3Cl)cn2)c1]

Procedures in Scheme 1 were utilized to synthesize this compound. LC-MS: 547 (M+H).

### Example I-108

### 3-(4-(3-(2-chlorophenyl)ureido)-1H-pyrazol-1-yl)-N-(3,5-dimethyl-1-(2-(piperidin-1-yl)ethyl)-1H-pyrazol-4-yl)benzamide

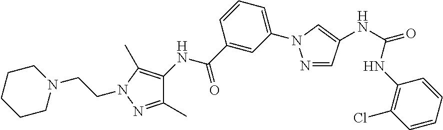 [SMILES: Cc1nn(CCN2CCCCC2)c(C)c1NC(=O)c1cccc(-n2cc(NC(=O)Nc3ccccc3Cl)cn2)c1]

Procedures in Scheme 1 were utilized to synthesize this compound. ¹H NMR (400 MHz, DMSO) δ 9.79 (s, 1H), 9.44 (s, 1H), 8.61 (s, 1H), 8.38 (d, J=12.9 Hz, 2H), 8.19 (dd, J=8.3, 1.5 Hz, 1H), 8.04 (dd, J=7.7, 1.8 Hz, 1H), 7.89-7.81 (m, 2H), 7.64 (t, J=7.9 Hz, 1H), 7.47 (dd, J=8.0, 1.5 Hz, 1H), 7.34-7.22 (m, 1H), 7.10-6.97 (m, 1H), 4.38 (d, J=6.8 Hz, 2H), 3.50 (s, 4H), 2.96 (s, 2H), 2.19 (s, 3H), 2.07 (s, 3H), 1.84 (s, 2H), 1.69 (s, 4H). LC-MS: 561 (M+H).

### Example I-109

### 3-(4-(3-(2-fluorophenyl)ureido)-1H-pyrazol-1-yl)-N-(5-methyl-1-(2-(pyrrolidin-1-yl)ethyl)-1H-pyrazol-4-yl)benzamide

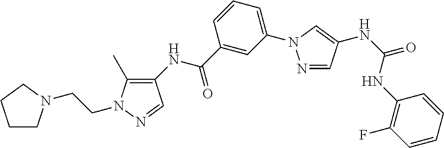 [SMILES: Cc1c(NC(=O)c2cccc(-n3cc(NC(=O)Nc4ccccc4F)cn3)c2)cnn1CCN1CCCC1]

Procedures in Scheme 1 were utilized to synthesize this compound. LC-MS: 517 (M+H).

### Example I-110

### 3-(4-(3-(2-fluorophenyl)ureido)-1H-pyrazol-1-yl)-N-(3-methyl-1-(2-(pyrrolidin-1-yl)ethyl)-1H-pyrazol-4-yl)benzamide

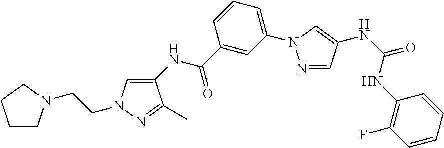 [SMILES: Cc1nn(CCN2CCCC2)cc1NC(=O)c1cccc(-n2cc(NC(=O)Nc3ccccc3F)cn2)c1]

Procedures in Scheme 1 were utilized to synthesize this compound. ¹H NMR (400 MHz, DMSO) δ 9.99 (s, 1H), 9.05 (s, 1H), 8.65 (d, J=2.3 Hz, 1H), 8.59 (s, 1H), 8.31 (s, 1H), 8.21-8.11 (m, 2H), 8.02 (dd, J=8.1, 1.3 Hz, 1H), 7.88-7.78 (m, 2H), 7.63 (t, J=7.9 Hz, 1H), 7.25 (ddd, J=11.6, 8.2, 1.4 Hz, 1H), 7.15 (t, J=7.3 Hz, 1H), 7.07-6.96 (m, 1H), 4.41 (s, 2H), 3.61 (s, 2H), 3.52-3.40 (m, 2H), 3.02 (s, 2H), 2.24 (s, 3H), 1.96 (d, J=39.9 Hz, 4H). LC-MS: 517 (M+H).

### Example I-111

### N-(5-methyl-1-(2-(pyrrolidin-1-yl)ethyl)-1H-pyrazol-4-yl)-3-(4-(3-(m-tolyl)ureido)-1 H-pyrazol-1-yl)benzamide

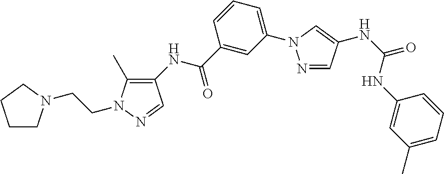 [SMILES: Cc1cccc(NC(=O)Nc2cnn(-c3cccc(C(=O)Nc4cnn(CCN5CCCC5)c4C)c3)c2)c1]

Procedures in Scheme 1 were utilized to synthesize this compound. LC-MS: 513 (M+H).

### Example I-112

### N-(3-methyl-1-(2-(pyrrolidin-1-yl)ethyl)-1H-pyrazol-4-yl)-3-(4-(3-(m-tolyl)ureido)-1 H-pyrazol-1-yl)benzamide

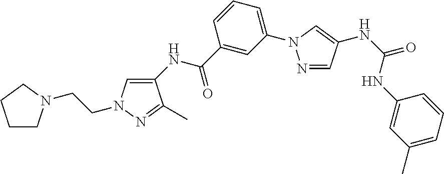 [SMILES: Cc1cccc(NC(=O)Nc2cnn(-c3cccc(C(=O)Nc4cn(CCN5CCCC5)nc4C)c3)c2)c1]

Procedures in Scheme 1 were utilized to synthesize this compound. ¹H NMR (400 MHz, MeOD) δ 8.46 (s, 1H), 8.32 (t, J=1.9 Hz, 1H), 8.03 (s, 1H), 7.98 (ddd, J=8.1, 2.3, 0.9 Hz, 1H), 7.92-7.83 (m, 1H), 7.76 (s, 1H), 7.65 (t, J=8.0 Hz, 1H), 7.32-7.13 (m, 3H), 6.88 (d, J=7.3 Hz, 1H), 4.51 (t, J=5.7 Hz, 2H), 3.79-3.66 (m, 4H), 3.25-3.11 (m, 2H), 2.34 (s, 3H), 2.31 (s, 3H), 2.20 (s, 2H), 2.05 (s, 2H). LC-MS: 513 (M+H).

### Example I-113

### 3-(4-(3-(3-methoxyphenyl)ureido)-1H-pyrazol-1-yl)-N-(5-methyl-1-(2-(pyrrolidin-1-yl)ethyl)-1H-pyrazol-4-yl)benzamide

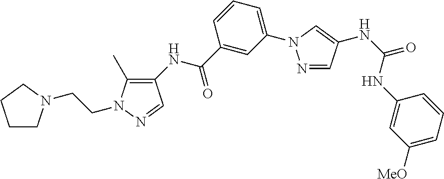 [SMILES: COc1cccc(NC(=O)Nc2cnn(-c3cccc(C(=O)Nc4cnn(CCN5CCCC5)c4C)c3)c2)c1]

Procedures in Scheme 1 were utilized to synthesize this compound. ¹H NMR (400 MHz, MeOD) δ 8.44 (s, 1H), 8.32 (s, 1H), 8.02 (s, OH), 7.99-7.90 (m, 1H), 7.91-7.81 (m, 1H), 7.77 (s, 1H), 7.68 (s, 1H), 7.63 (s, 1H), 7.25-7.08 (m, 2H), 6.95 (ddd, J=8.0, 1.9, 0.8 Hz, 1H), 6.61 (dd, J=8.2, 2.5 Hz, 1H), 4.50 (d, J=5.9 Hz, 2H), 3.80 (s, 3H), 3.73 (t, J=5.8 Hz, 4H), 3.16 (s, 2H), 2.31 (s, 3H), 2.17 (d, J=4.4 Hz, 2H), 2.06 (s, 2H). LC-MS: 529 (M+H).

### Example I-114

### 3-(4-(3-(3-methoxyphenyl)ureido)-1H-pyrazol-1-yl)-N-(3-methyl-1-(2-(pyrrolidin-1-yl)ethyl)-1H-pyrazol-4-yl)benzamide

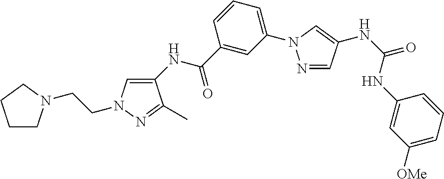 [SMILES: COc1cccc(NC(=O)Nc2cnn(-c3cccc(C(=O)Nc4cn(CCN5CCCC5)nc4C)c3)c2)c1]

Procedures in Scheme 1 were utilized to synthesize this compound. LC-MS: 529 (M+H).

### Example I-115

### 3-(4-(3-(2-fluorophenyl)ureido)-1H-pyrazol-1-yl)-N-(3-methyl-1-(2-(piperidin-1-yl)ethyl)-1H-pyrazol-4-yl)benzamide

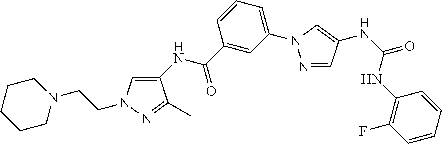 [SMILES: Cc1nn(CCN2CCCCC2)cc1NC(=O)c1cccc(-n2cc(NC(=O)Nc3ccccc3F)cn2)c1]

Procedures in Scheme 1 were utilized to synthesize this compound. ¹H NMR (400 MHz, MeOD) δ 8.48 (s, 1H), 8.32 (t, J=1.8 Hz, 1H), 8.10-8.03 (m, 2H), 7.98 (ddd, J=8.1, 2.2, 0.9 Hz, 1H), 7.87 (d, J=8.2 Hz, 1H), 7.77 (s, 1H), 7.69-7.61 (m, 1H), 7.19-7.11 (m, 2H), 7.07 (dddd, J=8.9, 7.1, 5.1, 1.7 Hz, 1H), 4.55 (t, J=6.0 Hz, 2H), 3.69-3.52 (m, 4H), 3.04 (t, J=12.1 Hz, 2H), 2.31 (s, 3H), 1.99 (d, J=14.4 Hz, 2H), 1.92-1.70 (s, 3H), 1.56 (d, J=12.0 Hz, 1H). LC-MS: 531 (M+H).

### Example I-116

### N-(3-methyl-1-(2-(piperidin-1-yl)ethyl)-1H-pyrazol-4-yl)-3-(4-(3-(m-tolyl)ureido)-1 H-pyrazol-1-yl)benzamide

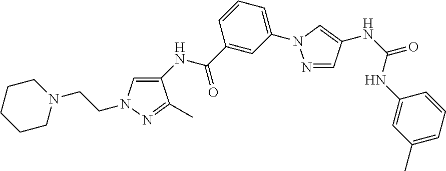 [SMILES: Cc1cccc(NC(=O)Nc2cnn(-c3cccc(C(=O)Nc4cn(CCN5CCCCC5)nc4C)c3)c2)c1]

Procedures in Scheme 1 were utilized to synthesize this compound. ¹H NMR (400 MHz, DMSO) δ 9.99 (s, 1H), 9.37 (s, 1H), 8.87 (s, 2H), 8.65-8.53 (m, 1H), 8.40-8.30 (m, 1H), 8.12 (s, 1H), 8.01 (d, J=8.1 Hz, 1H), 7.89-7.78 (m, 2H), 7.71-7.58 (m, 1H), 7.35 (s, 1H), 7.25 (d, J=8.0 Hz, 1H), 7.15 (t, J=7.8 Hz, 1H), 6.78 (d, J=7.4 Hz, 1H), 4.47 (t, J=6.7 Hz, 2H), 3.50 (d, J=20.7 Hz, 6H), 3.04-2.86 (m, 3H), 2.28 (s, 3H), 2.23 (s, 2H), 1.81 (s, 2H), 1.65 (d, J=14.2 Hz, 2H). LC-MS: 527 (M+H).

### Example I-117

### 3-(4-(3-(3-methoxyphenyl)ureido)-1H-pyrazol-1-yl)-N-(5-methyl-1-(2-(piperidin-1-yl)ethyl)-1H-pyrazol-4-yl)benzamide

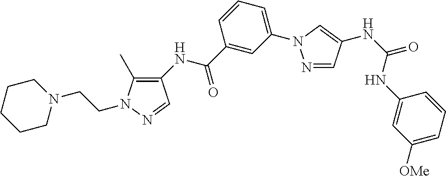 [SMILES: COc1cccc(NC(=O)Nc2cnn(-c3cccc(C(=O)Nc4cnn(CCN5CCCCC5)c4C)c3)c2)c1]

Procedures in Scheme 1 were utilized to synthesize this compound. LC-MS: 543 (M+H).

### Example I-118

### 3-(4-(3-(3-methoxyphenyl)ureido)-1H-pyrazol-1-yl)-N-(3-methyl-1-(2-(piperidin-1-yl)ethyl)-1H-pyrazol-4-yl)benzamide

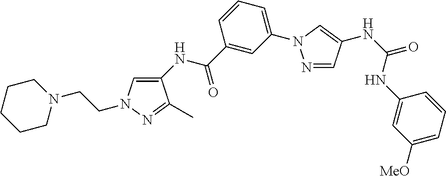 [SMILES: COc1cccc(NC(=O)Nc2cnn(-c3cccc(C(=O)Nc4cn(CCN5CCCCC5)nc4C)c3)c2)c1]

Procedures in Scheme 1 were utilized to synthesize this compound. LC-MS: 543 (M+H).

### Example I-119

### 3-(4-(3-(2-chlorophenyl)ureido)-1H-pyrazol-1-yl)-N-(1-(2-(diethylamino)ethyl)-1 H-pyrazol-4-yl)benzamide

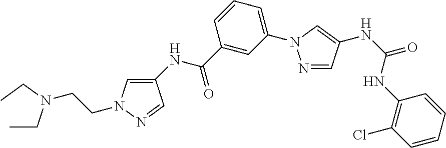 [SMILES: CCN(CC)CCn1cc(NC(=O)c2cccc(-n3cc(NC(=O)Nc4ccccc4Cl)cn3)c2)cn1]

Procedures in Scheme 1 were utilized to synthesize this compound. ¹H NMR (400 MHz, MeOD) δ 8.48 (d, J=0.5 Hz, 1H), 8.32 (t, J=1.9 Hz, 1H), 8.28 (s, 1H), 8.14 (dd, J=8.3, 1.5 Hz, 1H), 8.01-7.93 (m, 1H), 7.86 (dd, J=5.4, 3.9 Hz, 1H), 7.78 (d, J=14.9 Hz, 2H), 7.65 (t, J=8.0 Hz, 1H), 7.43 (dd, J=8.0, 1.4 Hz, 1H), 7.33-7.24 (m, 1H), 7.10-6.98 (m, 1H), 4.61 (t, J=5.9 Hz, 2H), 3.72 (t, J=5.9 Hz, 2H), 3.32-3.28 (m, 4H), 1.34 (t, J=7.3 Hz, 6H). LC-MS: 521 (M+H).

### Example I-120

### 3-(4-(3-cyclopentylureido)-1H-pyrazol-1-yl)-N-(3-methyl-1-(2-(pyrrolidin-1-yl)ethyl)-1H-pyrazol-4-yl)benzamide

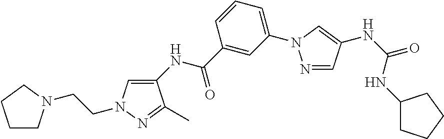 [SMILES: Cc1nn(CCN2CCCC2)cc1NC(=O)c1cccc(-n2cc(NC(=O)NC3CCCC3)cn2)c1]

Procedures in Scheme 1 were utilized to synthesize this compound. LC-MS: 491 (M+H).

### Example I-121

### 3-(4-(3-(2-chlorophenyl)thioureido)-1H-pyrazol-1-yl)-N-(1-(2-(diethylamino)ethyl)-1H-pyrazol-4-yl)benzamide

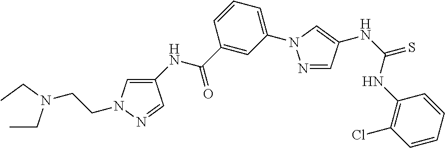 [SMILES: CCN(CC)CCn1cc(NC(=O)c2cccc(-n3cc(NC(=S)Nc4ccccc4Cl)cn3)c2)cn1]

Procedures in Scheme 1 were utilized to synthesize this compound. LC-MS: 537 (M+H).

### Example I-122

### 3-(4-(3-cyclopentylureido)-1H-pyrazol-1-yl)-N-(1-(2-(diethylamino)ethyl)-1H-pyrazol-4-yl)benzamide

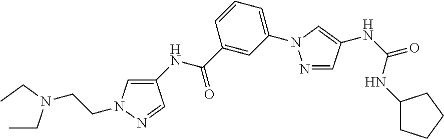 [SMILES: CCN(CC)CCn1cc(NC(=O)c2cccc(-n3cc(NC(=O)NC4CCCC4)cn3)c2)cn1]

Procedures in Scheme 1 were utilized to synthesize this compound. LC-MS: 479 (M+H).

### Example I-123

### 3-(4-(3-(2-chlorophenyl)ureido)-1H-pyrazol-1-yl)-N-(1-(piperidin-4-ylmethyl)-1H-pyrazol-4-yl)benzamide

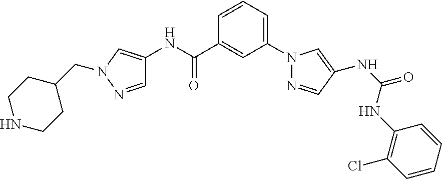 [SMILES: O=C(Nc1cnn(-c2cccc(C(=O)Nc3cnn(CC4CCNCC4)c3)c2)c1)Nc1ccccc1Cl]

Procedures in Scheme 1 were utilized to synthesize this compound. ¹H NMR (400 MHz, DMSO) δ 9.65 (s, 1H), 8.84 (dd, J=12.7, 6.7 Hz, 2H), 8.58 (s, 1H), 8.48 (s, 2H), 8.27 (t, J=1.7 Hz, 1H), 8.19 (dd, J=8.3, 1.5 Hz, 1H), 8.03-7.91 (m, 1H), 7.87-7.80 (m, 1H), 7.77 (d, J=7.9 Hz, 1H), 7.58 (t, J=7.9 Hz, 1H), 7.46 (dd, J=8.0, 1.5 Hz, 1H), 7.36-7.26 (m, 1H), 7.10-6.99 (m, 1H), 3.35-3.17 (m, 4H), 2.89-2.79 (m, 2H), 1.85 (t, J=11.5 Hz, 3H), 1.39 (q, J=10.9 Hz, 2H). LC-MS: 519 (M+H).

### Example I-124

### N-(1-(piperidin-4-ylmethyl)-1H-pyrazol-4-yl)-3-(4-(3-(o-tolyl)ureido)-1H-pyrazol-1-yl)benzamide

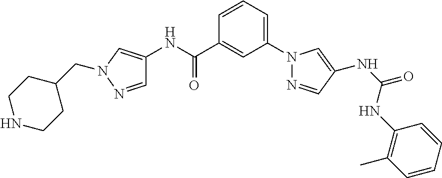 [SMILES: Cc1ccccc1NC(=O)Nc1cnn(-c2cccc(C(=O)Nc3cnn(CC4CCNCC4)c3)c2)c1]

Procedures in Scheme 1 were utilized to synthesize this compound. ¹H NMR (400 MHz, DMSO) δ 10.55 (d, J=9.0 Hz, 1H), 8.95 (s, 1H), 8.50 (s, 1H), 8.28 (d, J=17.6 Hz, 1H), 8.01 (d, J=3.5 Hz, 1H), 7.98-7.87 (m, 2H), 7.81-7.67 (m, 3H), 7.63-7.49 (m, 2H), 7.09 (dd, J=15.8, 7.8 Hz, 2H), 6.88 (dd, J=8.0, 6.8 Hz, 1H), 3.93 (t, J=8.9 Hz, 3H), 3.86 (s, 2H), 3.38 (s, 1H), 2.60 (s, 2H), 2.18 (s, 3H), 1.91 (s, 1H), 1.39 (d, J=11.9 Hz, 2H), 1.08-0.89 (m, 2H). LC-MS: 499 (M+H).

### Example I-125

### 3-(4-(3-(3-methoxyphenyl)ureido)-1H-pyrazol-1-yl)-N-(1-(piperidin-4-ylmethyl)-1H-pyrazol-4-yl)benzamide

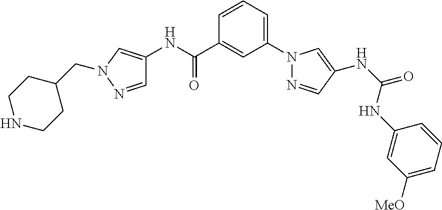 [SMILES: COc1cccc(NC(=O)Nc2cnn(-c3cccc(C(=O)Nc4cnn(CC5CCNCC5)c4)c3)c2)c1]

Procedures in Scheme 1 were utilized to synthesize this compound. LC-MS: 515 (M+H).

### Example I-126

### 3-(4-(3-(2-chlorophenyl)ureido)-1H-pyrazol-1-yl)-N-(1-(pyrrolidin-3-yl)-1H-pyrazol-4-yl)benzamide

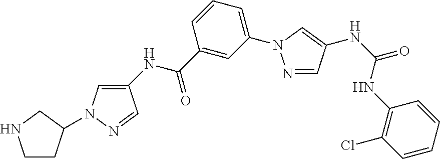 [SMILES: O=C(Nc1cnn(-c2cccc(C(=O)Nc3cnn(C4CCNC4)c3)c2)c1)Nc1ccccc1Cl]

Procedures in Scheme 1 were utilized to synthesize this compound. LC-MS: 491 (M+H).

### Example I-127

### N-(1-(pyrrolidin-3-yl)-1H-pyrazol-4-yl)-3-(4-(3-(o-tolyl)ureido)-1H-pyrazol-1-yl)benzamide

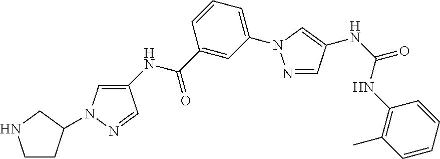 [SMILES: Cc1ccccc1NC(=O)Nc1cnn(-c2cccc(C(=O)Nc3cnn(C4CCNC4)c3)c2)c1]

Procedures in Scheme 1 were utilized to synthesize this compound. LC-MS: 471 (M+H).

### Example I-128

### 3-(4-(3-(3-methoxyphenyl)ureido)-1H-pyrazol-1-yl)-N-(1-(pyrrolidin-3-yl)-1H-pyrazol-4-yl)benzamide

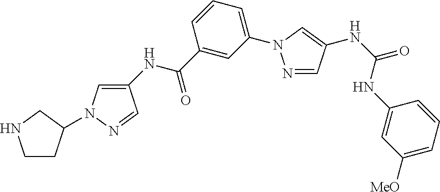 [SMILES: COc1cccc(NC(=O)Nc2cnn(-c3cccc(C(=O)Nc4cnn(C5CCNC5)c4)c3)c2)c1]

Procedures in Scheme 1 were utilized to synthesize this compound. LC-MS: 487 (M+H).

### Example I-129

### 3-(4-(3-(2-chlorophenyl)ureido)-1H-pyrazol-1-yl)-N-(1-(4-(diethylamino)butyl)-1H-pyrazol-4-yl)benzamide

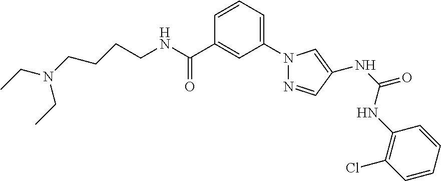 [SMILES: CCN(CC)CCCCNC(=O)c1cccc(-n2cc(NC(=O)Nc3ccccc3Cl)cn2)c1]

Procedures in Scheme 1 were utilized to synthesize this compound. LC-MS: 483 (M+H).

### Example I-130

### N-(1-(4-(diethylamino)butyl)-1H-pyrazol-4-yl)-3-(4-(3-(o-tolyl)ureido)-1H-pyrazol-1-yl)benzamide

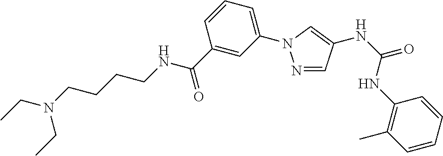 [SMILES: CCN(CC)CCCCNC(=O)c1cccc(-n2cc(NC(=O)Nc3ccccc3C)cn2)c1]

Procedures in Scheme 1 were utilized to synthesize this compound. LC-MS: 463 (M+H).

### Example I-131

### N-(1-(4-(diethylamino)butyl)-1H-pyrazol-4-yl)-3-(4-(3-(3-methoxyphenyl)ureido)-1H-pyrazol-1-yl)benzamide

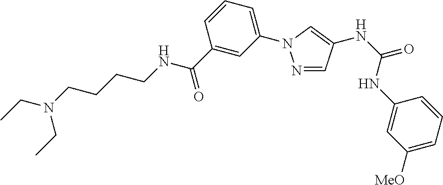 [SMILES: CCN(CC)CCCCNC(=O)c1cccc(-n2cc(NC(=O)Nc3cccc(OC)c3)cn2)c1]

Procedures in Scheme 1 were utilized to synthesize this compound. LC-MS: 479 (M+H).

### Example I-132

### 3-(4-(3-(4-chlorophenyl)ureido)-1H-pyrazol-1-yl)-N-(1-(2-(diethylamino)ethyl)-1 H-pyrazol-4-yl)benzamide

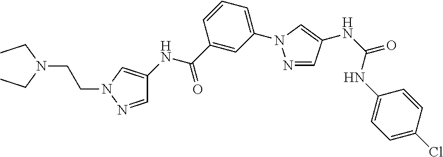 [SMILES: CCN(CC)CCn1cc(NC(=O)c2cccc(-n3cc(NC(=O)Nc4ccc(Cl)cc4)cn3)c2)cn1]

Procedures in Scheme 1 were utilized to synthesize this compound. LC-MS: 520 (M+H).

### Example I-133

### N-(2-chlorobenzyl)-1-(3-((1-isopropyl-1H-pyrazol-4-yl)carbamoyl)phenyl)-1H-pyrazole-4-carboxamide

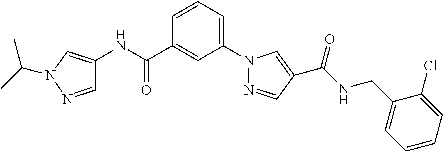 [SMILES: CC(C)n1cc(NC(=O)c2cccc(-n3cc(C(=O)NCc4ccccc4Cl)cn3)c2)cn1]

Procedures in Scheme 5 were utilized to synthesize this compound. LC-MS: 463 (M+H).

### Example I-134

### N-(2-fluorobenzyl)-1-(3-((1-isopropyl-1H-pyrazol-4-yl)carbamoyl)phenyl)-1H-pyrazole-4-carboxamide

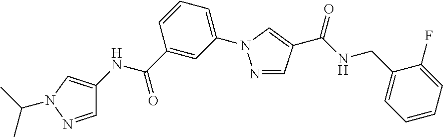 [SMILES: CC(C)n1cc(NC(=O)c2cccc(-n3cc(C(=O)NCc4ccccc4F)cn3)c2)cn1]

Procedures in Scheme 5 were utilized to synthesize this compound. LC-MS: 447 (M+H).

### Example I-135

### 1-(3-((1-isopropyl-1H-pyrazol-4-yl)carbamoyl)phenyl)-N-(3-methylbenzyl)-1H-pyrazole-4-carboxamide

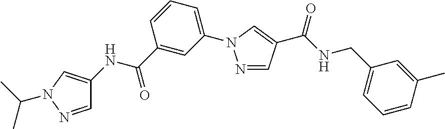 [SMILES: Cc1cccc(CNC(=O)c2cnn(-c3cccc(C(=O)Nc4cnn(C(C)C)c4)c3)c2)c1]

Procedures in Scheme 5 were utilized to synthesize this compound. LC-MS: 443 (M+H).

### Example I-141

### 3-(4-(3-(2-chlorophenyl)ureido)-1H-pyrazol-1-yl)-N-(3-methyl-1H-indazol-5-yl)benzamide

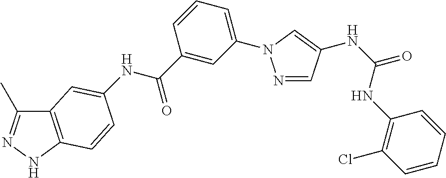 [SMILES: Cc1n[nH]c2ccc(NC(=O)c3cccc(-n4cc(NC(=O)Nc5ccccc5Cl)cn4)c3)cc12]

Procedures in Scheme 1 were utilized to synthesize this compound. ¹H NMR (400 MHz, DMSO) δ 12.62 (s, 1H), 10.44 (s, 1H), 9.44 (s, 1H), 8.63 (s, 1H), 8.39 (s, 2H), 8.29-8.16 (m, 2H), 8.04 (dd, J=8.1, 1.3 Hz, 1H), 7.87 (dd, J=12.0, 4.2 Hz, 2H), 7.65 (dd, J=12.6, 4.9 Hz, 2H), 7.51-7.41 (m, 2H), 7.38-7.26 (m, 1H), 7.09-6.99 (m, 1H), 3.49 (d, J=4.5 Hz, 3H). LC-MS: 486 (M+H).

### Example I-142

### 1-(2-chlorophenyl)-3-(1-(3-(4-morpholinopiperidine-1-carbonyl)phenyl)-1H-pyrazol-4-yl)urea

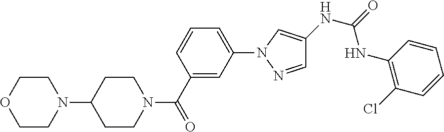 [SMILES: O=C(Nc1cnn(-c2cccc(C(=O)N3CCC(N4CCOCC4)CC3)c2)c1)Nc1ccccc1Cl]

Procedures in Scheme 1 were utilized to synthesize this compound. ¹H NMR (400 MHz, DMSO) δ 9.93 (s, 1H), 9.43 (s, 1H), 8.52 (s, 1H), 8.40 (s, 1H), 8.18 (dd, J=8.3, 1.3 Hz, 1H), 7.92 (d, J=8.3 Hz, 1H), 7.84 (s, 2H), 7.57 (t, J=7.9 Hz, 1H), 7.46 (dd, J=8.0, 1.3 Hz, 1H), 7.30 (t, J=8.0 Hz, 2H), 7.10-6.96 (m, 1H), 4.64 (s, 1H), 4.01 (d, J=11.1 Hz, 2H), 3.69 (d, J=12.2 Hz, 3H), 3.44 (s, 3H), 3.13 (s, 3H), 2.81 (s, 1H), 2.24-1.94 (m, 2H), 1.61 (s, 2H). LC-MS: 509 (M+H).

### Example I-143

### 3-(4-(3-(2-chlorophenyl)ureido)-1H-pyrazol-1-yl)-N-((1-methylpiperidin-4-yl)methyl)benzamide

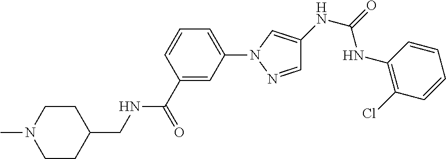 [SMILES: CN1CCC(CNC(=O)c2cccc(-n3cc(NC(=O)Nc4ccccc4Cl)cn3)c2)CC1]

Procedures in Scheme 1 were utilized to synthesize this compound. LC-MS: 467 (M+H).

### Example I-144

### 3-(4-(3-(2-chlorophenyl)ureido)-1H-pyrazol-1-yl)-N-(3-(pyrrolidin-1-yl)propyl)benzamide

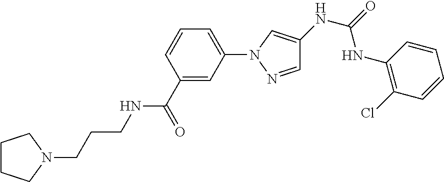 [SMILES: O=C(Nc1cnn(-c2cccc(C(=O)NCCCN3CCCC3)c2)c1)Nc1ccccc1Cl]

Procedures in Scheme 1 were utilized to synthesize this compound. ¹H NMR (400 MHz, MeOD) δ 8.45 (s, 1H), 8.23 (t, J=1.8 Hz, 1H), 8.13 (dd, J=8.3, 1.5 Hz, 1H), 8.00-7.89 (m, 1H), 7.84-7.71 (m, 2H), 7.60 (t, J=8.0 Hz, 1H), 7.43 (dd, J=8.0, 1.4 Hz, 1H), 7.30 (ddd, J=8.3, 7.5, 1.5 Hz, 1H), 7.12-6.92 (m, 1H), 3.78-3.62 (m, 2H), 3.55 (t, J=6.5 Hz, 2H), 3.29 (d, J=7.9 Hz, 2H), 3.11 (dd, J=10.8, 7.9 Hz, 2H), 2.23-2.13 (m, 2H), 2.13-1.90 (m, 4H). LC-MS: 467 (M+H).

### Example I-145

### 3-(4-(3-(2-chlorophenyl)ureido)-1H-pyrazol-1-yl)-N-(3-morpholinopropyl)benzamide

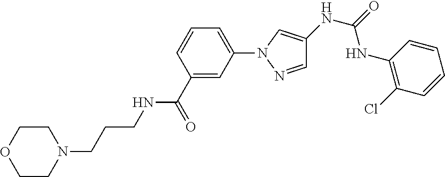 [SMILES: O=C(Nc1cnn(-c2cccc(C(=O)NCCCN3CCOCC3)c2)c1)Nc1ccccc1Cl]

Procedures in Scheme 1 were utilized to synthesize this compound. LC-MS: 483 (M+H).

### Example I-146

### 3-(4-(3-(2-chlorophenyl)ureido)-1H-pyrazol-1-yl)-N-(piperidin-4-ylmethyl)benzamide

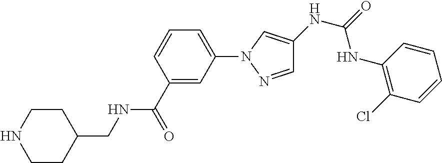 [SMILES: O=C(Nc1cnn(-c2cccc(C(=O)NCC3CCNCC3)c2)c1)Nc1ccccc1Cl]

Procedures in Scheme 1 were utilized to synthesize this compound. LC-MS: 452 (M+H).

### Example I-147

### 3-(4-(3-(2-chlorophenyl)ureido)-1H-pyrazol-1-yl)-N-((tetrahydro-2H-pyran-4-yl)methyl)benzamide

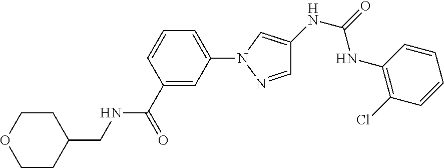 [SMILES: O=C(Nc1cnn(-c2cccc(C(=O)NCC3CCOCC3)c2)c1)Nc1ccccc1Cl]

Procedures in Scheme 1 were utilized to synthesize this compound. ¹H NMR (400 MHz, DMSO) δ 9.41 (s, 1H), 8.72 (d, J=5.8 Hz, 1H), 8.57 (s, 1H), 8.38 (s, 1H), 8.25-8.19 (m, 2H), 7.97 (d, J=8.1 Hz, 1H), 7.82 (s, 1H), 7.75 (d, J=7.8 Hz, 1H), 7.57 (t, J=7.9 Hz, 1H), 7.47 (dd, J=8.0, 1.5 Hz, 1H), 7.35-7.27 (m, 1H), 7.09-7.01 (m, 1H), 3.86 (d, J=10.5 Hz, 2H), 3.32-3.23 (m, 4H), 3.19 (dd, J=11.5, 5.4 Hz, 2H), 1.83 (s, 1H), 1.62 (d, J=11.1 Hz, 2H), 1.22 (dd, J=12.7, 4.3 Hz, 2H). LC-MS: 454 (M+H).

### Example I-148

### 3-(4-(3-(2-chlorophenyl)ureido)-1H-pyrazol-1-yl)-N-(2-(diethylamino)ethyl)benzamide

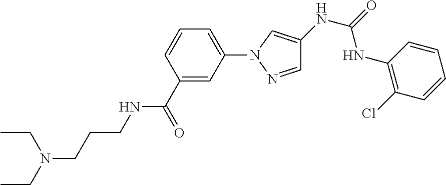 [SMILES: CCN(CC)CCCNC(=O)c1cccc(-n2cc(NC(=O)Nc3ccccc3Cl)cn2)c1]

Procedures in Scheme 1 were utilized to synthesize this compound. ¹H NMR (400 MHz, CDCl₃) δ 7.67 (s, 1H), 7.46 (s, 1H), 7.35 (dd, J=8.2, 1.4 Hz, 1H), 7.16 (d, J=7.3 Hz, 1H), 7.04-6.92 (m, 2H), 6.83 (t, J=7.9 Hz, 1H), 6.64 (dd, J=8.0, 1.4 Hz, 1H), 6.58-6.45 (m, 1H), 6.32-6.22 (m, 1H), 2.76 (t, J=6.4 Hz, 2H), 2.48 (dt, J=18.0, 8.9 Hz, 6H), 1.38-1.20 (m, 2H), 0.56 (t, J=7.3 Hz, 6H). LC-MS: 469 (M+H).

### Example I-149

### N-(2-(diethylamino)ethyl)-3-(4-(3-(o-tolyl)ureido)-1H-pyrazol-1-yl)benzamide

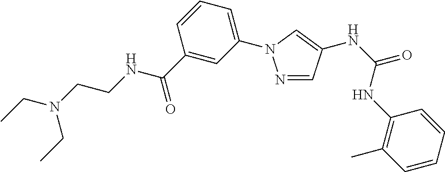 [SMILES: CCN(CC)CCNC(=O)c1cccc(-n2cc(NC(=O)Nc3ccccc3C)cn2)c1]

Procedures in Scheme 1 were utilized to synthesize this compound. LC-MS: 435 (M+H).

### Example I-150

### 3-(4-(3-(4-chlorophenyl)ureido)-1H-pyrazol-1-yl)-N-(2-(diethylamino)ethyl)benzamide

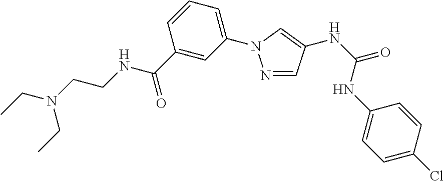 [SMILES: CCN(CC)CCNC(=O)c1cccc(-n2cc(NC(=O)Nc3ccc(Cl)cc3)cn2)c1]

Procedures in Scheme 1 were utilized to synthesize this compound. LC-MS: 455 (M+H).

### Example I-151

### N-(2-(diethylamino)ethyl)-3-(4-(3-(2-fluorophenyl)ureido)-1H-pyrazol-1-yl)benzamide

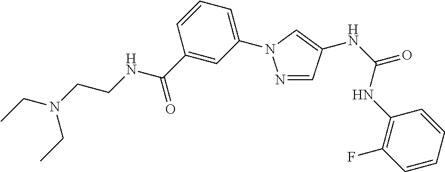 [SMILES: CCN(CC)CCNC(=O)c1cccc(-n2cc(NC(=O)Nc3ccccc3F)cn2)c1]

Procedures in Scheme 1 were utilized to synthesize this compound. LC-MS: 439 (M+H).

### Example I-152

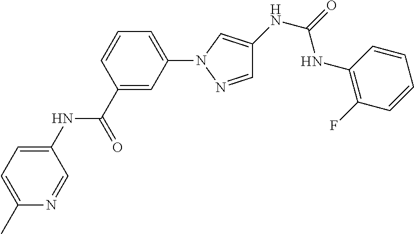 [SMILES: Cc1ccc(NC(=O)c2cccc(-n3cc(NC(=O)Nc4ccccc4F)cn3)c2)cn1]

### Example I-153

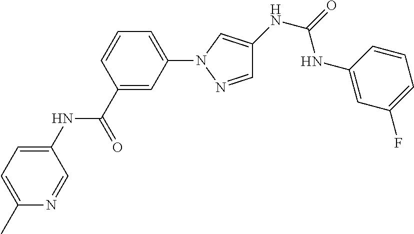 [SMILES: Cc1ccc(NC(=O)c2cccc(-n3cc(NC(=O)Nc4cccc(F)c4)cn3)c2)cn1]

### Example I-154

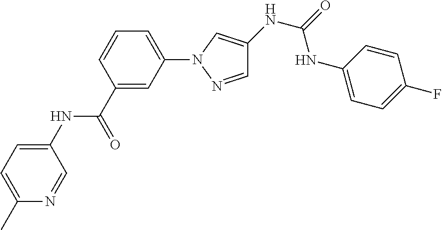 [SMILES: Cc1ccc(NC(=O)c2cccc(-n3cc(NC(=O)Nc4ccc(F)cc4)cn3)c2)cn1]

### Example I-155

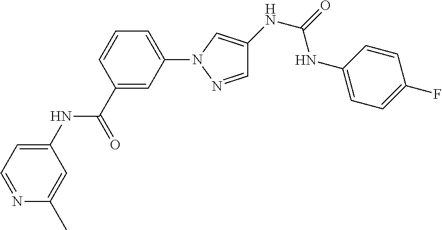 [SMILES: Cc1cc(NC(=O)c2cccc(-n3cc(NC(=O)Nc4ccc(F)cc4)cn3)c2)ccn1]

### Example I-156

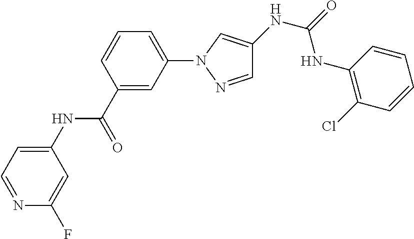 [SMILES: O=C(Nc1cnn(-c2cccc(C(=O)Nc3ccnc(F)c3)c2)c1)Nc1ccccc1Cl]

### Example I-157

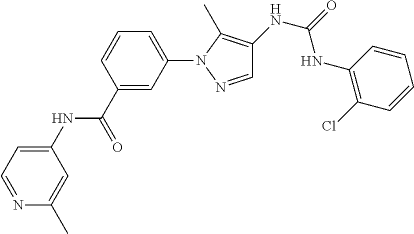 [SMILES: Cc1cc(NC(=O)c2cccc(-n3ncc(NC(=O)Nc4ccccc4Cl)c3C)c2)ccn1]

### Example I-158

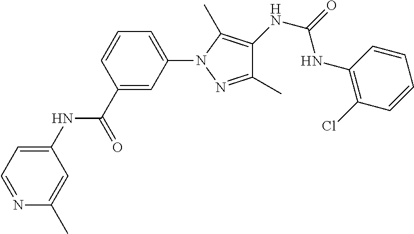 [SMILES: Cc1cc(NC(=O)c2cccc(-n3nc(C)c(NC(=O)Nc4ccccc4Cl)c3C)c2)ccn1]

### Example I-159

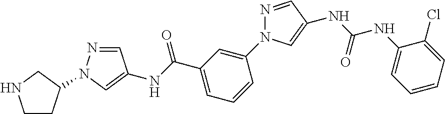

### Example I-160

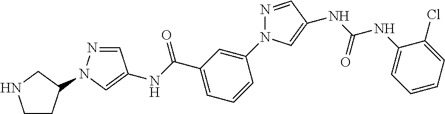

### Example I-161

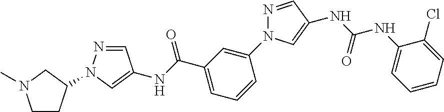

### Example I-162

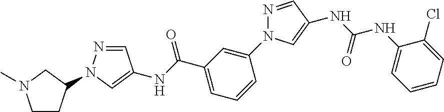

### Example I-163

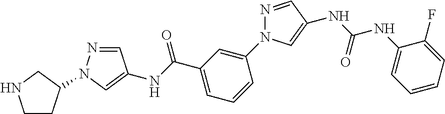

### Example I-164

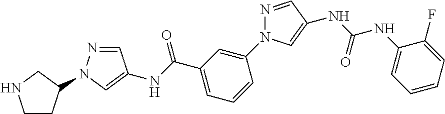

### Example I-165

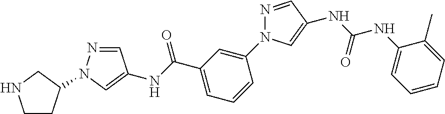

### Example I-166

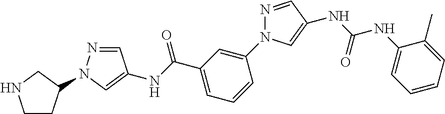

### Example I-167

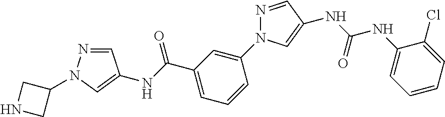 [SMILES: O=C(Nc1cnn(-c2cccc(C(=O)Nc3cnn(C4CNC4)c3)c2)c1)Nc1ccccc1Cl]

### Example I-168

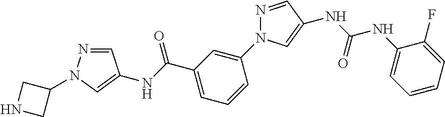 [SMILES: O=C(Nc1cnn(-c2cccc(C(=O)Nc3cnn(C4CNC4)c3)c2)c1)Nc1ccccc1F]

### Example I-169

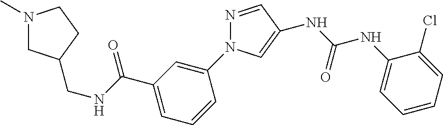 [SMILES: CN1CCC(CNC(=O)c2cccc(-n3cc(NC(=O)Nc4ccccc4Cl)cn3)c2)C1]

### Example I-170

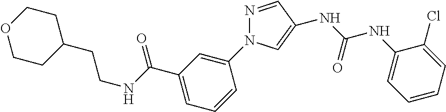 [SMILES: O=C(Nc1cnn(-c2cccc(C(=O)NCCC3CCOCC3)c2)c1)Nc1ccccc1Cl]

### Example I-171

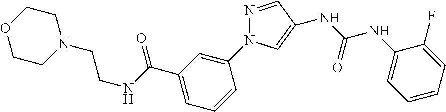 [SMILES: O=C(Nc1cnn(-c2cccc(C(=O)NCCN3CCOCC3)c2)c1)Nc1ccccc1F]

### Example I-172

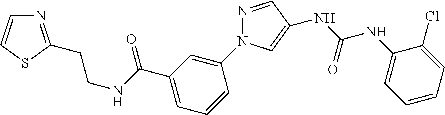 [SMILES: O=C(Nc1cnn(-c2cccc(C(=O)NCCc3nccs3)c2)c1)Nc1ccccc1Cl]

### Example I-173

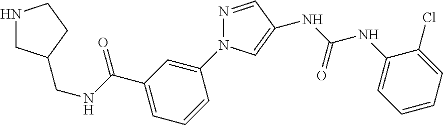 [SMILES: O=C(Nc1cnn(-c2cccc(C(=O)NCC3CCNC3)c2)c1)Nc1ccccc1Cl]

### Example I-174

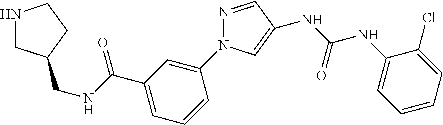 [SMILES: O=C(Nc1cnn(-c2cccc(C(=O)NC[C@@H]3CCNC3)c2)c1)Nc1ccccc1Cl]

### Example I-175

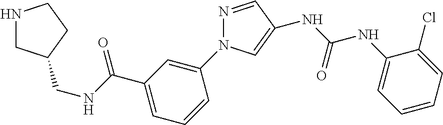 [SMILES: O=C(Nc1cnn(-c2cccc(C(=O)NC[C@H]3CCNC3)c2)c1)Nc1ccccc1Cl]

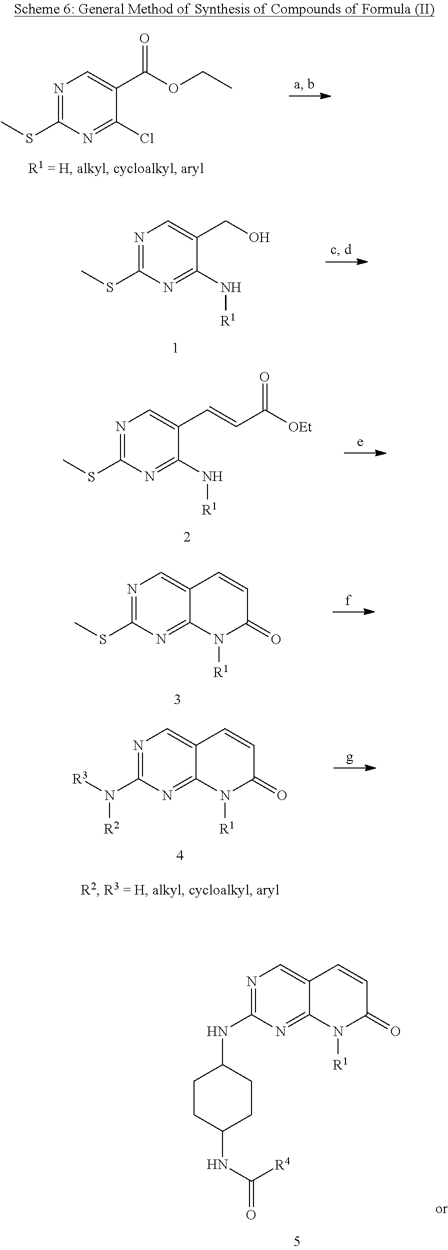

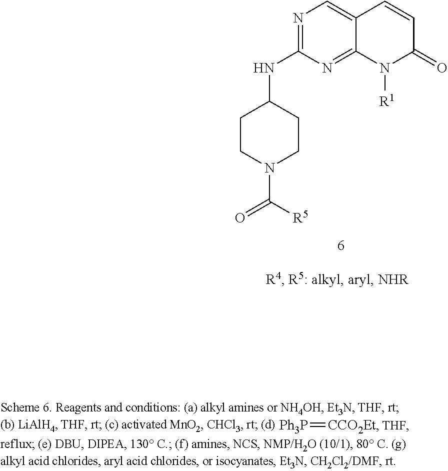

The mixture of ethyl 4-chloro-2-methylthio-5-pyrimidine carboxylate (12.9 mmol), Et₃N (39.9 mmol), and amines (39.9 mmol) in THF (200 mL) was stirred at room temperature for 3 h. After evaporation of the resulting residue, the mixture was diluted with H₂O and extracted with EtOAc. After evaporation, the crude mixture was purified through silicagel to give amine substituted pyrimidine. To this amine substituted pyrimidine (6.9 mmol) in THF (100 mL) was added LiAlH₄ (13.8 mmol) at 0° C. After stirring at room temperature for 8 h, H₂O (1.81 mL), 2N NaOH (5.9 mL), and H₂O (1.81 mL) were added sequentially. The resulting solids were filtered off and the combined organic layer was evaporated and purified by column chromatography to afford intermediate alcohol 1 (65% yield for steps).

The mixture of intermediate 1 (9.7 mmol) and activated MnO₂ (72.7 mmol) in CHCl₃ (150 mL) was stirred at room temperature for 6 h. After filtering off the black solid, the resulting aldehyde was dissolved in THF (100 mL) and Ph₃P═CCO₂Et (12.9 mmol) was added. The reaction mixture was allowed to react at 65° C. for 1 h. The resulting mixture was evaporated, dissolved in water, extracted with EtOAc, and purified by column chromatography to afford ester 2 (71% yield for steps).

After adding DBU (51 mmol) and DIPEA (51 mmol) to intermediate ester 2 (17.3 mmol), the mixture was stirred at 130° C. for 12 h, evaporated, and purified by silica gel to afford intermediate pyrido[2,3-d]pyrimidin-7-ones 3 (67% yield).

After stirring the mixture of intermediate compound 3 (2.0 mmol), NCS (2.1 mmol) in NMP (5 mL) and H₂O (0.5 mL) at 80° C. for 15 min, amines (4.0 mmol) was added, stirred at 80° C. for 8 h, and purified by prep HPLC to give intermediate compound 4 (60% yield).

The mixture of intermediate compound 4 (0.2 mmol), Et₃N (0.4 mmol) in CH₂Cl₂ (1 mL) and DMF (0.1 mL) were further functionalized by the addition of alkyl acid chlorides, aryl acid chlorides, or isocyanates (0.2 mmol) and purified by prep HPLC to give compounds of general formulas 5 or 6 (70% yield).

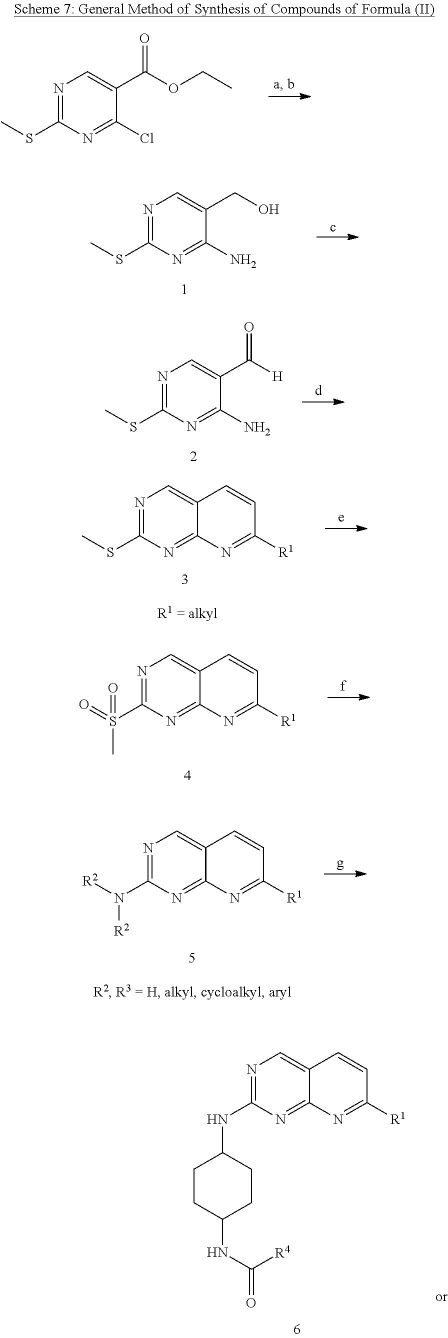

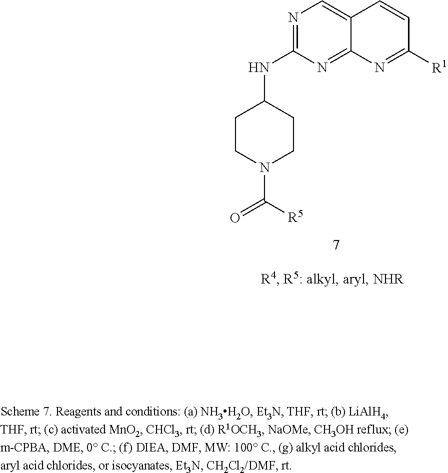

The mixture of ethyl 4-chloro-2-methylthio-5-pyrimidine carboxylate (20.0 mmol), Et₃N (10 ml), and 50% aq NH₃.H₂O (8 ml) in THF (100 mL) was stirred at room temperature for 4 h. After evaporation of the resulting residue, the mixture was diluted with H₂O and extracted with EtOAc. After evaporation, get the crude amino substituted pyrimidine. To this crude amino substituted pyrimidine in THF (100 mL) was added dropwise LiAlH₄ (11.0 mmol) in 30 ml Et₂O at 0° C. After stirring at room temperature for 8 h, H₂O (3.0 mL), 2N NaOH (10 mL), and H₂O (3.0 mL) were added sequentially. The crude product was purified by column chromatography to afford intermediate alcohol 1 (73% yield for steps).

The mixture of intermediate compound 1 (14.6 mmol) and activated MnO₂ (80.0 mmol) in CH₂Cl₂ (200 mL) was stirred at room temperature for 6 h. After filtering off the black solid, the resulting intermediate aldehyde 2 was dissolved in CH₃OH (100 mL). The NaOMe (15.0 mmol) and 1-cyclopropylethan-1-one (16.0 mmol) were added at room temperature. The reaction mixture was allowed to react at reflux for 4 h. The resulting mixture was evaporated, dissolved in water, extracted with EtOAc, and purified by column chromatography to afford intermediate product 3 (65% yield for steps).

The resulting intermediate 3 (9.5 mmol) was dissolved in CH₂Cl₂ (50 mL) and m-CPBA (20.0 mmol) was added and stirred at room temperature for 12 h. The resulting mixture was evaporated, dissolved in water, extracted with EtOAc, and purified by column chromatography to afford intermediate product 4 (67% yield).

The mixture of intermediate compound 4 (1.0 mmol) and DIEA (3.0 mmol) in DMF (5 mL) was heated at 100° C. for 2 h under microwave reaction, and purified by prep HPLC to give intermediate compound 5 (65% yield).

The mixture of intermediate compound 5 (0.2 mmol), Et₃N (0.4 mmol) in CH₂Cl₂ (1 mL) and DMF (0.1 mL) were further functionalized by the addition of alkyl acid chlorides, aryl acid chlorides, or isocyanates (0.2 mmol) and purified by prep HPLC to give compounds of general formulas 6 or 7 (62% yield).

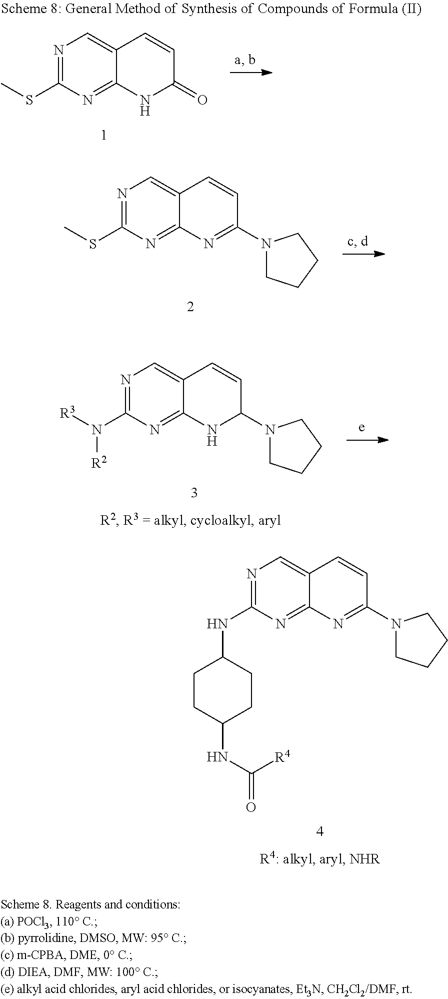

The previously obtained intermediate 1 (10.0 mmol) was dissolved in POCl₃ (50 mL) and stirred at 110° C. for 4 h. The resulting mixture was evaporated, and the ice water was added at 0° C., extracted with EtOAc, and the solvent was evaporated to afford intermediate. The mixture of intermediate and pyrrolidine (12.0 mmol) in DMSO (15.0 mL) was heated at 95° C. for 2 h under microwave. The resulting mixture was evaporated, washed by water, extracted with EtOAc, and purified by column chromatography to afford intermediate product 2 (65% yield).

The intermediate 2 (5.0 mmol) was dissolved in CH₂Cl₂ (50 mL) and m-CPBA (12.0 mmol) was added and stirred at room temperature for 12 h. The resulting mixture was evaporated, dissolved in water, extracted with EtOAc, and purified by column chromatography to afford oxidation product (75% yield). The mixture of resulting oxidation product (1.0 mmol) and DIEA (3.0 mmol) in DMF (5 mL) was heated at 100° C. for 2 h under microwave reaction, and purified by prep HPLC to give intermediate compound 3 (67% yield).

The mixture of intermediate compound 3 (0.2 mmol), Et₃N (0.4 mmol) in CH₂Cl₂ (1 mL) and DMF (0.1 mL) was further functionalized by the addition of alkyl acid chlorides, aryl acid chlorides, or isocyanates (0.2 mmol) and purified by prep HPLC to give compounds of general formula 4 (60% yield).

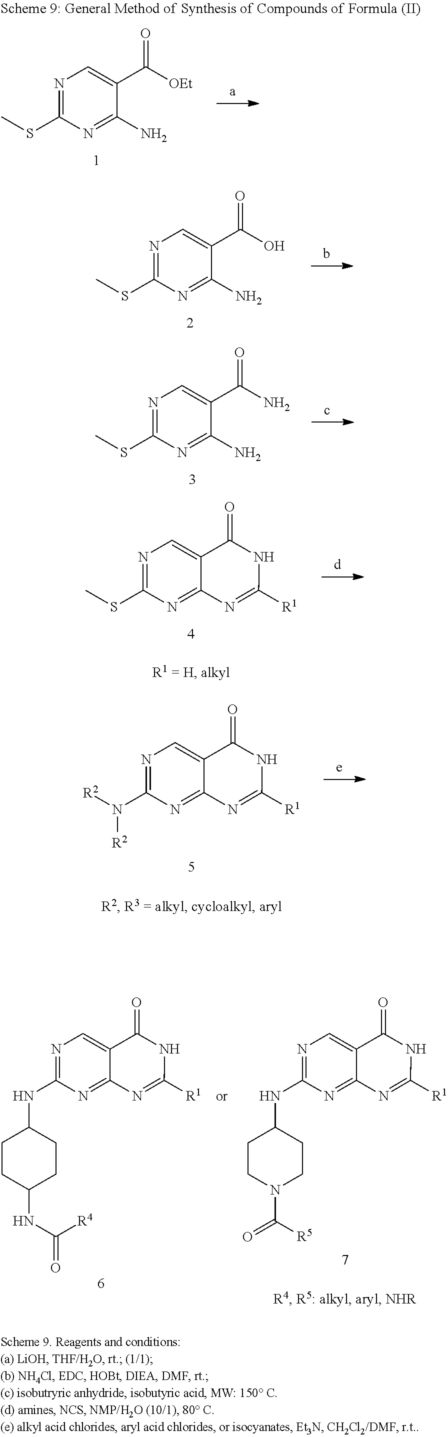

A mixture of ethyl 4-amino-2-(methylthio)pyrimidine-5-carboxylate 1 (5 mmol) and LiOH (2.0 g) was stirred in THF (30 mL)/water (30 mL) at RT for 2-3 hours. The resulting homogenous reaction was acidified with 2M HCl (20 mL) and extracted with EtOAc (2×100 mL). The combined organic extracts were washed with water (100 mL), dried (Na₂SO₄) and evaporated. The crude residue was dried under vacuum and used without further purification.

A solution of crude acid 2 in CH₂Cl₂ (20 mL) was added EDC (10 mmol), HOBt (10 mmol) and diisopropyl ethyl amine (15 mmol) and stirred for 30 min. Then the NH₄Cl (50 mmol) was added and the resulting mixture was stirred over night. Water (50 ml) was added to the reaction mixture and extracted with EtOAc (2×100 mL). The resulting solution was concentrated in vacuo to yield a crude intermediate product amide 3.

The mixture of the intermediate amide 3, isobutyric anhydride (10 ml) and isobutyric acid (10 ml) was heated at 150° C. for 1 h under microwave. The resulting mixture was evaporated, dissolved in water, extracted with EtOAc, and purified by column chromatography to afford intermediate 4 (42% yield for steps).

After stirring the mixture of intermediate compound 4 (2.0 mmol), NCS (2.1 mmol) in NMP (5 mL) and H₂O (0.5 mL) at 80° C. for 15 min, amines (4.0 mmol) was added, stirred at 80° C. for 8 h, and purified by prep HPLC to give intermediate compound 5 (60% yield).

The mixture of intermediate compound 5 (0.2 mmol), Et₃N (0.4 mmol) in CH₂Cl₂ (1 mL) and DMF (0.1 mL) were further functionalized by the addition of alkyl acid chlorides, aryl acid chlorides, or isocyanates (0.2 mmol) and purified by prep HPLC to give compounds of general formulas 6 or 7 (62% yield).

### Example II-1

### 4-Isopropylamino-2-methylsulfanyl-pyrimidine-5-carboxylic acid ethyl ester

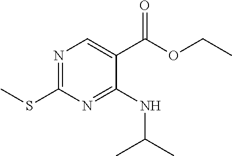 [SMILES: CCOC(=O)c1cnc(SC)nc1NC(C)C]

Procedures in Scheme 6 were utilized to synthesize this compound. ¹H NMR (CDCl₃, 400 MHz) δ 8.62 (s, 1H), 8.16 (d, J=5.4 Hz, 1H), 4.42-4.38 (m, 1H), 4.35-4.29 (m, 2H), 2.54 (s, 3H), 1.38 (t, J=7.1 Hz, 3H), 1.29 (q, J=6.5 Hz, 6H); LC/MS (M+H⁺) 256.

### Example II-2

### 8-Isopropyl-2-methylsulfanyl-8H-pyrido[2,3-d]pyrimidin-7-one

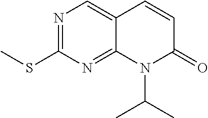 [SMILES: CSc1ncc2ccc(=O)n(C(C)C)c2n1]

Procedures in Scheme 6 were utilized to synthesize this compound. ¹H NMR (DMSO-d₆, 400 MHz) δ 8.86 (s, 1H), 7.88 (d, J=9.4 Hz, 1H), 6.57 (d, J=9.3 Hz, 1H), 5.69 (s, 1H), 2.60 (s, 3H), 1.55 (d, J=6.8 Hz, 6H); LC/MS (M+H⁺) 236.

### Example II-3

### 8-Cyclopentyl-2-methylsulfanyl-8H-pyrido[2,3-d]pyrimidin-7-one

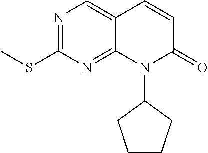 [SMILES: CSc1ncc2ccc(=O)n(C3CCCC3)c2n1]

¹H NMR (DMSO-d₆, 400 MHz) δ 8.85 (s, 1H), 7.86 (d, J=9.4 Hz, 1H), 6.56 (d, J=9.4 Hz, 1H), 5.84-5.78 (m, 1H), 2.57 (s, 3H), 2.24-2.18 (m, 2H), 1.99-1.94 (m, 2H), 1.80-1.79 (m, 2H), 1.64-1.60 (m, 2H); LC/MS (M+H⁺) 262.

### Example II-4

### Trans-2-(4-Amino-cyclohexylamino)-8-isopropyl-8H-pyrido[2,3-d]pyrimidin-7-one

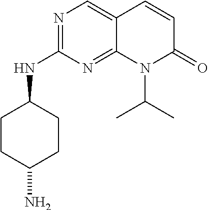 [SMILES: CC(C)n1c(=O)ccc2cnc(N[C@H]3CC[C@H](N)CC3)nc21]

Procedures in Scheme 6 were utilized to synthesize this compound. ¹H NMR (DMSO-d₆, 400 MHz) δ 8.56 (s, 1H), 7.84 (s, br, 3H), 7.66 (d, J=9.2 Hz, 1H), 6.23-6.16 (m, 1H), 5.65-5.62 (m, 1H), 3.79-3.68 (m, 1H), 3.04 (s, br, 1H), 2.02-1.95 (m, 4H), 1.54 (d, J=6.8 Hz, 3H), 1.53-1.40 (m, 4H); LC/MS (M+H⁺) 398.

### Example II-5

### Trans-2-((4-hydroxycyclohexyl)amino)-8-isopropylpyrido[2,3-d]pyrimidin-7 (8H)-one

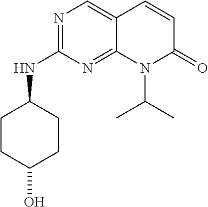 [SMILES: CC(C)n1c(=O)ccc2cnc(N[C@H]3CC[C@H](O)CC3)nc21]

Procedures in Scheme 6 were utilized to synthesize this compound. LC/MS (M+H⁺) 303.

### Example II-6

### 2-(1-Benzyl-piperidin-4-ylamino)-8-isopropyl-8H-pyrido[2,3-d]pyrimidin-7-one

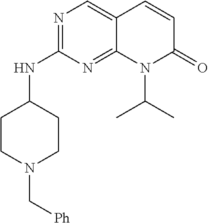 [SMILES: CC(C)n1c(=O)ccc2cnc(NC3CCN(Cc4ccccc4)CC3)nc21]

Procedures in Scheme 6 were utilized to synthesize this compound. 1H NMR (DMSO-d6, 400 MHz) δ 8.63-8.59 (m, 1H), 8.03 (d, J=7.3 Hz, 1H), 7.72-7.65 (m, 1H), 7.56-7.49 (m, 5H), 6.25-6.19 (m, 1H), 4.35-4.31 (m, 2H), 4.03-3.96 (m, 1H), 3.53-3.38 (m, 2H), 3.28-3.13 (m, 2H), 2.16-2.06 (m, 2H), 1.79-1.70 (m, 2H), 1.54-1.50 (m, 6H); LC/MS (M+H+) 378.

### Example II-7

### 2-(4-Hydroxymethyl-piperidin-1-yl)-8-isopropyl-8H-pyrido[2,3-d]pyrimidin-7-one

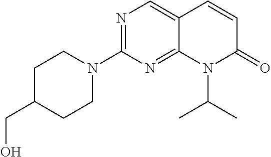 [SMILES: CC(C)n1c(=O)ccc2cnc(N3CCC(CO)CC3)nc21]

Procedures in Scheme 6 were utilized to synthesize this compound. ¹H NMR (DMSO-d₆, 400 MHz) δ 8.62 (s, 1H), 7.67 (d, J=9.2 Hz, 1H), 6.21 (d, J=9.2 Hz, 1H), 5.62 (s, br, 1H), 4.76-4.72 (m, 2H), 3.29-3.26 (m, 2H), 3.02-2.94 (m, 2H), 1.77-1.71 (m, 3H), 1.52 (d, J=6.8 Hz, 6H), 1.16-1.09 (m, 2H); LC/MS (M+H⁺) 303.

### Example II-8

### Trans-N-[4-(8-Isopropyl-7-oxo-7,8-dihydro-pyrido[2,3-d]pyrimidin-2-ylamino)-cyclohexyl]-isobutyramide

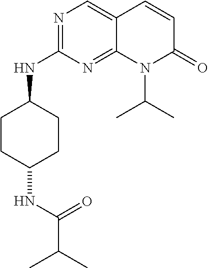 [SMILES: CC(C)C(=O)N[C@H]1CC[C@H](Nc2ncc3ccc(=O)n(C(C)C)c3n2)CC1]

Procedures in Scheme 6 were utilized to synthesize this compound. ¹H NMR (MeOD-d₄, 400 MHz) δ 8.54 (s, 1H), 7.66 (d, J=8.4 Hz, 1H), 6.31 (s, br, 1H), 5.77 (s, br, 1H), 3.89-3.86 (m, 1H), 3.69-3.66 (m, 1H), 2.43-2.39 (m, 1H), 2.16-2.10 (m, 2H), 2.00-1.96 (m, 2H), 1.60 (s, br, 6H), 1.59-1.38 (m, 4H), 1.10 (d, J=6.8 Hz, 6H); LC/MS (M+H⁺) 372.

### Example II-9

### Trans-N-4-((8-isopropyl-7-oxo-7,8-dihydropyrido[2,3-d]pyrimidin-2-yl)amino)cyclohexyl)methane-sulfonamide

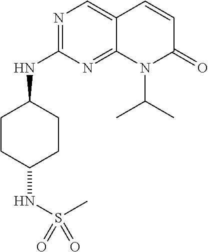 [SMILES: CC(C)n1c(=O)ccc2cnc(N[C@H]3CC[C@H](NS(C)(=O)=O)CC3)nc21]

Procedures in Scheme 6 were utilized to synthesize this compound. ¹H NMR (DMSO-d₆, 400 MHz) δ 8.57 (d, J=13.2 Hz, 1H), 7.72 (dd, J=7.2, 57.5 Hz, 1H), 7.65 (d, J=9.1 Hz, 1H), 7.02 (d, J=7.1 Hz, 1H), 6.21-6.15 (m, 1H), 5.67-5.53 (m, 1H), 3.78-3.58 (m, 1H), 3.12 (s, br, 1H), 2.92 (s, 3H), 2.04-1.87 (m, 4H), 1.54 (d, J=6.6 Hz, 6H), 1.42-1.28 (m, 4H); LC/MS (M+H⁺) 380.

### Example II-10

### Trans-1-Ethyl-3-[4-(8-isopropyl-7-oxo-7,8-dihydro-pyrido[2,3-d]pyrimidin-2-ylamino)-cyclohexyl]-urea

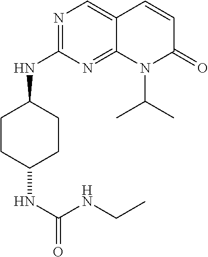 [SMILES: CCNC(=O)N[C@H]1CC[C@H](Nc2ncc3ccc(=O)n(C(C)C)c3n2)CC1]

Procedures in Scheme 6 were utilized to synthesize this compound. ¹H NMR (MeOD-d₄, 400 MHz) δ 8.53 (s, 1H), 7.66 (d, J=9.4 Hz, 1H), 6.33 (d, J=7.6 Hz, 1H), 5.76 (s, br, 1H), (m, 1H), 5.75 (s, br, 1H), 3.93-3.85 (m, 1H), 3.55-3.48 (m, 1H), 2.15-1.99 (m, 4H), 1.59 (s, br, 6H), 1.58-1.48 (m, 2H), 1.38-1.29 (m, 2H), 1.09 (t, J=7.2 Hz, 3H); LC/MS (M+H⁺) 373.

### Example II-11

### Trans-1-Isopropyl-3-[4-(8-isopropyl-7-oxo-7,8-dihydro-pyrido[2,3-d]pyrimidin-2-ylamino)-cyclohexyl]-urea

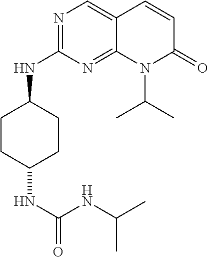 [SMILES: CC(C)NC(=O)N[C@H]1CC[C@H](Nc2ncc3ccc(=O)n(C(C)C)c3n2)CC1]

Procedures in Scheme 6 were utilized to synthesize this compound. ¹H NMR (MeOD-d₄, 400 MHz) δ 8.54 (s, 1H), 7.66 (d, J=9.4 Hz, 1H), 6.35-6.33 (m, 1H), 5.76 (s, br, 1H), 3.88-3.76 (m, 2H), 3.55-3.48 (m, 1H), 2.15-1.99 (m, 4H), 1.60 (s, br, 6H), 1.59-1.29 (m, 4H), 1.11 (d, J=6.6 Hz, 6H); LC/MS (M+H⁺) 387.

### Example II-12

### 2-((1-Isobutyrylpiperidin-4-yl)amino)-8-isopropylpyrido[2,3-d]pyrimidin-7(8H)-one

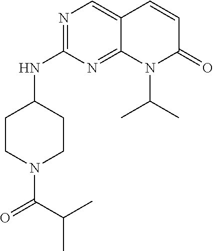 [SMILES: CC(C)C(=O)N1CCC(Nc2ncc3ccc(=O)n(C(C)C)c3n2)CC1]

Procedures in Scheme 6 were utilized to synthesize this compound. ¹H NMR (DMSO-d₆, 400 MHz) δ 8.59 (d, J=12.3 Hz, 1H), 7.76 (dd, J=7.2, 75.6 Hz, 1H), 7.66 (d, J=9.3 Hz, 1H), 6.22-6.16 (m, 1H), 5.65 (s, br, 1H), 4.39-4.28 (m, 1H), 4.13-3.91 (m, 2H), 2.94-2.86 (m, 1H), 2.83-2.64 (m, 1H), 2.01-1.82 (m, 2H), 1.58-1.34 (m, 4H), 1.02 (s, 6H); LC/MS (M+H⁺) 358.

### Example II-13

### 8-Isopropyl-2-((1-(methylsulfonyl)piperidin-4-yl)amino)pyrido[2,3-d]pyrimidin-7(8H)-one

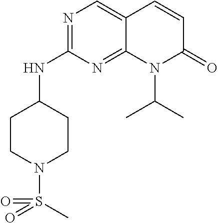 [SMILES: CC(C)n1c(=O)ccc2cnc(NC3CCN(S(C)(=O)=O)CC3)nc21]

Procedures in Scheme 6 were utilized to synthesize this compound. ¹H NMR (DMSO-d₆, 400 MHz) δ 8.62-8.56 (m, 1H), 7.83 (dd, J=7.2, 61.6 Hz, 1H), 7.67 (d, J=9.2 Hz, 1H), 6.25-6.16 (m, 1H), 5.75-5.61 (m, 1H), 4.04-3.85 (m, 1H), 3.59-3.52 (m, 2H), 2.90 (s, 3H), 2.02-1.93 (m, 2H), 1.68-1.58 (m, 2H), 1.55-1.45 (m, 6H); LC/MS (M+H⁺) 366.

### Example II-14

### N-ethyl-4-((8-isopropyl-7-oxo-7,8-dihydropyrido[2,3-d]pyrimidin-2-yl)amino)piperidine-1-carboxamide

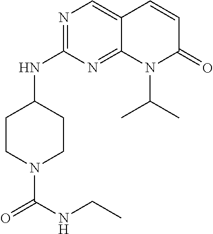 [SMILES: CCNC(=O)N1CCC(Nc2ncc3ccc(=O)n(C(C)C)c3n2)CC1]

Procedures in Scheme 6 were utilized to synthesize this compound. ¹H NMR (MeOD-d₄, 400 MHz) δ 8.55 (s, 1H), 7.67 (d, J=9.3 Hz, 1H), 6.34-6.30 (m, 1H), 5.78 (s, br, 1H), 4.06-4.01 (m, 3H), 3.19 (q, J=7.2 Hz, 2H), 3.02-2.96 (m, 2H), 2.04-1.98 (m, 2H), 1.62-1.44 (m, 8H), 1.11 (t, J=4.8 Hz, 3H); LC/MS (M+H⁺) 359.

### Example II-15

### Trans-[4-(8-Isopropyl-7-oxo-7,8-dihydro-pyrido[2,3-d]pyrimidin-2-ylamino)-cyclohexyl]-carbamic acid tert-butyl ester

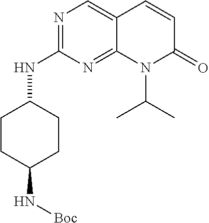

Procedures in Scheme 6 were utilized to synthesize this compound. ¹H NMR (DMSO-d₆, 400 MHz) δ 8.56 (d, J=13.6 Hz, 1H), 7.70 (dd, J=7.1, 37.8 Hz, 1H), 7.65-7.59 (m, 1H), 6.76 (d, J=7.7 Hz, 1H), 6.21-6.13 (m, 1H), 5.74-5.58 (m, 1H), 3.58-3.42 (m, 1H), 3.28-3.17 (m, 1H), 1.98-1.77 (m, 4H), 1.53 (d, J=6.4 Hz, 6H), 1.39 (s, 9H), 1.38-1.24 (m, 4H); LC/MS (M+H⁺) 402.

### Example II-16

### Cis-[4-(8-Isopropyl-7-oxo-7,8-dihydro-pyrido[2,3-d]pyrimidin-2-ylamino)-cyclohexyl]-carbamic acid tert-butyl ester

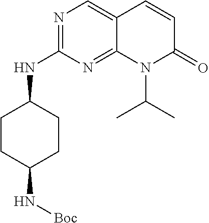 [SMILES: CC(C)n1c(=O)ccc2cnc(N[C@H]3CC[C@@H](NC(=O)OC(C)(C)C)CC3)nc21]

Procedures in Scheme 6 were utilized to synthesize this compound. ¹H NMR (DMSO-d₆, 400 MHz) δ 8.56 (d, J=13.6 Hz, 1H), 7.67-7.46 (m, 2H), 6.72 (s, br, 1H), 6.23-6.15 (m, 1H), 5.64 (s, br, 1H), 3.84 (s, br, 1H), 3.46 (s, br, 1H), 1.78-1.65 (m, 6H), 1.65-1.51 (m, 8H), 1.40 (s, 9H); LC/MS (M+H⁺) 402.

### Example II-17

### Cis-2-(4-Amino-cyclohexylamino)-8-isopropyl-8H-pyrido[2,3-d]pyrimidin-7-one

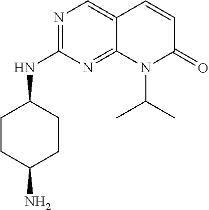 [SMILES: CC(C)n1c(=O)ccc2cnc(N[C@H]3CC[C@@H](N)CC3)nc21]

Procedures in Scheme 6 were utilized to synthesize this compound; LC/MS (M+H⁺) 302.

### Example II-18

### Cis-N-[4-(8-Isopropyl-7-oxo-7,8-dihydro-pyrido[2,3-d]pyrimidin-2-ylamino)-cyclohexyl]-isobutyramide

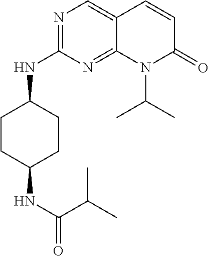 [SMILES: CC(C)C(=O)N[C@H]1CC[C@@H](Nc2ncc3ccc(=O)n(C(C)C)c3n2)CC1]

Procedures in Scheme 6 were utilized to synthesize this compound. ¹H NMR (DMSO-d₆, 400 MHz) δ 8.57 (s, 1H), 7.76-7.51 (m, 3H), 6.24-6.15 (m, 1H), 5.75-5.54 (m, 1H), 3.72-3.64 (m, 2H), 2.47-2.42 (m, 1H), 1.81-1.62 (m, 6H), 1.54-1.43 (m, 8H), 0.99 (d, J=6.8 Hz, 6H); LC/MS (M+H⁺) 372.

### Example II-19

### Cis-1-Ethyl-3-[4-(8-isopropyl-7-oxo-7,8-dihydro-pyrido[2,3-d]pyrimidin-2-ylamino)-cyclo-hexyl]-urea (SR-12427)

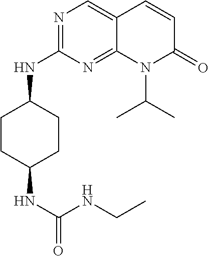 [SMILES: CCNC(=O)N[C@H]1CC[C@@H](Nc2ncc3ccc(=O)n(C(C)C)c3n2)CC1]

Procedures in Scheme 6 were utilized to synthesize this compound. ¹H NMR (MeOD-d₄, 400 MHz) δ 8.56 (s, 1H), 7.67 (d, J=9.4 Hz, 1H), 6.35 (d, J=8.8 Hz, 1H), 5.77 (s, br, 1H), 4.07-4.03 (m, 1H), 3.75-3.71 (m, 1H), 3.15 (q, J=7.2 Hz, 2H), 1.88-1.72 (m, 8H), 1.59 (d, J=6.8 Hz, 6H), 1.10 (t, J=7.2 Hz, 3H); LC/MS (M+H⁺) 373.

### Example II-20

### Trans-8-Cyclopentyl-2-(4-hydroxy-cyclohexylamino)-8H-pyrido[2,3-d]pyrimidin-7-one

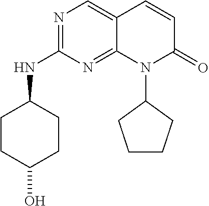 [SMILES: O=c1ccc2cnc(N[C@H]3CC[C@H](O)CC3)nc2n1C1CCCC1]

Procedures in Scheme 6 were utilized to synthesize this compound. ¹H NMR (DMSO-d₆, 400 MHz) δ 8.54-8.46 (m, 1H), 7.68-7.44 (m, 2H), 6.16-6.07 (m, 1H), 5.80-5.64 (m, 1H), 3.74-3.62 (m, 1H), 3.34 (s, br, 1H), 2.45-2.02 (m, 2H), 1.89-1.75 (m, 6H), 1.71-1.49 (m, 4H), 1.31-1.12 (m, 4H); LC/MS (M+H⁺) 329.

### Example II-21

### 4-(8-Cyclopentyl-7-oxo-7,8-dihydro-pyrido[2,3-d]pyrimidin-2-ylamino)-piperidine-1-carboxylic acid isopropylamide

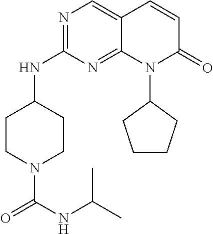 [SMILES: CC(C)NC(=O)N1CCC(Nc2ncc3ccc(=O)n(C4CCCC4)c3n2)CC1]

Procedures in Scheme 6 were utilized to synthesize this compound. ¹H NMR (MeOD-d₄, 400 MHz) δ 8.54 (s, 1H), 7.67 (d, J=9.3 Hz, 1H), 6.35-6.28 (m, 1H), 5.97-5.93 (m, 1H), 4.09-3.99 (m, 3H), 3.94-3.85 (m, 1H), 2.98-2.89 (m, 2H), 2.39 (s, br, 1H), 2.09-1.95 (m, 4H), 1.91-1.79 (m, 2H), 1.78-1.62 (m, 2H), 1.58-1.44 (m, 2H), 1.15 (d, J=6.6 Hz); LC/MS (M+H⁺) 399.

### Example II-22

### 4-(8-Isopropyl-7-oxo-7,8-dihydro-pyrido[2,3-d]pyrimidin-2-ylamino)-piperidine-1-carboxylic acid isopropylamide

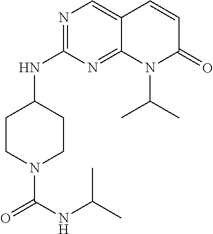 [SMILES: CC(C)NC(=O)N1CCC(Nc2ncc3ccc(=O)n(C(C)C)c3n2)CC1]

Procedures in Scheme 6 were utilized to synthesize this compound. ¹H NMR (MeOD-d₄, 400 MHz) δ 8.54 (s, 1H), 7.67 (d, J=9.4 Hz, 1H), 6.32 (d, J=7.7 Hz, 1H), 5.78 (s, br, 1H), 4.16-4.02 (m, 3H), 3.95-3.85 (m, 1H), 3.02-2.91 (m, 2H), 2.06-1.97 (m, 2H), 1.64-1.47 (m, 8H), 1.50 (d, J=6.6 Hz, 6H); LC/MS (M+H⁺) 373.

### Example II-23

### Trans-1-[4-(8-Cyclopentyl-7-oxo-7,8-dihydro-pyrido[2,3-d]pyrimidin-2-ylamino)-cyclohexyl]-3-isopropyl-urea

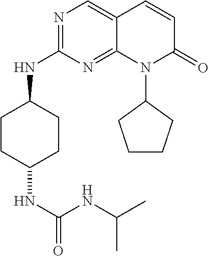 [SMILES: CC(C)NC(=O)N[C@H]1CC[C@H](Nc2ncc3ccc(=O)n(C4CCCC4)c3n2)CC1]

Procedures in Scheme 6 were utilized to synthesize this compound. ¹H NMR (MeOD-d₄, 400 MHz) δ 8.54 (s, 1H), 7.66 (d, J=9.3 Hz, 1H), 6.34-6.29 (m, 1H), 5.89 (s, br, 1H), 3.89-3.75 (m, 2H), 3.58-3.47 (m, 1H), 2.49-2.22 (m, 2H), 2.14-1.97 (m, 6H), 1.91-1.79 (m, 2H), 1.78-1.61 (m, 2H), 1.57-1.42 (m, 2H), 1.37-1.24 (m, 2H), 1.11 (d, J=6.5 Hz, 6H); LC/MS (M+H⁺) 413.

### Example II-24

### Trans-1-cyclopentyl-3-(4-((8-cyclopentyl-7-oxo-7,8-dihydropyrido[2,3-d]pyrimidin-2-yl)amino)cyclohexyl)urea

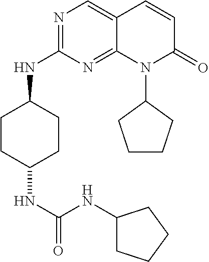 [SMILES: O=C(NC1CCCC1)N[C@H]1CC[C@H](Nc2ncc3ccc(=O)n(C4CCCC4)c3n2)CC1]

Procedures in Scheme 6 were utilized to synthesize this compound. ¹H NMR (400 MHz, DMSO) δ 8.59 (d, J=13.8 Hz, 1H), 7.67 (d, J=9.2 Hz, 1H), 6.23 (d, J=9.1 Hz, 2H), 5.78 (dd, J=31.9, 23.4 Hz, 2H), 3.99 (d, J=13.2 Hz, 3H), 3.89 (s, 2H), 2.71 (d, J=27.4 Hz, 2H), 2.35 (d, J=15.5 Hz, 1H), 2.18 (s, 1H), 1.95 (s, 2H), 1.90-1.69 (m, 6H), 1.67-1.55 (m, 3H), 1.55-1.31 (m, 6H). LC/MS (M+H⁺) 439.

### Example II-25

### Trans 2-((4-hydroxycyclohexyl)amino)pyrido[2,3-d]pyrimidin-7(8H)-one

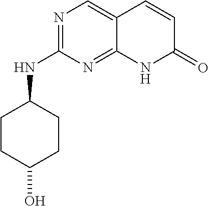 [SMILES: O=c1ccc2cnc(N[C@H]3CC[C@H](O)CC3)nc2[nH]1]

Procedures in Scheme 6 were utilized to synthesize this compound. LC/MS (M+H⁺) 261.

### Example II-26

### Trans-2-((4-aminocyclohexyl)amino)pyrido[2,3-d]pyrimidin-7(8H)-one

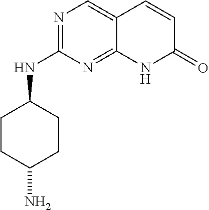 [SMILES: N[C@H]1CC[C@H](Nc2ncc3ccc(=O)[nH]c3n2)CC1]

Procedures in Scheme 6 were utilized to synthesize this compound. LC/MS (M+H⁺) 260.

### Example II-27

### Trans-1-isopropyl-3-((4-((7-oxo-7,8-dihydropyrido[2,3-d]pyrimidin-2-yl)amino)cyclohexyl)urea

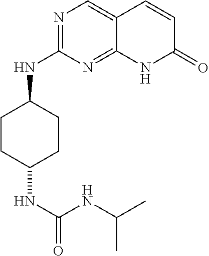 [SMILES: CC(C)NC(=O)N[C@H]1CC[C@H](Nc2ncc3ccc(=O)[nH]c3n2)CC1]

Procedures in Scheme 6 were utilized to synthesize this compound. LC/MS (M+H⁺) 345.

### Example II-28

### Trans-1-cyclopentyl-3-((4-((7-oxo-7, 8-dihydropyrido[2,3-d]pyrimidin-2-yl)amino)cyclohexyl)urea

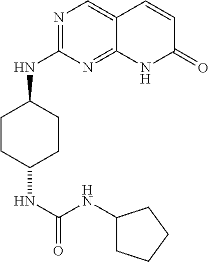 [SMILES: O=C(NC1CCCC1)N[C@H]1CC[C@H](Nc2ncc3ccc(=O)[nH]c3n2)CC1]

Procedures in Scheme 6 were utilized to synthesize this compound. LC/MS (M+H⁺) 371.

### Example II-29

### Trans 2-((4-hydroxycyclohexyl)amino)-8-methylpyrido[2,3-d]pyrimidin-7(8H)-one

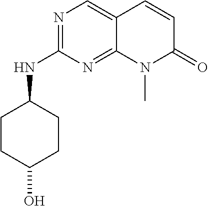 [SMILES: Cn1c(=O)ccc2cnc(N[C@H]3CC[C@H](O)CC3)nc21]

Procedures in Scheme 6 were utilized to synthesize this compound. ¹H NMR (400 MHz, DMSO) δ 8.52 (d, J=17.7 Hz, 1H), 7.64 (d, J=9.3 Hz, 1H), 6.18 (d, J=9.3 Hz, 1H), 3.67 (s, 1H), 3.51-3.35 (m, 3H), 3.29 (d, J=32.1 Hz, 1H), 1.87 (s, 2H), 1.80 (s, 3H), 1.37-1.08 (m, 4H). LC/MS (M+H⁺) 261.

### Example II-30

### Trans-2-((4-aminocyclohexyl)amino)-8-methylpyrido[2,3-d]pyrimidin-7(8H)-one

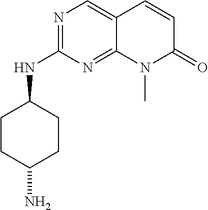 [SMILES: Cn1c(=O)ccc2cnc(N[C@H]3CC[C@H](N)CC3)nc21]

Procedures in Scheme 6 were utilized to synthesize this compound. LC/MS (M+H⁺) 274.

### Example II-31

### Trans-1-ethyl-3-((4-((8-methyl-7-oxo-7,8-dihydropyrido[2,3-d]pyrimidin-2-yl)amino)cyclohexyl)urea

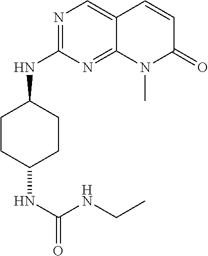 [SMILES: CCNC(=O)N[C@H]1CC[C@H](Nc2ncc3ccc(=O)n(C)c3n2)CC1]

Procedures in Scheme 6 were utilized to synthesize this compound. ¹H NMR (400 MHz, DMSO) δ 8.50 (s, 1H), 7.64 (d, J=9.3 Hz, 1H), 6.18 (d, J=9.3 Hz, 1H), 5.61 (s, 2H), 3.68 (s, 2H), 3.42 (d, J=16.7 Hz, 3H), 3.25 (s, 1H), 2.92 (d, J=7.2 Hz, 2H), 1.85 (d, J=26.0 Hz, 1H), 1.79 (s, 2H), 1.40-1.22 (m, 2H), 1.16 (m, 2H), 0.90 (t, J=7.2 Hz, 3H); LC/MS (M+H⁺) 345.

### Example II-32

### Trans-1-isopropyl-3-((4-((8-methyl-7-oxo-7,8-dihydropyrido[2,3-d]pyrimidin-2-yl)amino)cyclohexyl)urea

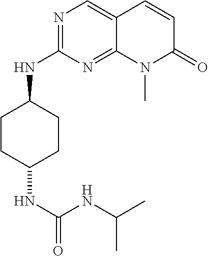 [SMILES: CC(C)NC(=O)N[C@H]1CC[C@H](Nc2ncc3ccc(=O)n(C)c3n2)CC1]

Procedures in Scheme 6 were utilized to synthesize this compound. ¹H NMR (400 MHz, DMSO) δ 8.59 (d, J=15.2 Hz, 1H), 7.71 (d, J=9.5 Hz, 1H), 6.25 (d, J=9.3 Hz, 1H), 5.57 (s, 2H), 3.66 (s, 3H), 3.47 (s, 3H), 3.33 (s, 1H), 1.96 (s, 1H), 1.86 (s, 2H), 1.38 (m, 2H), 1.20 (m, 9.5 Hz, 2H), 1.01 (d, J=6.5 Hz, 6H). LC/MS (M+H⁺) 3595.

### Example II-33

### Trans-1-cyclopentyl-3-((4-((8-methyl-7-oxo-7,8-dihydropyrido[2,3-d]pyrimidin-2-yl)amino)cyclohexyl)urea

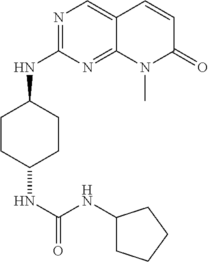 [SMILES: Cn1c(=O)ccc2cnc(N[C@H]3CC[C@H](NC(=O)NC4CCCC4)CC3)nc21]

Procedures in Scheme 6 were utilized to synthesize this compound. ¹H NMR (400 MHz, DMSO) δ 8.59 (d, J=13.9 Hz, 1H), 7.70 (t, J=9.3 Hz, 1H), 6.25 (d, J=9.3 Hz, 1H), 5.73 (s, 2H), 5.55 (s, 1H), 3.83 (dt, J=13.3, 6.6 Hz, 2H), 3.49 (d, J=16.7 Hz, 2H), 3.33 (s, 1H), 1.96 (s, 1H), 1.86 (s, 2H), 1.75 (dt, J=11.7, 6.0 Hz, 2H), 1.64-1.52 (m, 2H), 1.52-1.44 (m, 2H), 1.38 (dd, J=25.1, 13.2 Hz, 2H), 1.24 (ddd, J=33.4, 19.0, 9.0 Hz, 4H). LC/MS (M+H⁺) 385.

### Example II-34

### 8-methyl-2-(piperidin-4-ylamino)pyrido[2,3-d]pyrimidin-7(8H)-one

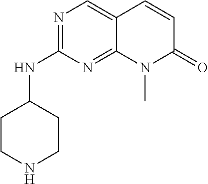 [SMILES: Cn1c(=O)ccc2cnc(NC3CCNCC3)nc21]

Procedures in Scheme 6 were utilized to synthesize this compound. LC/MS (M+H⁺) 260.

### Example II-35

### N-isopropyl-4-((8-methyl-7-oxo-7,8-dihydropyrido[2,3-d]pyrimidin-2-yl)amino)piperidine-1-carboxamide

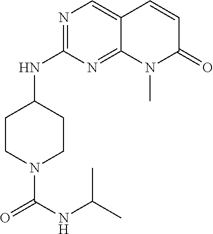 [SMILES: CC(C)NC(=O)N1CCC(Nc2ncc3ccc(=O)n(C)c3n2)CC1]

Procedures in Scheme 6 were utilized to synthesize this compound. ¹H NMR (400 MHz, d₆-DMSO) δ 8.61 (d, J=13.2 Hz, 1H), 7.72 (d, J=9.3 Hz, 1H), 6.26 (d, J=9.3 Hz, 1H), 6.18 (s, 1H), 3.96 (d, J=10.8 Hz, 3H), 3.75 (d, J=6.4 Hz, 1H), 3.50 (d, J=15.6 Hz, 3H), 2.85-2.61 (m, 2H), 1.83 (dd, J=28.2, 11.7 Hz, 2H), 1.38 (d, J=11.9 Hz, 2H), 1.04 (t, J=10.4 Hz, 6H).

LC/MS (M+H⁺) 345.

### Example II-36

### N-cyclopentyl-4-((8-methyl-7-oxo-7,8-dihydropyrido[2,3-d]pyrimidin-2-yl)amino)piperidine-1-carboxamide

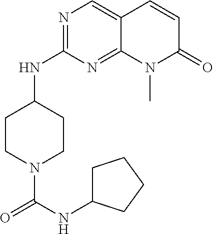 [SMILES: Cn1c(=O)ccc2cnc(NC3CCN(C(=O)NC4CCCC4)CC3)nc21]

Procedures in Scheme 6 were utilized to synthesize this compound. ¹H NMR (400 MHz, DMSO) δ 8.61 (d, J=13.7 Hz, 1H), 7.72 (d, J=9.3 Hz, 1H), 6.26 (d, J=9.3 Hz, 2H), 4.05-3.81 (m, 4H), 3.50 (d, J=15.4 Hz, 3H), 2.89-2.65 (m, 2H), 1.87 (d, J=10.8 Hz, 2H), 1.81-1.71 (m, 2H), 1.63 (t, J=7.5 Hz, 2H), 1.46 (td, J=7.6, 4.0 Hz, 2H), 1.43-1.31 (m, 4H). LC/MS (M+H⁺) 371.

### Example II-37

### Trans-4-((7-cyclopropylpyrido[2,3-d]pyrimidin-2-yl)amino)cyclohexanol

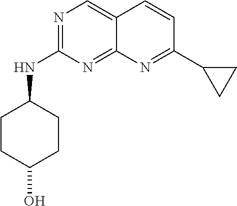 [SMILES: O[C@H]1CC[C@H](Nc2ncc3ccc(C4CC4)nc3n2)CC1]

Procedures in Scheme 7 were utilized to synthesize this compound. LC/MS (M+H⁺) 287.

### Example II-38

### Trans-N1-(7-cyclopropylpyrido[2,3-d]pyrimidin-2-yl)cyclohexane-1,4-diamine

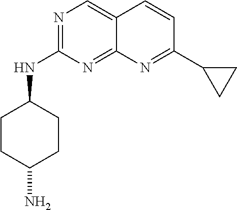 [SMILES: N[C@H]1CC[C@H](Nc2ncc3ccc(C4CC4)nc3n2)CC1]

Procedures in Scheme 7 were utilized to synthesize this compound. LC/MS (M+H⁺) 286.

### Example II-39

### Trans-1-ethyl-3-(4-((7-cyclopropylpyrido[2,3-d]pyrimidin-2-yl)amino)cyclohexyl)urea

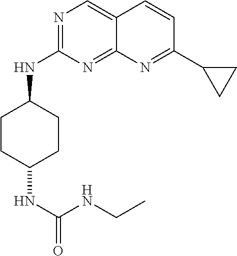 [SMILES: CCNC(=O)N[C@H]1CC[C@H](Nc2ncc3ccc(C4CC4)nc3n2)CC1]

Procedures in Scheme 7 were utilized to synthesize this compound. LC/MS (M+H⁺) 371.

### Example II-40

### 7-cyclopropyl-N-(piperidin-4-yl)pyrido[2,3-d]pyrimidin-2-amine

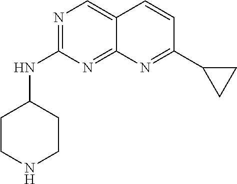 [SMILES: c1cc2cnc(NC3CCNCC3)nc2nc1C1CC1]

Procedures in Scheme 7 were utilized to synthesize this compound. LC/MS (M+H⁺) 272.

### Example II-41

### 1-(4-((7-cyclopropylpyrido[2,3-d]pyrimidin-2-yl)amino)piperidin-1-yl)-2-methylpropan-1-one

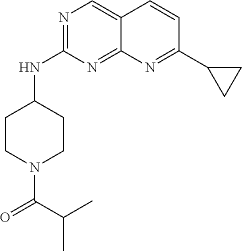 [SMILES: CC(C)C(=O)N1CCC(Nc2ncc3ccc(C4CC4)nc3n2)CC1]

Procedures in Scheme 7 were utilized to synthesize this compound. LC/MS (M+H⁺) 342.

### Example II-42

### 4-((7-cyclopropylpyrido[2,3-d]pyrimidin-2-yl)amino)-N-ethylpiperidine-1-carboxamide

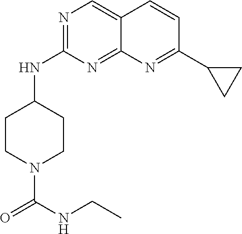 [SMILES: CCNC(=O)N1CCC(Nc2ncc3ccc(C4CC4)nc3n2)CC1]

Procedures in Scheme 7 were utilized to synthesize this compound. LC/MS (M+H⁺) 341.

### Example II-43

### Trans-4-((7-isopropylpyrido[2,3-d]pyrimidin-2-yl)amino)cyclohexanol

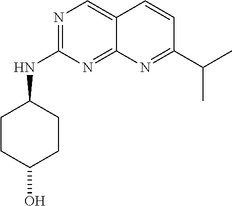 [SMILES: CC(C)c1ccc2cnc(N[C@H]3CC[C@H](O)CC3)nc2n1]

Procedures in Scheme 7 were utilized to synthesize this compound. LC/MS (M+H⁺) 313.

### Example II-44

### Trans-N1-(7-isopropylpyrido[2,3-d]pyrimidin-2-yl)cyclohexane-1,4-diamine

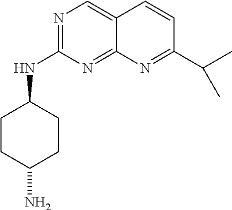 [SMILES: CC(C)c1ccc2cnc(N[C@H]3CC[C@H](N)CC3)nc2n1]

Procedures in Scheme 7 were utilized to synthesize this compound. LC/MS (M+H⁺) 312.

### Example II-45

### Trans-1-(4-((7-isopropylpyrido[2,3-d]pyrimidin-2-yl)amino)cyclohexyl)-3-isopropylurea

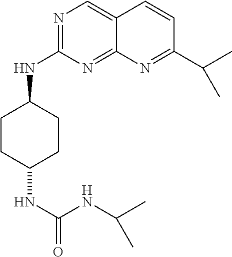 [SMILES: CC(C)NC(=O)N[C@H]1CC[C@H](Nc2ncc3ccc(C(C)C)nc3n2)CC1]

Procedures in Scheme 7 were utilized to synthesize this compound. LC/MS (M+H⁺) 397.

### Example II-46

### Trans-4-((7-(pyrrolidin-1-yl)pyrido[2,3-d]pyrimidin-2-yl)amino)cyclohexanol

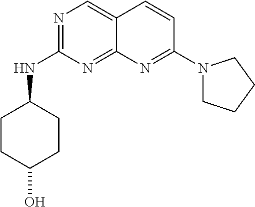 [SMILES: O[C@H]1CC[C@H](Nc2ncc3ccc(N4CCCC4)nc3n2)CC1]

Procedures in Scheme 8 were utilized to synthesize this compound. LC/MS (M+H⁺) 314.

### Example II-47

### Trans-N1-(7-(pyrrolidin-1-yl)pyrido[2,3-d]pyrimidin-2-yl)cyclohexane-1,4-diamine

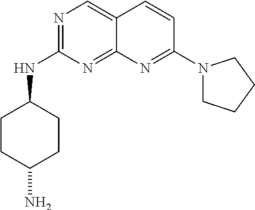 [SMILES: N[C@H]1CC[C@H](Nc2ncc3ccc(N4CCCC4)nc3n2)CC1]

Procedures in Scheme 8 were utilized to synthesize this compound. LC/MS (M+H⁺) 313.

### Example II-48

### Trans-1-isopropyl-3-(4-((7-(pyrrolidin-1-yl)pyrido[2,3-d]pyrimidin-2-yl)amino)cyclohexyl)urea

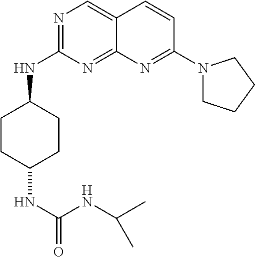 [SMILES: CC(C)NC(=O)N[C@H]1CC[C@H](Nc2ncc3ccc(N4CCCC4)nc3n2)CC1]

Procedures in Scheme 8 were utilized to synthesize this compound. LC/MS (M+H⁺) 398.

### Example II-49

### N-(piperidin-4-yl)-7-(pyrrolidin-1-yl)pyrido[2,3-d]pyrimidin-2-amine

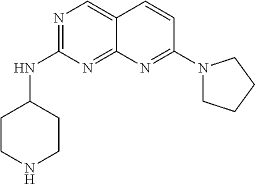 [SMILES: c1cc2cnc(NC3CCNCC3)nc2nc1N1CCCC1]

Procedures in Scheme 8 were utilized to synthesize this compound. LC/MS (M+H⁺) 299.

### Example II-50

### Trans-7-((4-hydroxycyclohexyl)amino)-2-isopropylpyrimido[4,5-d]pyrimidin-4(3H)-one

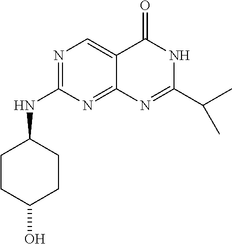 [SMILES: CC(C)c1nc2nc(N[C@H]3CC[C@H](O)CC3)ncc2c(=O)[nH]1]

Procedures in Scheme 9 were utilized to synthesize this compound. LC/MS (M+H⁺) 304.

### Example II-51

### Trans-7-((4-aminocyclohexyl)amino)-2-isopropylpyrimido[4,5-d]pyrimidin-4(3H)-one

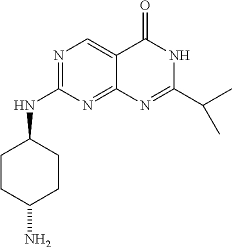 [SMILES: CC(C)c1nc2nc(N[C@H]3CC[C@H](N)CC3)ncc2c(=O)[nH]1]

Procedures in Scheme 9 were utilized to synthesize this compound. LC/MS (M+H⁺) 303.

### Example II-52

### Trans-1-isopropyl-3-(4-((7-isopropyl-5-oxo-5,6-dihydropyrimido[4,5-d]pyrimidin-2-yl)amino)cyclohexyl)urea

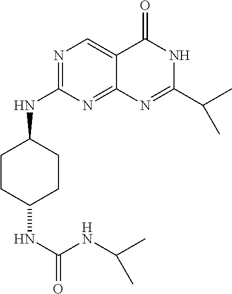 [SMILES: CC(C)NC(=O)N[C@H]1CC[C@H](Nc2ncc3c(=O)[nH]c(C(C)C)nc3n2)CC1]

Procedures in Scheme 9 were utilized to synthesize this compound. LC/MS (M+H⁺) 388.

### Example II-53

### N-((1r,4r)-4-((8-isopropyl-5-methyl-7-oxo-7,8-dihydropyrido[2,3-d]pyrimidin-2-yl)amino)cyclohexyl)isobutyramide

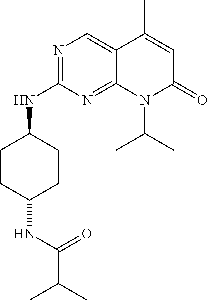 [SMILES: Cc1cc(=O)n(C(C)C)c2nc(N[C@H]3CC[C@H](NC(=O)C(C)C)CC3)ncc12]

### Example II-54

### N-((1r,4r)-4-((8-isopropyl-5,6-dimethyl-7-oxo-7,8-dihydropyrido[2,3-d]pyrimidin-2-yl)amino)cyclohexyl)isobutyramide

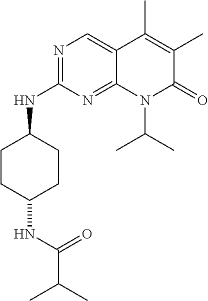 [SMILES: Cc1c(C)c2cnc(N[C@H]3CC[C@H](NC(=O)C(C)C)CC3)nc2n(C(C)C)c1=O]

### Example II-55

### N-((1r,4r)-4-(8-isopropyl-6-methyl-7-oxo-7,8-dihydropyrido[2,3-d]pyrimidin-2-yl)amino)cyclohexyl)isobutyramide

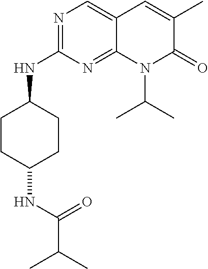 [SMILES: Cc1cc2cnc(N[C@H]3CC[C@H](NC(=O)C(C)C)CC3)nc2n(C(C)C)c1=O]

### Example II-56

### N-isopropyl-4-((8-isopropyl-5-methyl-7-oxo-7,8-dihydropyrido[2,3-d]pyrimidin-2-yl)amino)piperidine-1-carboxamide

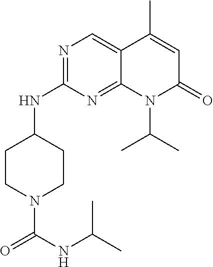 [SMILES: Cc1cc(=O)n(C(C)C)c2nc(NC3CCN(C(=O)NC(C)C)CC3)ncc12]

### Example II-57

### N-isopropyl-4-((8-isopropyl-5,6-dimethyl-7-oxo-7,8-dihydropyrido[2,3-d]pyrimidin-2-yl)amino)piperidine-1-carboxamide

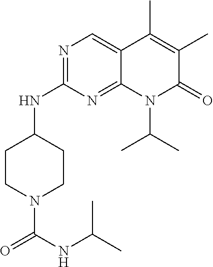 [SMILES: Cc1c(C)c2cnc(NC3CCN(C(=O)NC(C)C)CC3)nc2n(C(C)C)c1=O]

### Example II-58

### N-isopropyl-4-((8-isopropyl-6-methyl-7-oxo-7,8-dihydropyrido[2,3-d]pyrimidin-2-yl)amino)piperidine-1-carboxamide

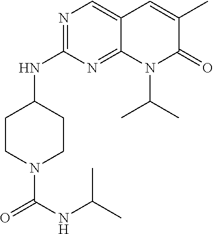 [SMILES: Cc1cc2cnc(NC3CCN(C(=O)NC(C)C)CC3)nc2n(C(C)C)c1=O]

### Biological Assays

### Homogeneous Time Resolved Fluorescence Assay—

Enzyme inhibition studies were performed in 384-well polystyrene HTRF plates (Grainier) for 22 min at ambient temperature (˜22° C.) with 0.2 μM biotinylated FL-ATF2, 1 μM ATP, 0.75 nM activated JNK3α1 (with a control in the absence of kinase for determining the basal signal) in 10 μL volumes containing the final concentrations of the following: 50 mM Hepes, pH 7.0, 2.5 mM MgCl₂, 0.1 mg/ml bovine serum albumin, 1 mM DL-dithiothreitol, 0.01% Triton X-100 (all from Sigma-Aldrich), and 5% DMSO (with or without compound). A 10 point titration of all compounds was carried out in 3-fold dilutions from 10 pM-2000 nM. After 22 min, the kinase reaction was terminated by addition of 10 μl of quenching solution [50 mM Hepes, pH 7.0, with 14 mM EDTA, 0.01% Triton X-100, 400 mM KF (all from Sigma-Aldrich)]. The detection reagents, streptavidin-x1APC (200 nM) and europium cryptate-labeled rabbit polyclonal anti-phospho-ATF2 (1 nM), were from Cis-Bio. The HTRF signal was detected using an Envision plate reader (Perkin Elmer) 1 h post-quenching. The data from multiple different experiments were averaged and presented as the mean±standard deviation. IC₅₀ values were determined by fitting the data to the equation for a four-parameter logistic. p38 enzyme inhibition assays were performed identically to the JNK3 assays with the exception that the reaction time was 22 min, 0.5 μM biotinylated FL-ATF2, ATP=11 μM, and p38 (Millipore)=3 nM.

### In-Cell Western Cell Based Assay:

SHSY5Y cells were plated at 75,000 cells/well in a 96-well clear bottom Packard View black Plate (Perkin Elmer) in DMEM:F12+10% FBS. Cells were allowed to attach overnight. The cells were then serum starved in 100 μl/well of 2% FBS DMEM:F12 for 24 hr in the incubator. Cells were treated with Compound (final concentrations ranged from 0.5 nM to 10 μM) in 0.01% DMSO final concentration for 1 h at 37° C. SHSY5Y cells were stressed activated for 4 hr with 35 μM 6-hydroxydopamine (6-OHDA). Following treatment, cells were immediately fixed with 4% paraformaldehyde for 30 minutes. After a brief wash in 0.1 M glycine, cells were permeabilized in 0.2% Triton X for 20 minutes. Cells were then washed once in PBS and blocked in LI-COR blocking buffer (LI-COR Biosciences) for 1 h at 25° C. Cells were probed for phosphorylated c-jun Ser 63 (Cell Signaling #9261) diluted 1:100 in Licor blocking buffer overnight at 4° C. Following three washes, cells were probed with goat anti-rabbit IR800 1:500 dilution in Licor blocking buffer+Tween-20 for 1 h at 25° C. Samples were washed 2× with PBST for 5 min each at RT, and then 1× with Licor blocking buffer+Tween-20. Nuclei were stained with TO-PRO-3 iodide (642/661) (1:4000) for 30 minutes at RT, washed twice in PBS/0.05% Tween-20 and read with an Odyssey Infrared Imaging System (LI-COR Biosciences).

All patents and publications referred to herein are incorporated by reference herein to the same extent as if each individual publication was specifically and individually indicated to be incorporated by reference in its entirety.

1. A compound of formula (I) 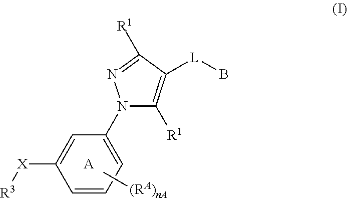
wherein
R¹ is independently at each occurrence H, CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′);
wherein any alkoxy, alkyl or cycloalkyl of R¹ is substituted with 0-3 (C₁-C₆)alkyl, (C₁-C₆)alkoxy, CN, CF₃, or halo;
ring A comprises 0-2 nitrogen atoms therein, provided that R³—X, the pyrazole bearing R¹, and any R^{A}, is bonded to a carbon atom of ring A; wherein R^{A} is independently at each occurrence CN, CF₃, halo, (C₁-C₆)alkyl, (C₁-C₆)alkoxy, (C₃-C₉)cycloalkyl, or (C₃-C₉)cycloalkoxy, wherein there are 0, 1, or 2 replacements of a respective methylene carbon atom of the alkyl, alkoxy, cycloalkyl, or cycloalkoxy by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′);
wherein any alkyl, alkoxy, cycloalkyl, or cycloalkoxy of R^{A} is substituted with 0-3 (C₁-C₆)alkoxy, CN, CF₃, or halo; and nA is 0, 1, 2, or 3, provided that nA is not greater than the number of carbon atoms in ring A minus two;
linker L is a bond, (CR′₂)ₙO(CR′₂)ₙ, (CR′₂)ₙ(N(R²))ₘ(CR′₂)ₙ, (CR′₂)ₙC(═O)(N(R²))ₘ(CR′₂)ₙ, (CR′₂)ₙ(N(R²))ₘC(═O)(N(R²))ₘ(CR′₂)ₙ, (CR′₂)ₙ(N(R′))ₘC(═S)(N(R′))ₘ(CR′₂)ₙ, (CR′₂)ₙOC(═O)(N(R²))ₘ(CR′₂)ₙ, (CR′₂)ₙSO₂(N(R²))ₘ(CR′₂)ₙ, or (CR′₂)ₙS(O)_{q}(CR′₂)ₙ,
wherein
m is independently at each occurrence 1 or 2,
n independently at each occurrence is 0, 1, 2, or 3, and
q=0, 1, or 2;
R′ is independently at each occurrence selected from the group consisting of H, (C₁-C₆)alkyl, and (C₁-C₆)acyl,
wherein any alkyl or acyl of R′ is substituted with 0, 1, or 2 independently selected R₂N or OR groups;
R is H or (C₁-C₆)alkyl, wherein alkyl is substituted with 0-3 (C₁-C₆)alkyl, (C₁-C₆)alkoxy, hydroxyl, NH₂, mono- or dialkylamino, CN, CF₃, or halo
R² is independently at each occurrence H, (C₁-C₆)alkyl, (C₁-C₆)acyl, or (C₃-C₉)cycloalkyl wherein there are 0, 1, or 2 replacements of a respective methylene carbon atom of the alkyl, acyl, or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′);
wherein any alkyl or cycloalkyl of R² is substituted with 0-3 (C₁-C₆)alkoxy, OH, R₂N, CN, CF₃, or halo; and,
B is (C₁-C₆)alkyl, (C₂-C₆)alkenyl, (C₂-C₆)alkynyl, mono- or bicyclic (C₆-C₁₀)aryl, mono- or bicyclic (C₆-C₁₀)aryloxy, mono- or bicyclic (C₆-C₁₀)aryl(C₁-C₆)alkyl, mono- or bicyclic (C₆-C₁₀) aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of B is substituted with 0-3 R^{B};
wherein R^{B} is independently at each occurrence CN, CF₃, halo, (C₁-C₆)alkoxy, (C₁-C₆)alkyl, or (C₃-C₉)cycloalkyl, wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O)O, SO₂N(R′), S(O), S(O)₂, C(═O)N(R′)N(R′), or N(R′)C(═O)N(R′); wherein any alkoxy, alkyl or cycloalkyl of R^{B} is substituted with 0-3 (C₁-C₆)alkoxy, CN, CF₃, or halo; or,
B and R², together with the nitrogen atom to which they are bonded, together form a 3-10 membered mono- or bicyclic heterocyclyl or heteroaryl, substituted with 0-3 (C₁-C₆)alkoxy, CN, CF₃, or halo;
X is a bond, (CR′₂)ₙO(CR′₂)ₙ, (CR′₂)ₙ(N(R′))ₘ(CR′₂)ₙ, (CR′₂)ₙC(═O)(N(R′))ₘ(CR′₂)ₙ, (CR′₂)ₙ(N(R′))ₘC(═O)(N(R′))m(CR′₂)ₙ, (CR′₂)ₙ(N(R′))ₘC(═S)(N(R′))ₘ(CR′₂)ₙ, (CR′₂)ₙOC(═O)(N(R′))ₘ(CR′₂)ₙ, (CR′₂)nSO₂(N(R′))ₘ(CR′₂)ₙ, or (CR′₂)ₙS(O)_{q}(CR′₂)ₙ,
wherein m is independently at each occurrence 1 or 2, n is independently at each occurrence is 0, 1, 2, or 3, and q=0, 1, or 2;
R³ is H, (C₁-C₆)alkyl or (C₃-C₉)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′),
wherein any alkyl or cycloalkyl is substituted with 0-3 (C₁-C₆)alkoxy, OH, R₂N, CN, CF₃, or halo; or R³ is (C₆-C₁₀) mono- or bicyclic aryl, or 3-10 membered mono- or bicyclic heteroaryl, wherein any aryl or heteroaryl of R³ is substituted with 0-3 R⁴; provided that if X is a bond, R³ is not H;
R⁴ is OH, R₂N, CN, CF₃, halo, (C₁-C₆)alkoxy, (C₁-C₆)alkyl or (C₃-C₉)cycloalkyl wherein there are 0, 1, or 2 replacements of a respective methylene carbon atom of the alkoxy, alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′);
wherein any alkoxy, alkyl or cycloalkyl of R⁴ is substituted with 0-3 (C₁-C₆)alkoxy, OH, R₂N, CN, CF₃, or halo, or R⁴ is mono- or bicyclic (C₆-C₁₀) aryl, mono- or bicyclic (C₆-C₁₀)aryloxy, mono- or bicyclic (C₆-C₁₀)aryl(C₁-C₆)alkyl, mono- or bicyclic (C₆-C₁₀) aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, heteroaryl, or heterocyclyl of R⁴ is substituted with 0-3 (C₁-C₆)alkoxy, (C₁-C₆)alkyl, CN, CF₃, or halo;
or a salt thereof.

2. The compound of claim 1, having formula (IA) 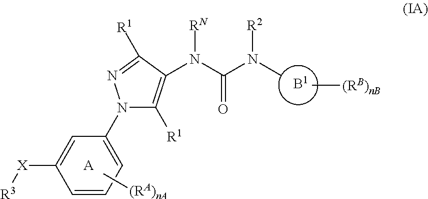
wherein
ring A, R, R′, R¹, R³, R⁴, R^{A}, nA, X, and R³ are as defined in claim 1, and wherein:
R² is H, (C₁-C₆)alkyl or (C₃-C₉)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′);
wherein any alkyl or cycloalkyl of R² is substituted with 0-3 (C₁-C₆)alkoxy, OH, R₂N, CN, CF₃, or halo;
R^{N} is H, or is (C₁-C₆)alkyl or (C₃-C₉)cycloalkyl wherein there are 0, 1, or 2 replacements of a respective methylene carbon atom of the alkyl or cycloalkyl by an independently selected N(R′), S, O, C(═S), C(═O), OC(═O), C(═O)C(═O), C(═O)N(R′), N(R′)C(═O), N(R′)C(═O)O, SO₂N(R′), N(R′)SO₂, S(O), S(O)₂, C(═O)N(R′)N(R′), or N(R′)C(═O)N(R′);
wherein any alkyl or cycloalkyl of R² is substituted with 0-3 (C1-C6)alkoxy, OH, R₂N, CN, CF₃, or halo;
B¹ is mono- or bicyclic (C₆-C₁₀)aryl, mono- or bicyclic (C₆-C₁₀)aryloxy, mono- or bicyclic (C₆-C₁₀)aryl(C₁-C₆)alkyl, mono- or bicyclic (C₆-C₁₀)aryl(C₁-C₆)alkoxy, mono- or bicyclic 3-10 membered heteroaryl, mono- or bicyclic 3-10 membered heteroaryloxy, mono- or bicyclic 3-10 membered heteroaryl(C₁-C₆)alkyl, mono- or bicyclic 3-10 membered heteroaryl(C₁-C₆)alkoxy, mono- or bicyclic 3-10 membered heterocyclyl, mono- or bicyclic 3-10 membered heterocycloxy, mono- or bicyclic 3-10 membered heterocyclyl(C₁-C₆)alkyl, or mono- or bicyclic 3-10 membered heterocyclyl(C₁-C₆)alkoxy, wherein any aryl, aryloxy, heteroaryl, heteroaryloxy, heterocyclyl, or heterocycloxy, is substituted with nB R^{B} groups; nB is 0, 1, 2, or 3, and R^{B} is independently at each occurrence as defined in claim 1; or,
B¹ and R², together with the nitrogen atom to which they are bonded, together form a 3-10 membered mono- or bicyclic heterocyclyl or heteroaryl, substituted with 0-3 (C₁-C₆)alkoxy, CN, CF₃, or halo;
or a salt thereof.

3. The compound of claim 1 wherein X is C(═O)NR′.

4. The compound of claim 1 wherein B is substituted phenyl.

5. The compound of claim 2 wherein B¹ is substituted phenyl.

6. The compound of claim 1 wherein R³ is substituted or unsubstituted heteroaryl or heterocyclyl.

7. The compound of claim 1 wherein R⁴ is heterocyclyl or heterocyclylalkyl.

8. The compound of claim 2 wherein B¹ and R², together with the nitrogen atom to which they are bonded, together form a 3-10 membered mono- or bicyclic heterocyclyl or heteroaryl, substituted with 0-3 (C1-C6)alkoxy, CN, CF₃, or halo.

9. The compound of claim 1 wherein ring A comprises 0 nitrogen atoms.

10. The compound of claim 1, wherein the compound is any one of 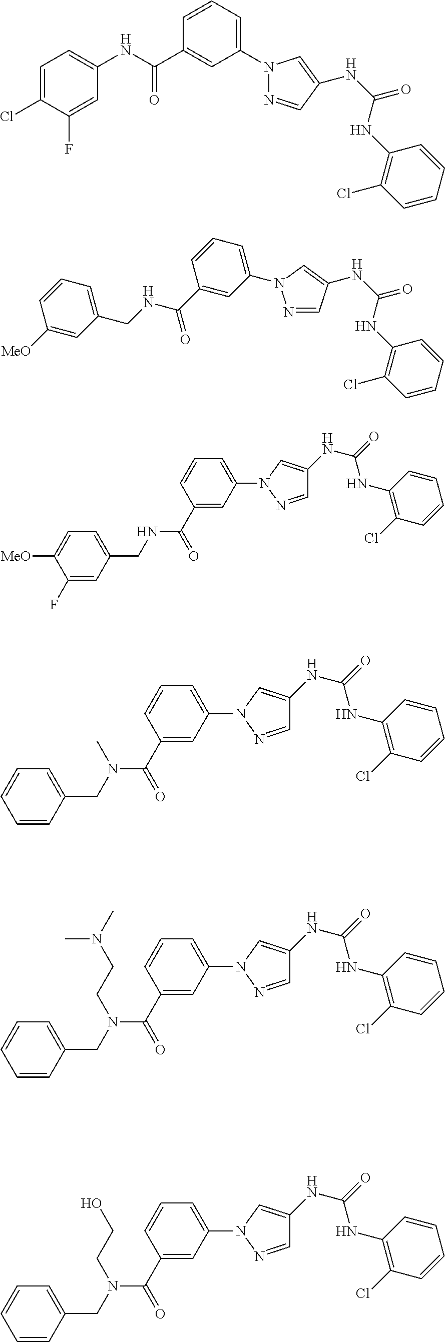 [SMILES: O=C(Nc1cnn(-c2cccc(C(=O)Nc3ccc(Cl)c(F)c3)c2)c1)Nc1ccccc1Cl ; COc1cccc(CNC(=O)c2cccc(-n3cc(NC(=O)Nc4ccccc4Cl)cn3)c2)c1 ; COc1ccc(CNC(=O)c2cccc(-n3cc(NC(=O)Nc4ccccc4Cl)cn3)c2)cc1F ; CN(Cc1ccccc1)C(=O)c1cccc(-n2cc(NC(=O)Nc3ccccc3Cl)cn2)c1 ; CN(C)CCN(Cc1ccccc1)C(=O)c1cccc(-n2cc(NC(=O)Nc3ccccc3Cl)cn2)c1 ; O=C(Nc1cnn(-c2cccc(C(=O)N(CCO)Cc3ccccc3)c2)c1)Nc1ccccc1Cl] 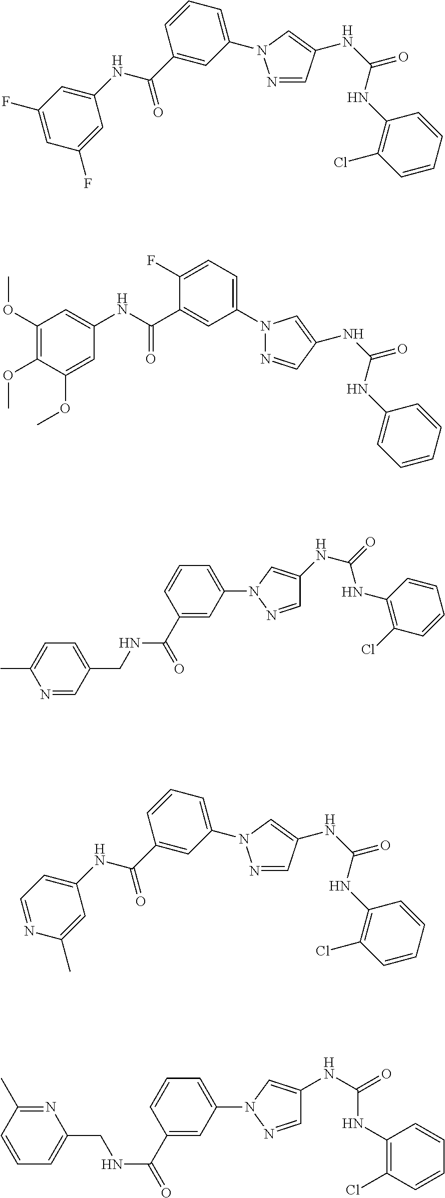 [SMILES: O=C(Nc1cnn(-c2cccc(C(=O)Nc3cc(F)cc(F)c3)c2)c1)Nc1ccccc1Cl ; COc1cc(NC(=O)c2cc(-n3cc(NC(=O)Nc4ccccc4)cn3)ccc2F)cc(OC)c1OC ; Cc1ccc(CNC(=O)c2cccc(-n3cc(NC(=O)Nc4ccccc4Cl)cn3)c2)cn1 ; Cc1cc(NC(=O)c2cccc(-n3cc(NC(=O)Nc4ccccc4Cl)cn3)c2)ccn1 ; Cc1cccc(CNC(=O)c2cccc(-n3cc(NC(=O)Nc4ccccc4Cl)cn3)c2)n1] 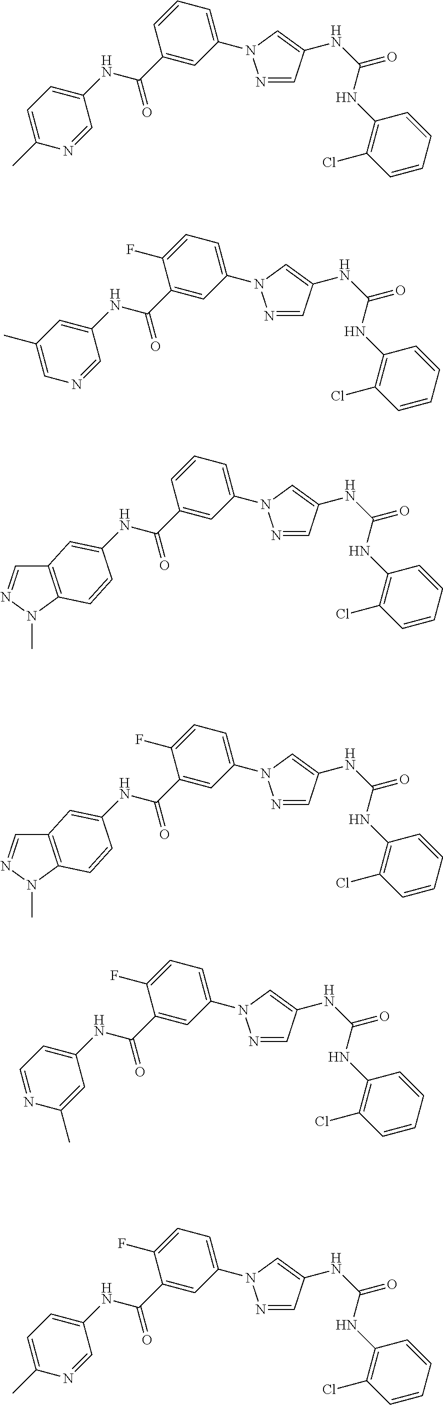 [SMILES: Cc1ccc(NC(=O)c2cccc(-n3cc(NC(=O)Nc4ccccc4Cl)cn3)c2)cn1 ; Cc1cncc(NC(=O)c2cc(-n3cc(NC(=O)Nc4ccccc4Cl)cn3)ccc2F)c1 ; Cn1ncc2cc(NC(=O)c3cccc(-n4cc(NC(=O)Nc5ccccc5Cl)cn4)c3)ccc21 ; Cn1ncc2cc(NC(=O)c3cc(-n4cc(NC(=O)Nc5ccccc5Cl)cn4)ccc3F)ccc21 ; Cc1cc(NC(=O)c2cc(-n3cc(NC(=O)Nc4ccccc4Cl)cn3)ccc2F)ccn1 ; Cc1ccc(NC(=O)c2cc(-n3cc(NC(=O)Nc4ccccc4Cl)cn3)ccc2F)cn1] 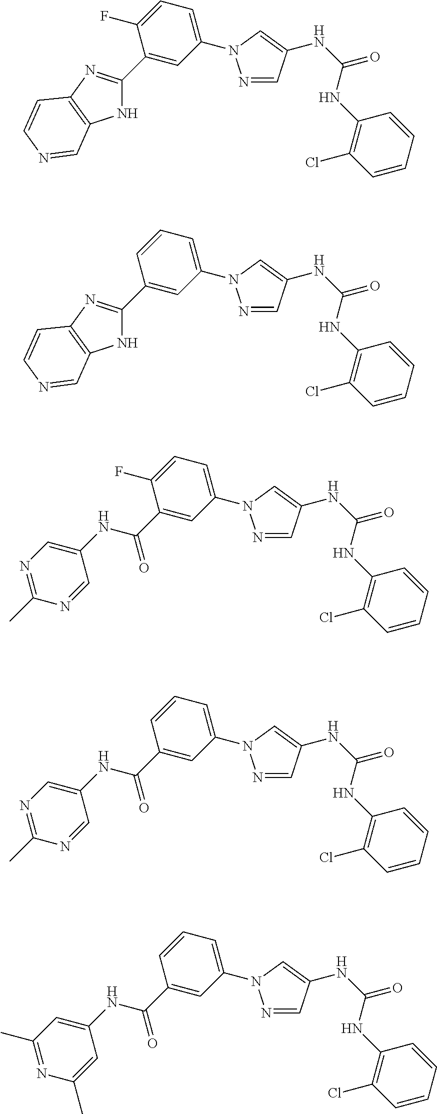 [SMILES: O=C(Nc1cnn(-c2ccc(F)c(-c3nc4ccncc4[nH]3)c2)c1)Nc1ccccc1Cl ; O=C(Nc1cnn(-c2cccc(-c3nc4ccncc4[nH]3)c2)c1)Nc1ccccc1Cl ; Cc1ncc(NC(=O)c2cc(-n3cc(NC(=O)Nc4ccccc4Cl)cn3)ccc2F)cn1 ; Cc1ncc(NC(=O)c2cccc(-n3cc(NC(=O)Nc4ccccc4Cl)cn3)c2)cn1 ; Cc1cc(NC(=O)c2cccc(-n3cc(NC(=O)Nc4ccccc4Cl)cn3)c2)cc(C)n1] 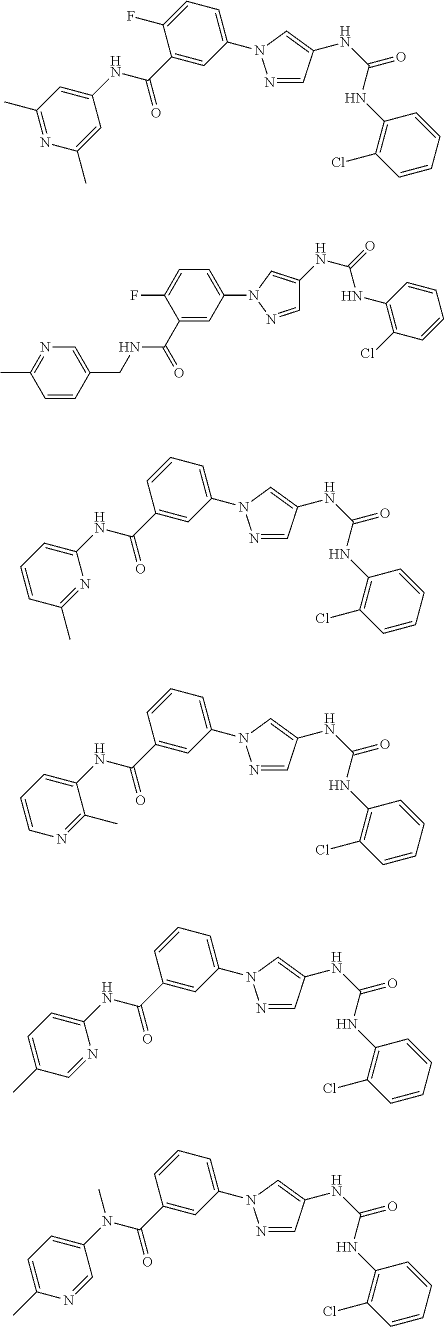 [SMILES: Cc1cc(NC(=O)c2cc(-n3cc(NC(=O)Nc4ccccc4Cl)cn3)ccc2F)cc(C)n1 ; Cc1ccc(CNC(=O)c2cc(-n3cc(NC(=O)Nc4ccccc4Cl)cn3)ccc2F)cn1 ; Cc1cccc(NC(=O)c2cccc(-n3cc(NC(=O)Nc4ccccc4Cl)cn3)c2)n1 ; Cc1ncccc1NC(=O)c1cccc(-n2cc(NC(=O)Nc3ccccc3Cl)cn2)c1 ; Cc1ccc(NC(=O)c2cccc(-n3cc(NC(=O)Nc4ccccc4Cl)cn3)c2)nc1 ; Cc1ccc(N(C)C(=O)c2cccc(-n3cc(NC(=O)Nc4ccccc4Cl)cn3)c2)cn1] 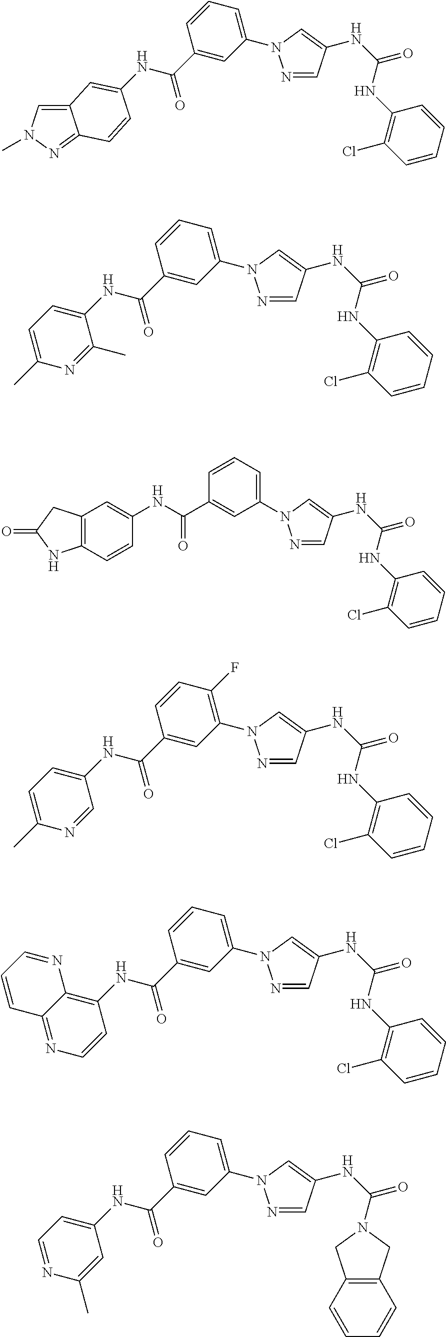 [SMILES: Cn1cc2cc(NC(=O)c3cccc(-n4cc(NC(=O)Nc5ccccc5Cl)cn4)c3)ccc2n1 ; Cc1ccc(NC(=O)c2cccc(-n3cc(NC(=O)Nc4ccccc4Cl)cn3)c2)c(C)n1 ; O=C1Cc2cc(NC(=O)c3cccc(-n4cc(NC(=O)Nc5ccccc5Cl)cn4)c3)ccc2N1 ; Cc1ccc(NC(=O)c2ccc(F)c(-n3cc(NC(=O)Nc4ccccc4Cl)cn3)c2)cn1 ; O=C(Nc1cnn(-c2cccc(C(=O)Nc3ccnc4cccnc34)c2)c1)Nc1ccccc1Cl ; Cc1cc(NC(=O)c2cccc(-n3cc(NC(=O)N4Cc5ccccc5C4)cn3)c2)ccn1] 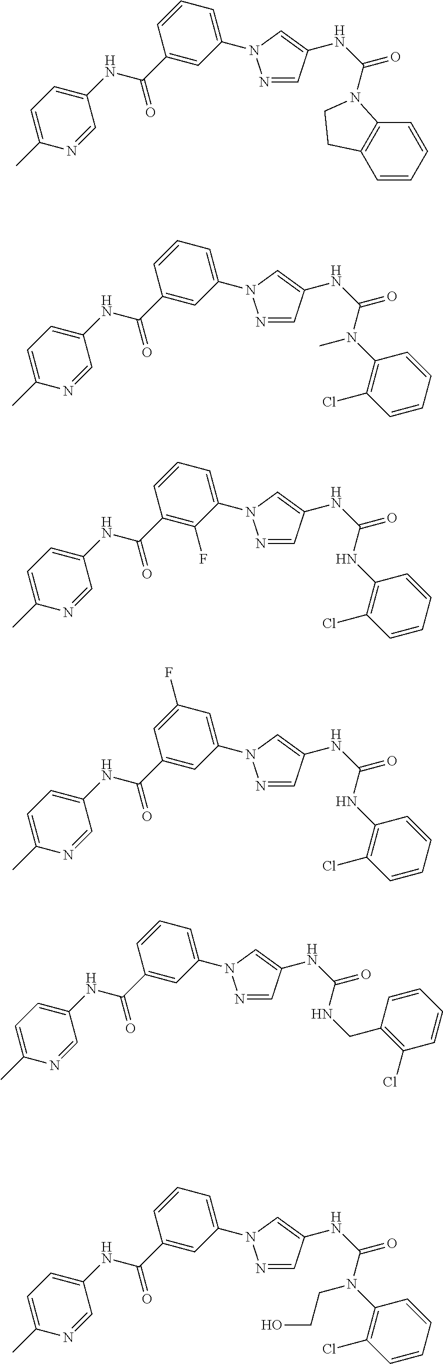 [SMILES: Cc1ccc(NC(=O)c2cccc(-n3cc(NC(=O)N4CCc5ccccc54)cn3)c2)cn1 ; Cc1ccc(NC(=O)c2cccc(-n3cc(NC(=O)N(C)c4ccccc4Cl)cn3)c2)cn1 ; Cc1ccc(NC(=O)c2cccc(-n3cc(NC(=O)Nc4ccccc4Cl)cn3)c2F)cn1 ; Cc1ccc(NC(=O)c2cc(F)cc(-n3cc(NC(=O)Nc4ccccc4Cl)cn3)c2)cn1 ; Cc1ccc(NC(=O)c2cccc(-n3cc(NC(=O)NCc4ccccc4Cl)cn3)c2)cn1 ; Cc1ccc(NC(=O)c2cccc(-n3cc(NC(=O)N(CCO)c4ccccc4Cl)cn3)c2)cn1] 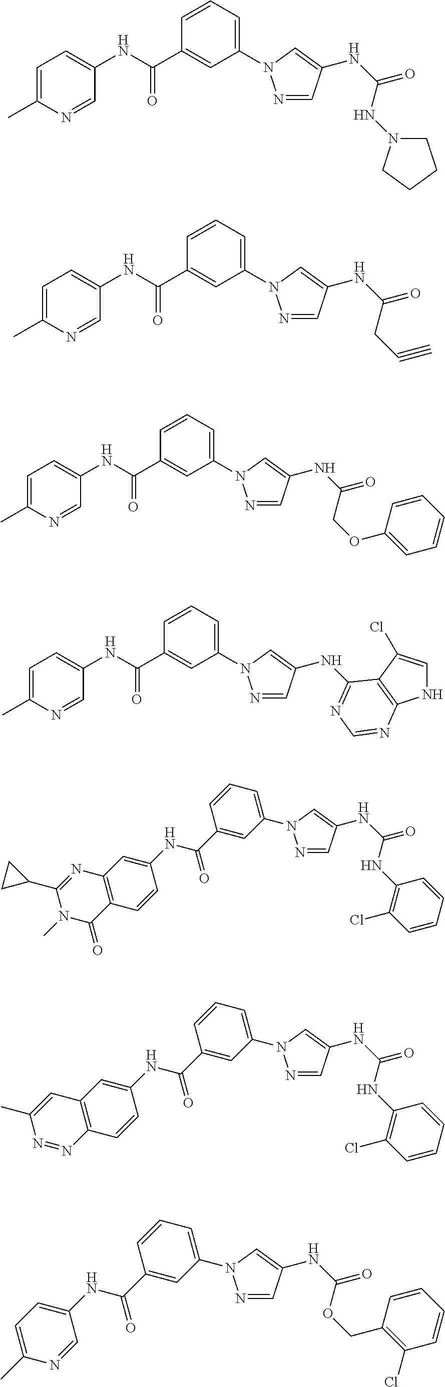 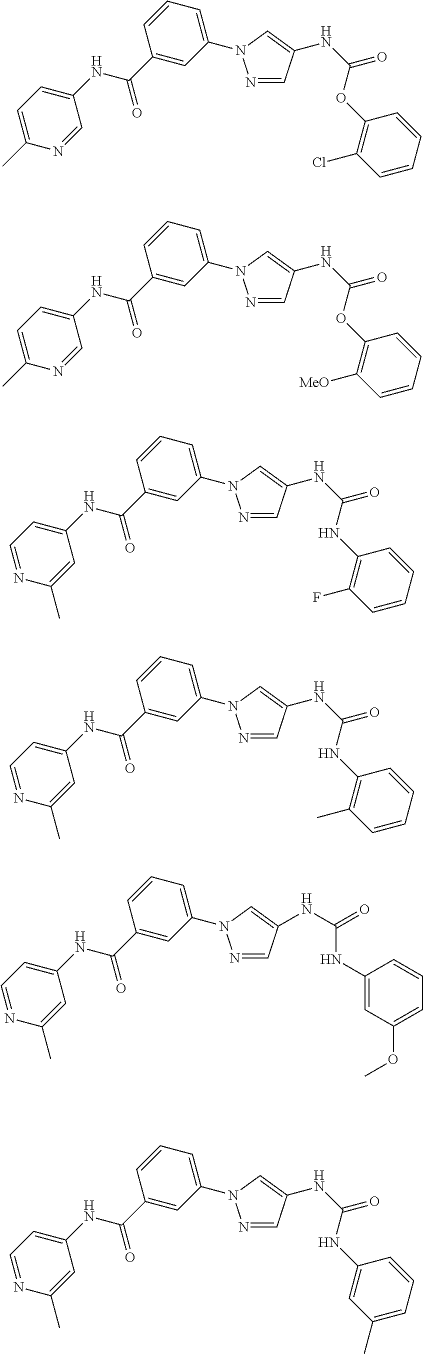 [SMILES: Cc1ccc(NC(=O)c2cccc(-n3cc(NC(=O)Oc4ccccc4Cl)cn3)c2)cn1 ; COc1ccccc1OC(=O)Nc1cnn(-c2cccc(C(=O)Nc3ccc(C)nc3)c2)c1 ; Cc1cc(NC(=O)c2cccc(-n3cc(NC(=O)Nc4ccccc4F)cn3)c2)ccn1 ; Cc1cc(NC(=O)c2cccc(-n3cc(NC(=O)Nc4ccccc4C)cn3)c2)ccn1 ; COc1cccc(NC(=O)Nc2cnn(-c3cccc(C(=O)Nc4ccnc(C)c4)c3)c2)c1 ; Cc1cccc(NC(=O)Nc2cnn(-c3cccc(C(=O)Nc4ccnc(C)c4)c3)c2)c1] 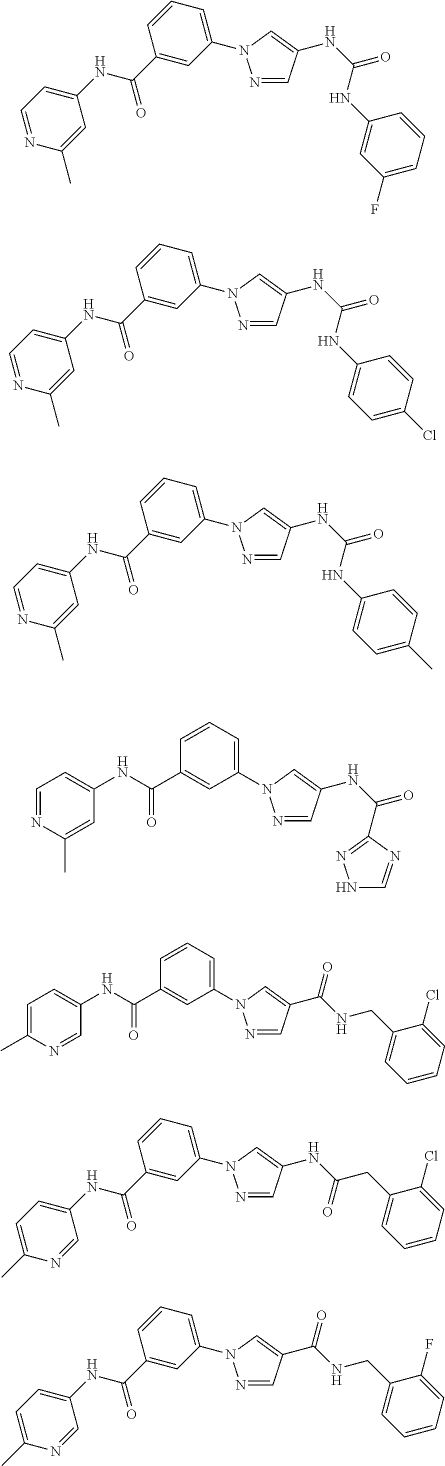 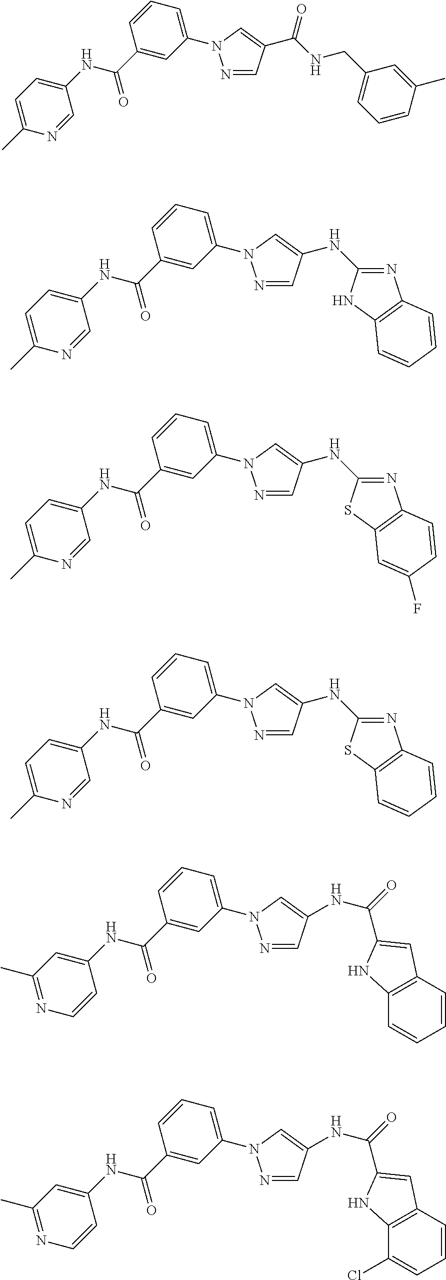 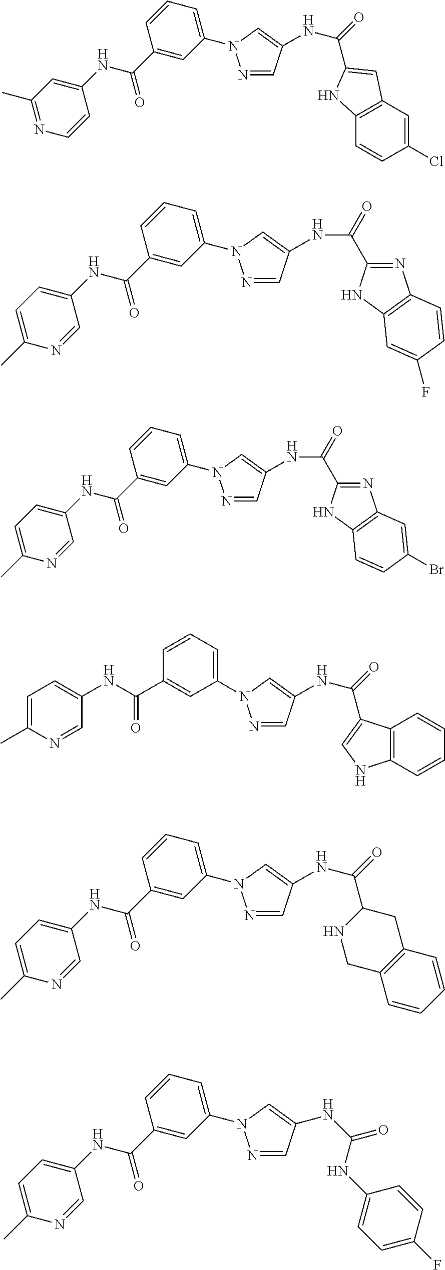 [SMILES: Cc1cc(NC(=O)c2cccc(-n3cc(NC(=O)c4cc5cc(Cl)ccc5[nH]4)cn3)c2)ccn1 ; Cc1ccc(NC(=O)c2cccc(-n3cc(NC(=O)c4nc5ccc(F)cc5[nH]4)cn3)c2)cn1 ; Cc1ccc(NC(=O)c2cccc(-n3cc(NC(=O)c4nc5cc(Br)ccc5[nH]4)cn3)c2)cn1 ; Cc1ccc(NC(=O)c2cccc(-n3cc(NC(=O)c4c[nH]c5ccccc45)cn3)c2)cn1 ; Cc1ccc(NC(=O)c2cccc(-n3cc(NC(=O)C4Cc5ccccc5CN4)cn3)c2)cn1 ; Cc1ccc(NC(=O)c2cccc(-n3cc(NC(=O)Nc4ccc(F)cc4)cn3)c2)cn1] 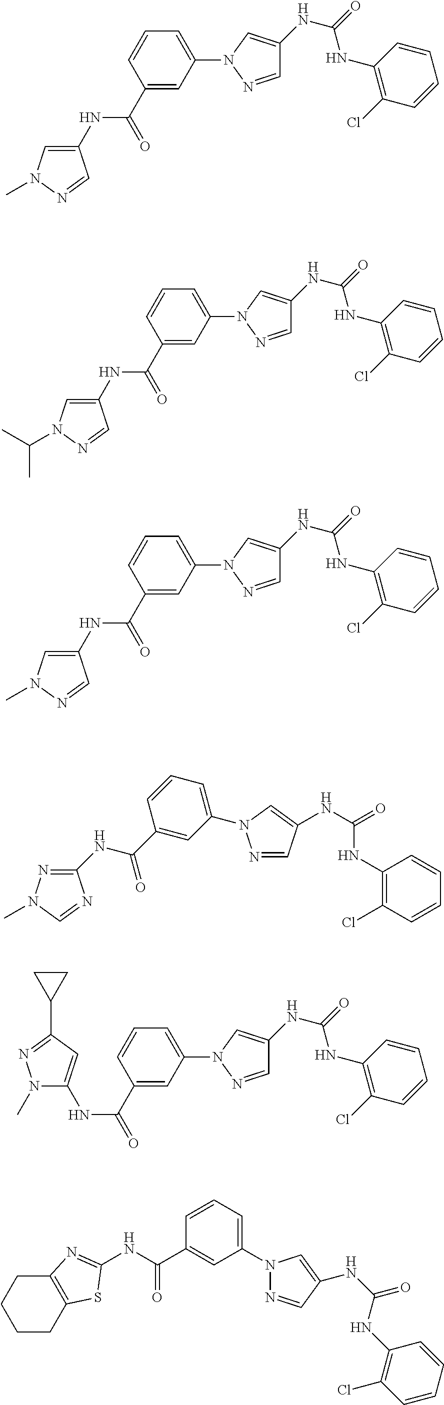 [SMILES: Cn1cc(NC(=O)c2cccc(-n3cc(NC(=O)Nc4ccccc4Cl)cn3)c2)cn1 ; CC(C)n1cc(NC(=O)c2cccc(-n3cc(NC(=O)Nc4ccccc4Cl)cn3)c2)cn1 ; Cn1cc(NC(=O)c2cccc(-n3cc(NC(=O)Nc4ccccc4Cl)cn3)c2)cn1 ; Cn1cnc(NC(=O)c2cccc(-n3cc(NC(=O)Nc4ccccc4Cl)cn3)c2)n1 ; Cn1nc(C2CC2)cc1NC(=O)c1cccc(-n2cc(NC(=O)Nc3ccccc3Cl)cn2)c1 ; O=C(Nc1cnn(-c2cccc(C(=O)Nc3nc4c(s3)CCCC4)c2)c1)Nc1ccccc1Cl] 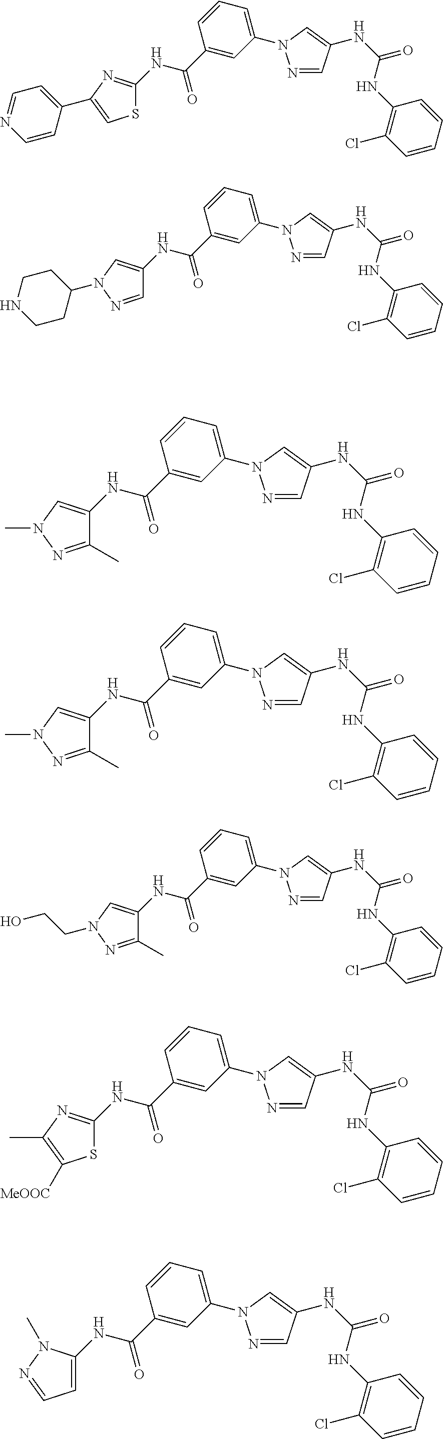 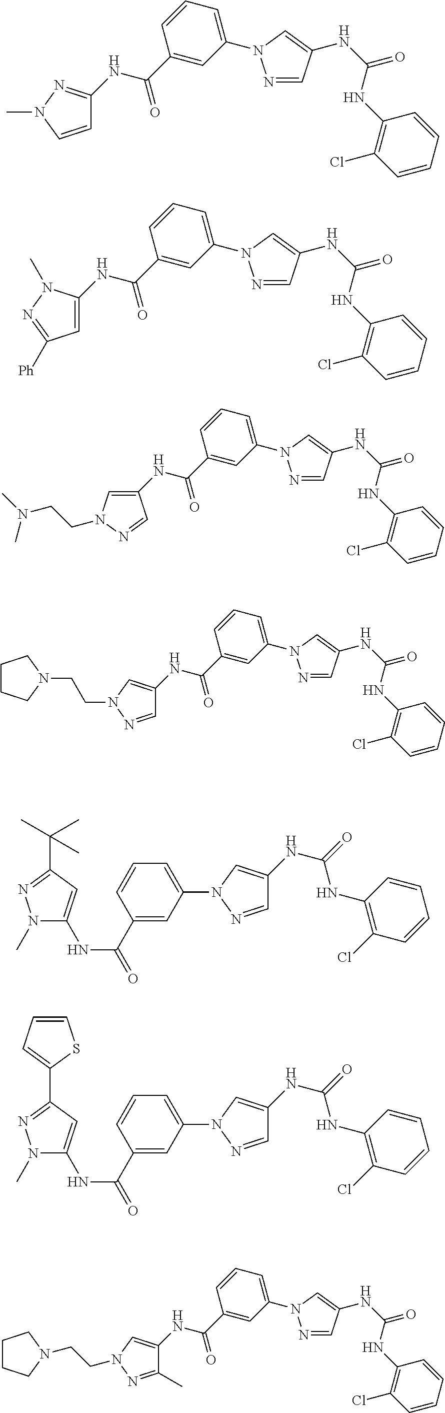 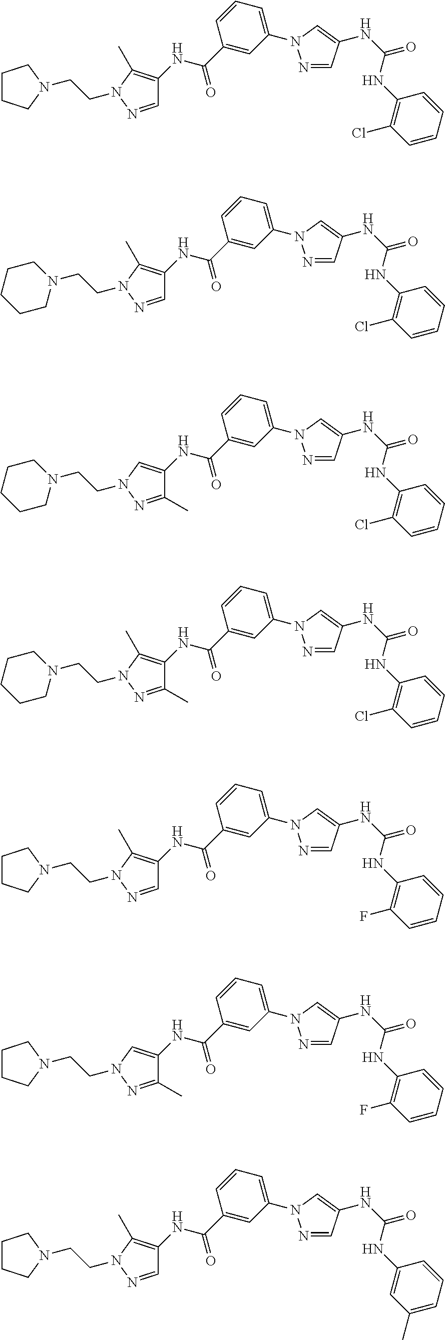 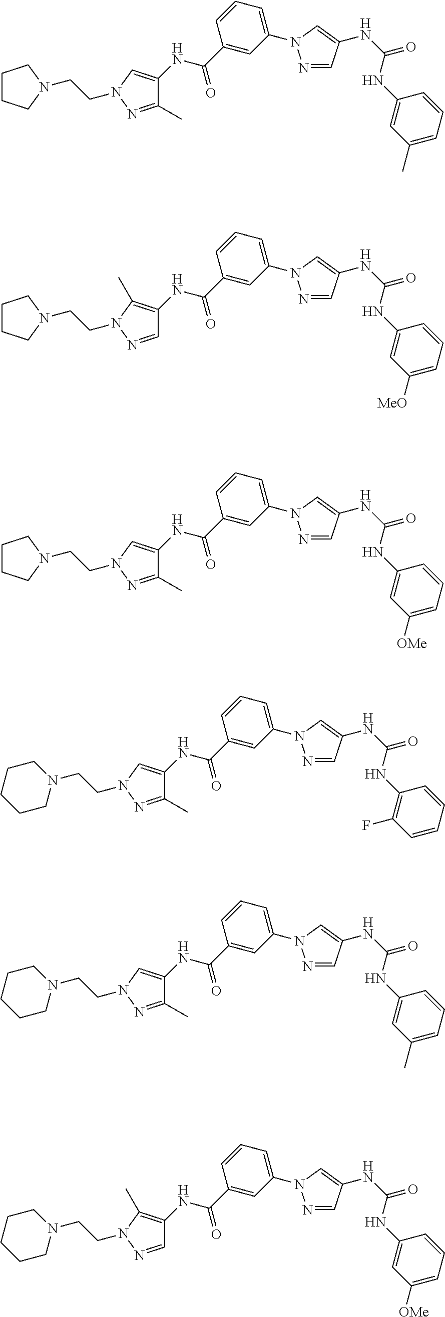 [SMILES: Cc1cccc(NC(=O)Nc2cnn(-c3cccc(C(=O)Nc4cn(CCN5CCCC5)nc4C)c3)c2)c1 ; COc1cccc(NC(=O)Nc2cnn(-c3cccc(C(=O)Nc4cnn(CCN5CCCC5)c4C)c3)c2)c1 ; COc1cccc(NC(=O)Nc2cnn(-c3cccc(C(=O)Nc4cn(CCN5CCCC5)nc4C)c3)c2)c1 ; Cc1nn(CCN2CCCCC2)cc1NC(=O)c1cccc(-n2cc(NC(=O)Nc3ccccc3F)cn2)c1 ; Cc1cccc(NC(=O)Nc2cnn(-c3cccc(C(=O)Nc4cn(CCN5CCCCC5)nc4C)c3)c2)c1 ; COc1cccc(NC(=O)Nc2cnn(-c3cccc(C(=O)Nc4cnn(CCN5CCCCC5)c4C)c3)c2)c1] 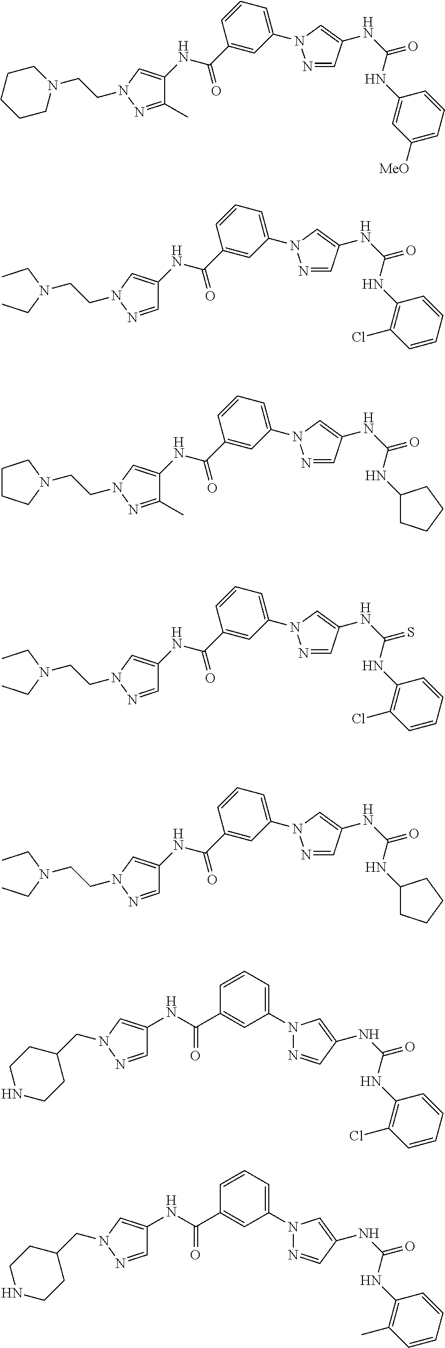 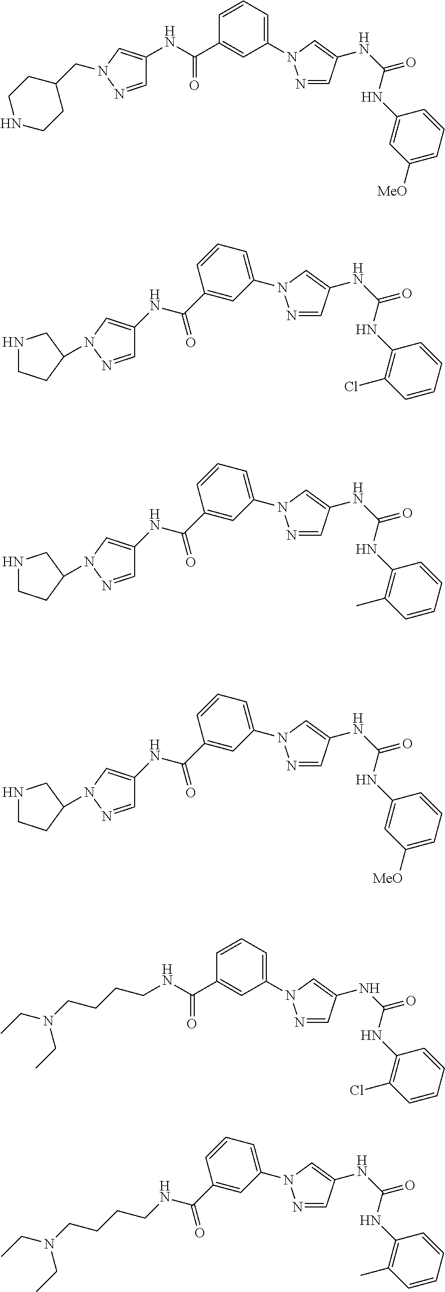 [SMILES: COc1cccc(NC(=O)Nc2cnn(-c3cccc(C(=O)Nc4cnn(CC5CCNCC5)c4)c3)c2)c1 ; O=C(Nc1cnn(-c2cccc(C(=O)Nc3cnn(C4CCNC4)c3)c2)c1)Nc1ccccc1Cl ; Cc1ccccc1NC(=O)Nc1cnn(-c2cccc(C(=O)Nc3cnn(C4CCNC4)c3)c2)c1 ; COc1cccc(NC(=O)Nc2cnn(-c3cccc(C(=O)Nc4cnn(C5CCNC5)c4)c3)c2)c1 ; CCN(CC)CCCCNC(=O)c1cccc(-n2cc(NC(=O)Nc3ccccc3Cl)cn2)c1 ; CCN(CC)CCCCNC(=O)c1cccc(-n2cc(NC(=O)Nc3ccccc3C)cn2)c1] 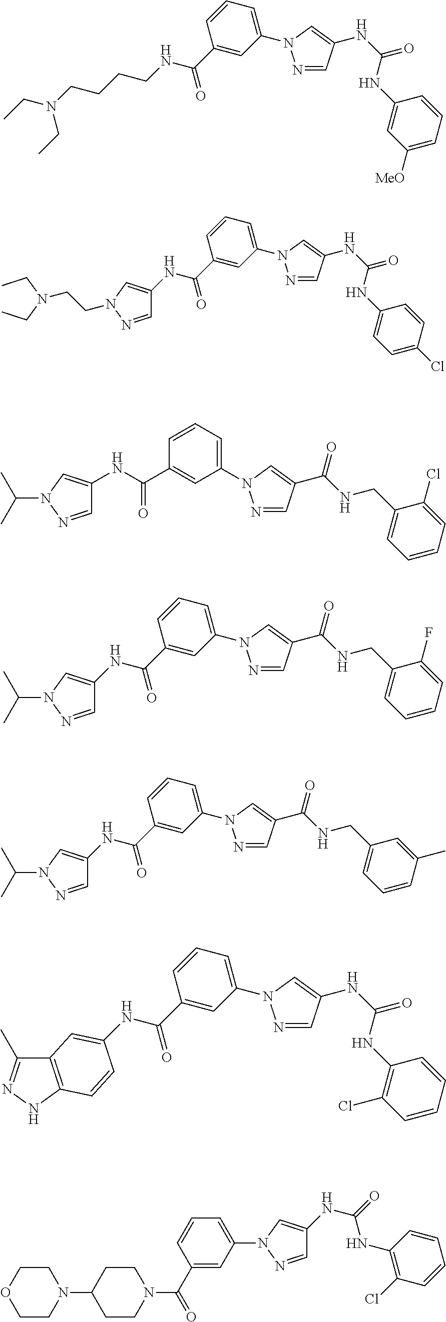 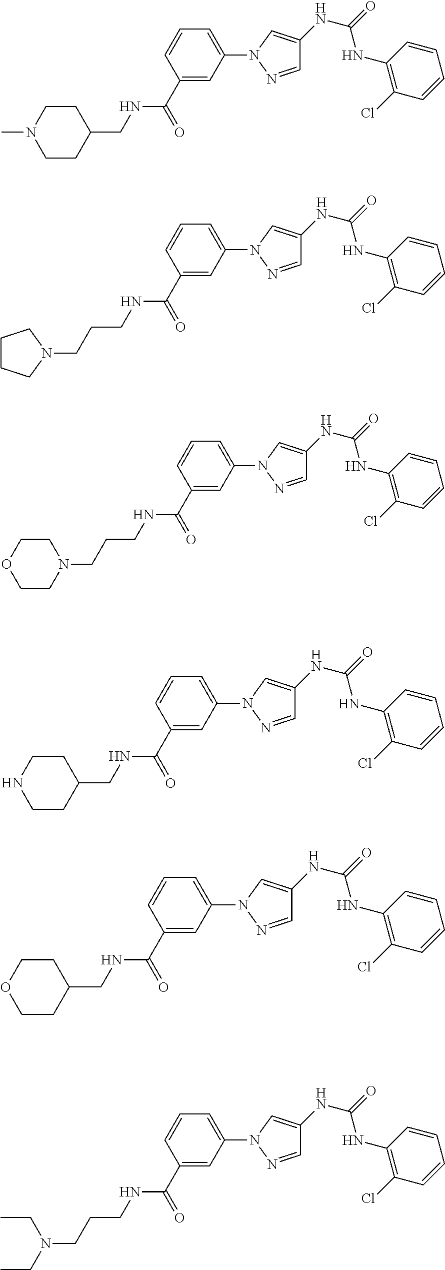 [SMILES: CN1CCC(CNC(=O)c2cccc(-n3cc(NC(=O)Nc4ccccc4Cl)cn3)c2)CC1 ; O=C(Nc1cnn(-c2cccc(C(=O)NCCCN3CCCC3)c2)c1)Nc1ccccc1Cl ; O=C(Nc1cnn(-c2cccc(C(=O)NCCCN3CCOCC3)c2)c1)Nc1ccccc1Cl ; O=C(Nc1cnn(-c2cccc(C(=O)NCC3CCNCC3)c2)c1)Nc1ccccc1Cl ; O=C(Nc1cnn(-c2cccc(C(=O)NCC3CCOCC3)c2)c1)Nc1ccccc1Cl ; CCN(CC)CCCNC(=O)c1cccc(-n2cc(NC(=O)Nc3ccccc3Cl)cn2)c1] 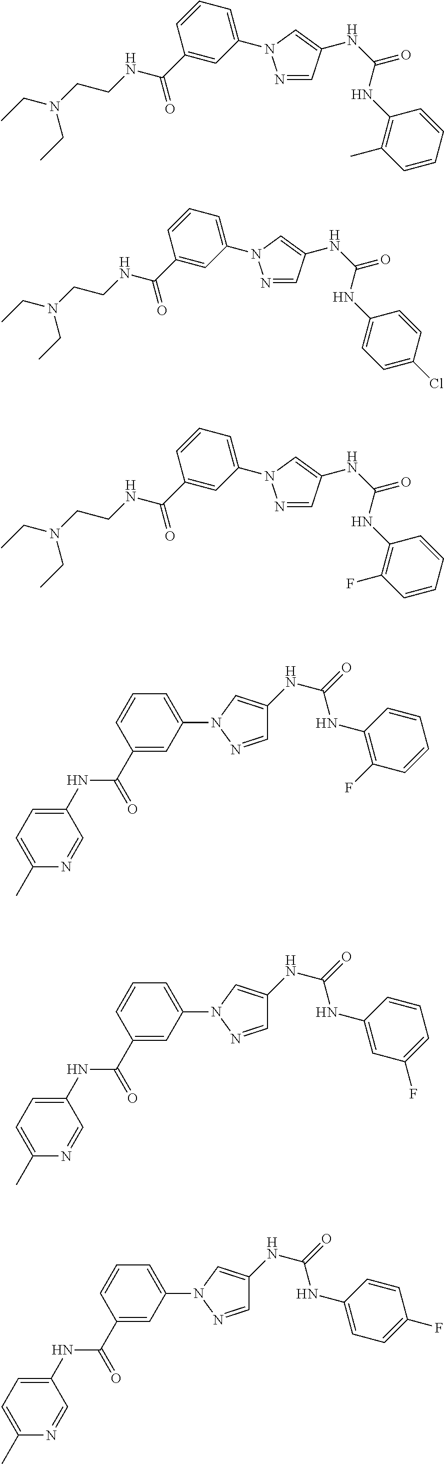 [SMILES: CCN(CC)CCNC(=O)c1cccc(-n2cc(NC(=O)Nc3ccccc3C)cn2)c1 ; CCN(CC)CCNC(=O)c1cccc(-n2cc(NC(=O)Nc3ccc(Cl)cc3)cn2)c1 ; CCN(CC)CCNC(=O)c1cccc(-n2cc(NC(=O)Nc3ccccc3F)cn2)c1 ; Cc1ccc(NC(=O)c2cccc(-n3cc(NC(=O)Nc4ccccc4F)cn3)c2)cn1 ; Cc1ccc(NC(=O)c2cccc(-n3cc(NC(=O)Nc4cccc(F)c4)cn3)c2)cn1 ; Cc1ccc(NC(=O)c2cccc(-n3cc(NC(=O)Nc4ccc(F)cc4)cn3)c2)cn1] 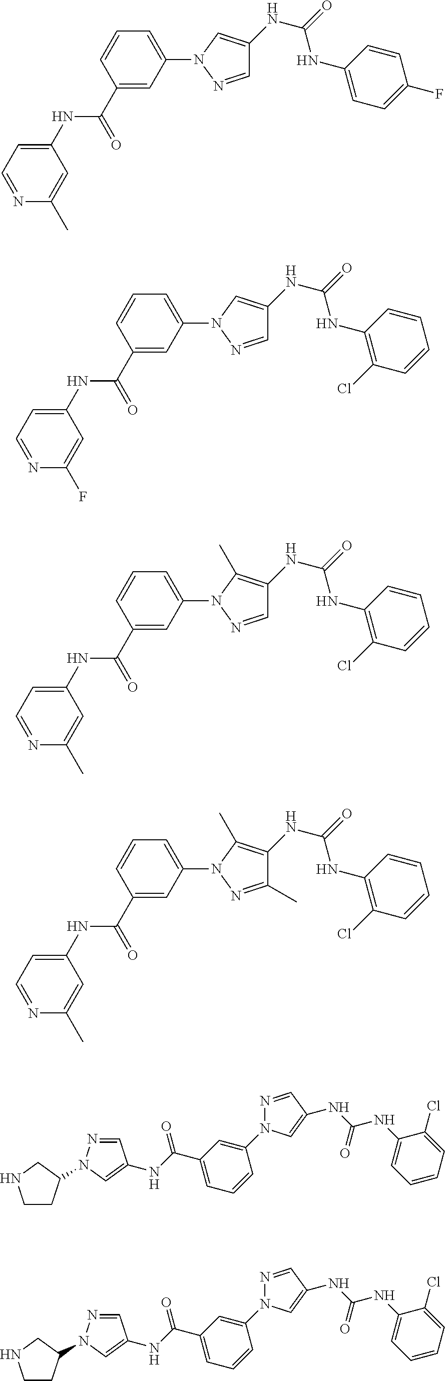 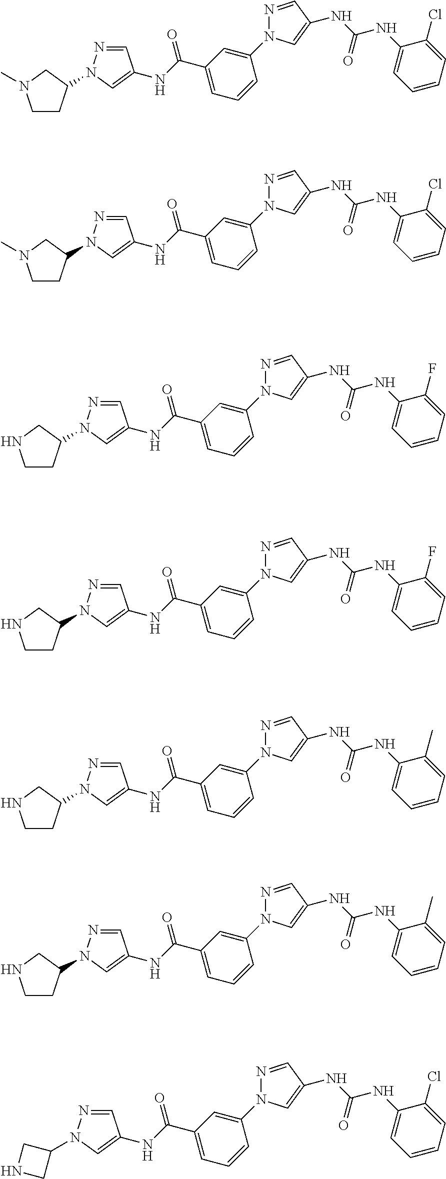 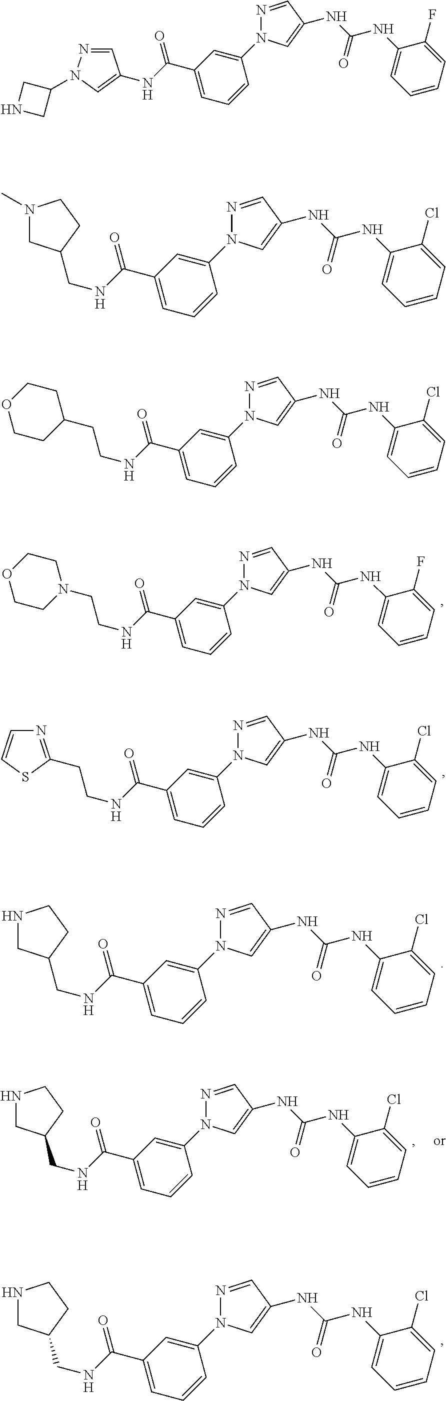
or a salt thereof.

11. A compound of formula (II) 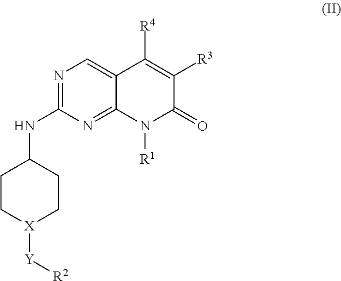
wherein
X is N or CH; when X is N, Y is absent; when X is CH, Y is NR′ or is O;
R¹ is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} (wherein q is 0, 1, or 2), O, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;
R² is H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} (wherein q is 0, 1, or 2), O, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;
or R² is (C₆-C₁₀) aryl, (C₆-C₁₀)aryl(C₁-C₆)alkyl, a 5-10 membered heteroaryl, or a 5-10 membered heteroaryl-(C₁-C₆)alkyl, wherein any aryl or heteroaryl is unsubstituted or is substituted with 1, 2, or 3 J groups; or R² is C(═O)OR, C(═O)R, or C(═O)NR₂;
R and R′ are independently at each occurrence H or (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} (wherein q is 0, 1, or 2), O, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;
R³ and R⁴ are each independently H, CF₃, (C₁-C₈)alkyl, (C₃-C₉)cycloalkyl, or (C₃-C₉)cycloalkyl(C₁-C₈)alkyl, wherein 0, 1, or 2 carbon atoms of the alkyl or cycloalkyl are replaced by a group independently selected from the set consisting of NR′, S(O)_{q} (wherein q is 0, 1, or 2), O, C(═S), C(═O), C(═O)O, C(═O)C(═O), C(═O)NR′, NR′C(═O), NR′C(═O)O, OC(═O)NR′, SO₂NR′, NR'SO₂, NR′SO₂NR′, C(═O)NR′NR′, or NR′C(═O)NR′;
or a pharmaceutically acceptable salt thereof.

12. The compound of claim 11, wherein X is N and Y is absent.

13. The compound of claim 11, wherein X is CH and Y is NR′.

14. The compound of claim 11, wherein X is CH and Y is O.

15. The compound of claim 11, wherein R³ and R⁴ are each H.

16. The compound of claim 11, wherein the compound is any one of 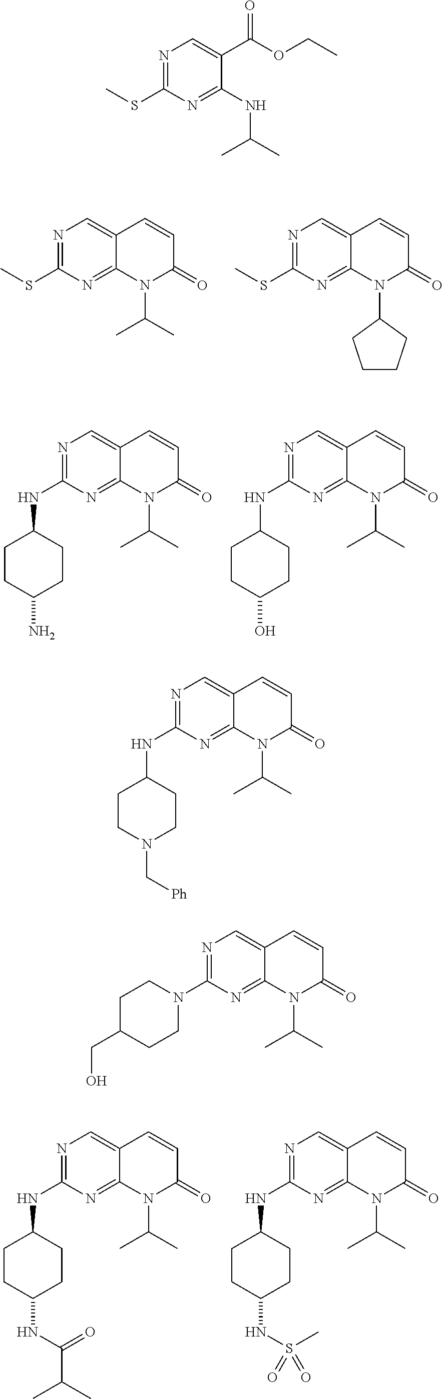 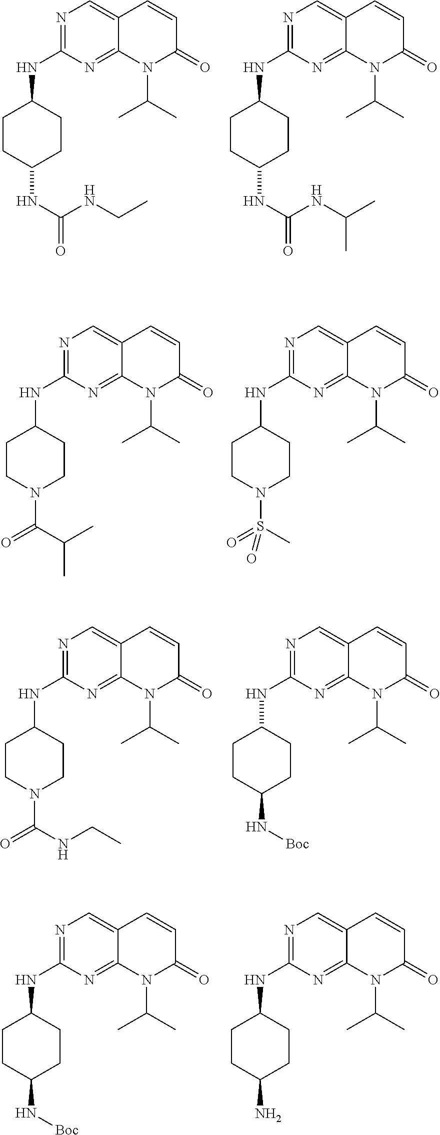 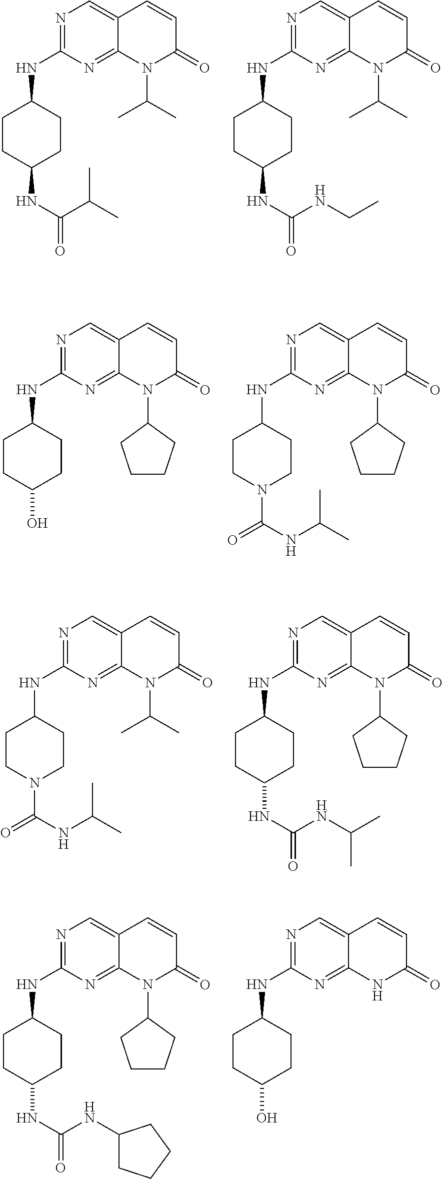 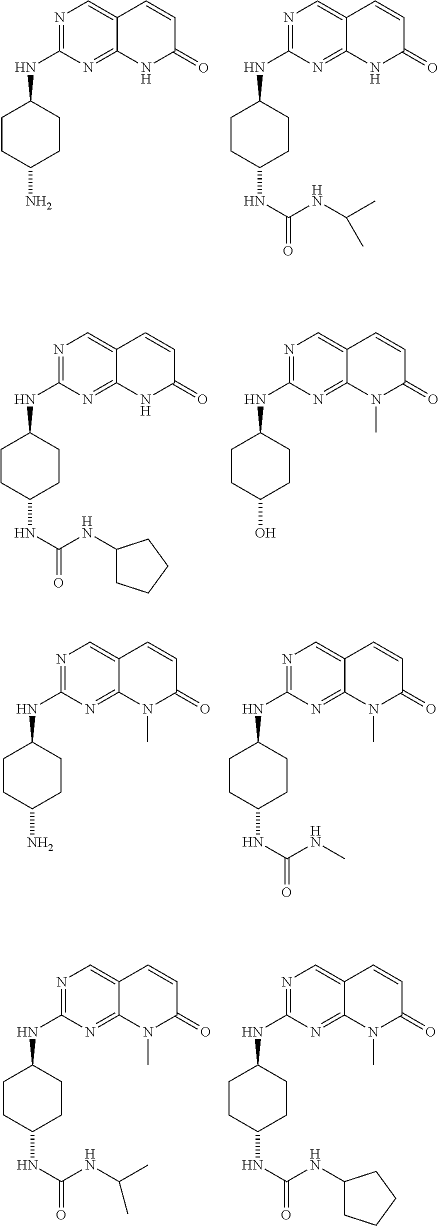 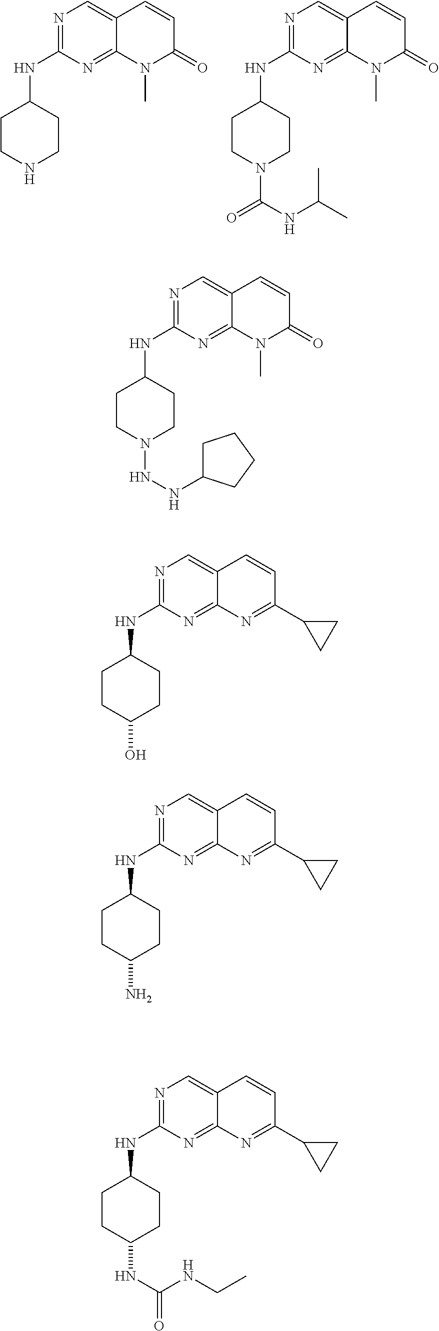 [SMILES: Cn1c(=O)ccc2cnc(NC3CCNCC3)nc21 ; CC(C)NC(=O)N1CCC(Nc2ncc3ccc(=O)n(C)c3n2)CC1 ; Cn1c(=O)ccc2cnc(NC3CCN(NNC4CCCC4)CC3)nc21 ; O[C@H]1CC[C@H](Nc2ncc3ccc(C4CC4)nc3n2)CC1 ; N[C@H]1CC[C@H](Nc2ncc3ccc(C4CC4)nc3n2)CC1 ; CCNC(=O)N[C@H]1CC[C@H](Nc2ncc3ccc(C4CC4)nc3n2)CC1] 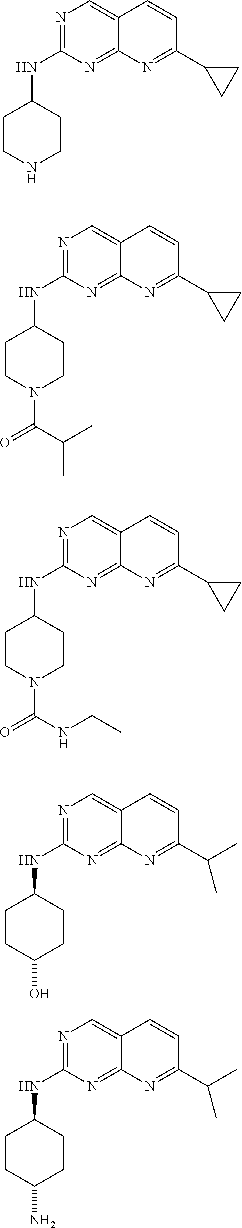 [SMILES: c1cc2cnc(NC3CCNCC3)nc2nc1C1CC1 ; CC(C)C(=O)N1CCC(Nc2ncc3ccc(C4CC4)nc3n2)CC1 ; CCNC(=O)N1CCC(Nc2ncc3ccc(C4CC4)nc3n2)CC1 ; CC(C)c1ccc2cnc(N[C@H]3CC[C@H](O)CC3)nc2n1 ; CC(C)c1ccc2cnc(N[C@H]3CC[C@H](N)CC3)nc2n1] 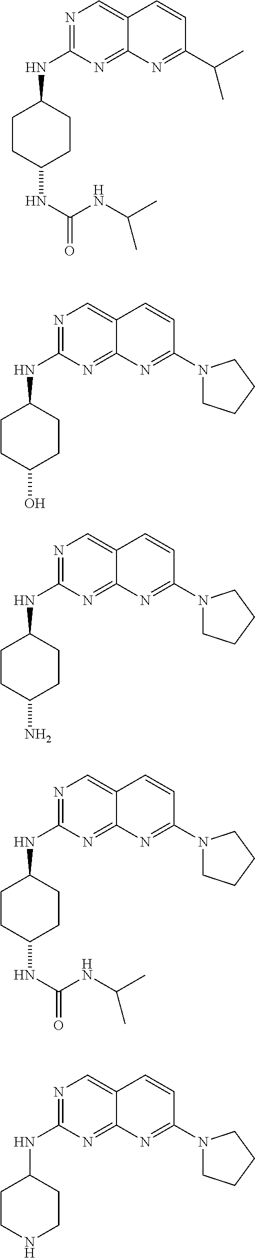 [SMILES: CC(C)NC(=O)N[C@H]1CC[C@H](Nc2ncc3ccc(C(C)C)nc3n2)CC1 ; O[C@H]1CC[C@H](Nc2ncc3ccc(N4CCCC4)nc3n2)CC1 ; N[C@H]1CC[C@H](Nc2ncc3ccc(N4CCCC4)nc3n2)CC1 ; CC(C)NC(=O)N[C@H]1CC[C@H](Nc2ncc3ccc(N4CCCC4)nc3n2)CC1 ; c1cc2cnc(NC3CCNCC3)nc2nc1N1CCCC1] 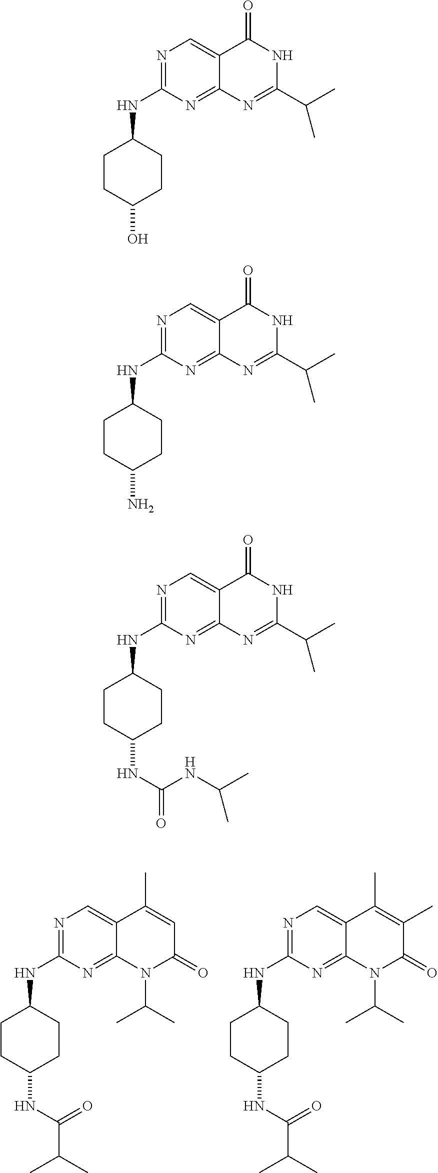 [SMILES: CC(C)c1nc2nc(N[C@H]3CC[C@H](O)CC3)ncc2c(=O)[nH]1 ; CC(C)c1nc2nc(N[C@H]3CC[C@H](N)CC3)ncc2c(=O)[nH]1 ; CC(C)NC(=O)N[C@H]1CC[C@H](Nc2ncc3c(=O)[nH]c(C(C)C)nc3n2)CC1 ; Cc1cc(=O)n(C(C)C)c2nc(N[C@H]3CC[C@H](NC(=O)C(C)C)CC3)ncc12 ; Cc1c(C)c2cnc(N[C@H]3CC[C@H](NC(=O)C(C)C)CC3)nc2n(C(C)C)c1=O] 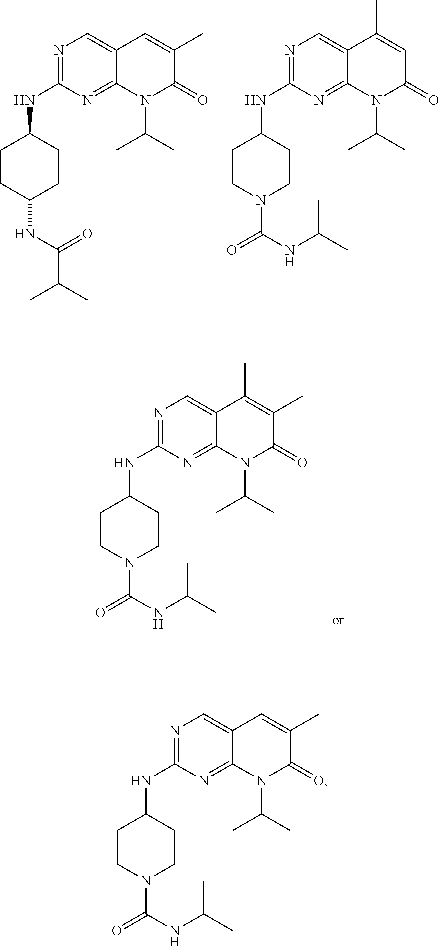 [SMILES: Cc1cc2cnc(N[C@H]3CC[C@H](NC(=O)C(C)C)CC3)nc2n(C(C)C)c1=O ; Cc1cc(=O)n(C(C)C)c2nc(NC3CCN(C(=O)NC(C)C)CC3)ncc12 ; Cc1c(C)c2cnc(NC3CCN(C(=O)NC(C)C)CC3)nc2n(C(C)C)c1=O ; Cc1cc2cnc(NC3CCN(C(=O)NC(C)C)CC3)nc2n(C(C)C)c1=O]
or a pharmaceutically acceptable salt thereof.

17. A pharmaceutical composition comprising the compound of claim 1 and a pharmaceutically acceptable excipient.

18.-20. (canceled)

21. A method of treatment of a disorder in a patient wherein inhibition of a kinase is medically indicated, comprising administration of an effective dose of a compound of claim 1.

22. The method of claim 21 wherein the kinase is JNK isoform 2 or isoform 3.

23. The method of claim 21 wherein the disorder is Parkinson's disease (PD) Alzheimer's disease (AD), Huntington's disease (HD), amyotrophic lateral sclerosis (ALS), multiple sclerosis (MS), myocardial infarction (MI), glaucoma, obesity, diabetes, cancer, rheumatoid arthritis, fibrotic disease, pulmonary fibrosis, kidney disease, liver inflammation, Crohn's disease, hearing loss, Prader Will syndrome, or a condition where modification of feeding behavior is medically indicated.
